(19) United States
(12) Patent Application Publication
(10) Pub. No.: US 2026/0327234 A1
(43) Pub. Date: Oct. 22, 2026

(54) NOVEL COMPOUNDS AS CYP11A1 INHIBITORS AND METHODS OF USE THEREOF

(71) Applicants: Shenzhen Ionova Life Science Co., Ltd., Shenzhen (CN); Foshan lonova Biotherapeutics Co., Inc., Foshan (CN); Guangdong Touchstone Translational Research Institute Co., Ltd., Foshan (CN)

(72) Inventors: Zilei Xia, Shenzhen (CN); Jing Su, Scotch Plains, NJ (US); Yinfa Yan, Shenzhen (CN); Chang Bai, Shenzhen (CN); Lishan Lin, Shenzhen (CN); Yongkui Sun, Shenzhen (CN)

(21) Appl. No.: 19/483,040

(22) PCT Filed: May 11, 2024
(86) PCT No.: PCT/CN2024/092564

§ 371 Date: Nov. 10, 2025

Related U.S. Application Data
(60) Provisional application No. 63/504,611, filed on May 26, 2023.
(60) Provisional application No. 63/594,170, filed on Oct. 30, 2023.
(60) Provisional application No. 63/616,921, filed on Jan. 2, 2024.

(30) Foreign Application Priority Data
May 11, 2023 (WO) PCT/CN2023/093491
Sep. 20, 2023 (WO) PCT/CN2023/119980
Jan. 4, 2024 (WO) PCT/CN2024/070542

(51) Int. Cl. C07D 487/04; A61K 31/4035; A61K 31/423; A61K 31/438; A61K 31/4439; A61K 31/454; A61K 31/4545; A61K 31/4709; A61K 31/4725; A61K 31/498; A61K 31/4985; A61K 31/506; A61K 31/517; A61K 31/519; C07D 401/10; C07D 401/12; C07D 401/14; C07D 405/06; C07D 405/14; C07D 409/14; C07D 413/06; C07D 417/14; C07D 471/04; C07D 491/052; C07D 493/04; C07D 495/04; C07D 519/00

(52) U.S. Cl. CPC C07D 487/04; A61K 31/4035; A61K 31/423; A61K 31/438; A61K 31/4439; A61K 31/454; A61K 31/4545; A61K 31/4709; A61K 31/4725; A61K 31/498; A61K 31/4985; A61K 31/506; A61K 31/517; A61K 31/519; C07D 401/10; C07D 401/12; C07D 401/14; C07D 405/06; C07D 405/14; C07D 409/14; C07D 413/06; C07D 417/14; C07D 471/04; C07D 491/052; C07D 493/04; C07D 495/04; C07D 519/00

(57) ABSTRACT

The present invention provides novel compounds of Formula (I) as CYP11A1 inhibitors, which are useful in the treatment of conditions and diseases mediated by steroid hormone receptor, e.g., androgen receptor (AR) dependent conditions and diseases, including prostate cancer. Preparation methods, pharmaceutical compositions and methods of use are also included.

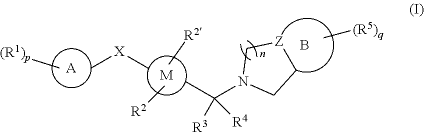

### CROSS-REFERENCE TO RELATED APPLICATIONS

The present application claims the benefit of PCT Application No. PCT/CN2023/093491, filed on May 11, 2023; U.S. Application No. 63/504,611, filed on May 26, 2023; PCT Application No. PCT/CN2023/119980, filed on Sep. 20, 2023; U.S. Application No. 63/594,170, filed on Oct. 30, 2023; U.S. Application No. 63/616,921, filed on Jan. 2, 2024; and PCT Application No. PCT/CN2024/070542, filed on Jan. 4, 2024; the contents of all of which are hereby incorporated by reference in their entirety.

### BACKGROUND OF THE INVENTION

Prostate cancer (PC) is one of the most frequently diagnosed cancers in men and a leading cause of male cancer-related mortality worldwide. Advanced stage prostate cancer has a poor prognosis which is reflected by a five-year survival rate of 31-36%. It is widely accepted that androgen and androgen receptor (AR) play an important role in the occurrence and progression of prostate cancer. Therefore, androgen deprivation therapy (ADT) such as surgical or chemical castration is the principal therapeutic strategy for treatment of castration-sensitive prostate cancer (CSPC). However, despite an initial favorable response to ADT, almost all patients inevitably progress from CSPC to a more aggressive phenotype known as Castration-resistant prostate cancer (CRPC) (Transl Androl Urol. 2021 October; 10(10): 3918-3930).

Multiple mechanisms contribute to the progression of castration resistant disease. Evidence now supports the concept that the progression from a hormone-naïve state to a castration-resistant phenotype is related to continued transactivation of androgen receptor (AR), and AR remains an important driver in this progression. Detailed studies on CRPC have shown its dependency on the AR signaling axis despite systemic depletion of androgens by various mechanisms (J Carcinog. 2011; 10: 20; Transl Androl Urol. 2015 June; 4(3): 365-380). These findings promote development of new agents to inhibit AR signaling, for examples, enzalutamide (Xtandi), which is an AR antagonist that prevents nuclear translocation and chromatin binding, and abiraterone acetate (Zytiga), which is an inhibitor of Cytochrome P450 17A1 (CYP17A1), leading to suppression of androgens derived from the adrenal glands, the prostate tumor and the tumor microenvironment. These new therapeutic agents provide additional survival benefit, however, acquired resistance to abiraterone or enzalutamide remains a clinical challenge (Oncogene, 2013 (32): 5501-5511).

Preclinical studies on CRPC have shown that extragonadal steroid hormones other than androgens may maintain cancer growth by activating the mutated or overexpressed AR. AR mutations in the ligand-binding domain (LBD) may change the conformation of AR and allow lower affinity steroid hormones to bind to the AR and promote cancer growth. And the lower affinity steroids may also be converted into higher affinity AR binding steroids within cancer tissues. Therefore, inhibition of the synthesis of most, or even all, steroid hormones might be beneficial for patients with steroid hormone dependent cancers, such as prostate cancer, even in advanced stages of the disease (Mol Cancer Ther, 2022, 21(12): 1765-1776).

Since all steroid hormones are produced from a single precursor cholesterol via a series of enzymatic reactions, and CYP11A1 is the only enzyme that catalyzes the first step of steroid hormone biosynthesis, inhibition of cytochrome P450 11A1 (CYP11A1) has been hypothesized to halt the synthesis of all steroid hormones. Hence, a compound having CYP11A1 inhibitory effect has the potential to inhibit all steroid hormone biosynthesis, and thus, prevent activation of a steroid hormone receptor, such as AR. As such, CYP11A1 inhibitors could be of interest in treating steroid hormone dependent cancers, such as CRPC (Mol Cancer Ther, 2022, 21(12): 1765-1776).

### BRIEF SUMMARY OF THE INVENTION

The present invention relates to a compound of Formula (I). Such compounds are potent CYP11A1 inhibitors, and useful in the treatment of steroid hormone dependent conditions and diseases. In particular, compounds according to the present invention are useful in the treatment of AR dependent conditions and diseases, including prostate cancer. Also provided are pharmaceutical compositions and methods of treating diseases mediated by steroid hormone receptor.

In one aspect, the invention provides a compound of Formula (I), or a pharmaceutically acceptable salt, tautomer, deuterated derivative, or stereoisomer thereof,

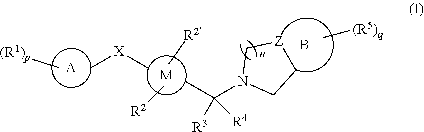

wherein
ring A is monocyclic, bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si), oxygen (O), sulfur (S) or nitrogen (N), and the ring-forming sulfur is optionally substituted with two oxo (═O) groups;
ring M is monocyclic or fused bicyclic ring, containing 0-4 ring-forming heteroatoms independently selected from oxygen (O), sulfur (S) or nitrogen (N); wherein a ring-forming carbon atom is optionally substituted with oxo (═O) to form a carbonyl group;
ring B is 5 or 6 membered aryl or heteroaryl, and is optionally substituted with one to three R⁵;
X is —CR⁶R⁷—O— or a bond;
n is 1 or 2;
Z is Cor N;
each R¹ independently is H, C₁₋₇ alkyl, C₂₋₇ alkenyl, C₂₋₇ alkynyl, amino, alkylamino, dialkylamino, alkoxy, alkoxyalkyl, hydroxyalkyl, halogen, haloalkyl, —CN, cyanoalkyl, —OR¹¹, —SO₂R¹¹, —SO₂NR¹²R^{12′}, —NR¹²SO₂R¹¹, —C(═O)—R¹¹, —COOR¹¹, —COONR¹²R^{12′}, —C(═O)—NR¹²R^{12′}, —P(═O)R¹²R^{12′}, cycloalkyl, heterocyclyl, aryl or heteroaryl, wherein cycloalkyl, heterocyclyl, aryl or heteroaryl is optionally substituted with one to three R¹⁰;
each of R² and R^{2′} is independently H, halogen, haloalkyl, alkoxy, alkoxyalkyl, hydroxyl, hydroxyalkyl, —CN, cyanoalkyl, C₁₋₇ alkyl, cycloalkyl or heterocyclyl, wherein cycloalkyl or heterocyclyl is optionally substituted with one or more R¹⁰;
each of R³ and R⁴ independently is H, alkyl, cycloalkyl or heterocyclyl, and is optionally substituted with one or more substituents independently selected from halogen, haloalkyl, —OH, hydroxyalkyl, alkoxy or —CN;
or, R³ and R⁴, together with the atom to which they are both bonded, optionally form a 3-7 membered monocyclic, bicyclic or spirocyclic cycloalkyl or heterocyclyl, wherein cycloalkyl or heterocyclyl is optionally substituted with one to three R¹⁰;
each R⁵ independently is H, alkyl, halogen, haloalkyl, —OH, hydroxyalkyl, alkoxy, haloalkoxy, alkoxyalkyl, —CN, cyanoalkyl, amino, alkylamino, dialkylamino, aryl, heteroaryl, cycloalkyl, or heterocyclyl, wherein the aryl, heteroaryl, cycloalkyl or heterocyclyl is independently and optionally substituted with one or more R¹⁰;
each of R⁶ and R⁷ independently is H, alkyl, cycloalkyl or heterocyclyl, and is optionally substituted with one or more R¹⁰;
or, R⁶ and R⁷, together with the atom to which they are both bonded, optionally form a 3-7 membered monocyclic, bicyclic or spirocyclic cycloalkyl or heterocyclyl; wherein bicyclic or spirocyclic cycloalkyl or heterocyclyl is optionally substituted with one to three R¹⁰;
each R¹⁰ independently is H, alkyl, halogen, haloalkyl, —OH, hydroxyalkyl, -alkoxy, -alkoxyalkyl, CN or cyanoalkyl;
each of R¹¹, R¹², and R^{12′} independently is H, alkyl, haloalkyl, aryl, heteroaryl, cycloalkyl, or heterocyclyl, each of which is optionally substituted with one or more R¹⁰;
or, R¹² and R^{12′}, together with the atom to which they are both bonded, optionally form a cycloalkyl or heterocyclyl, wherein cycloalkyl or heterocyclyl is optionally substituted with one to three R¹⁰;
the heterocyclyl in each occurrence with the provision not in ring A contains one to four ring-forming heteroatoms each of which is independently oxygen (O), sulfur (S) or nitrogen (N);
the heterocyclyl in each occurrence is a monocyclic, bicyclic, bridged bicyclic, fused bicyclic, spirocyclic or polycyclic ring; and
the heterocyclyl in each occurrence is unsaturated or partially saturated;
the heteroaryl in each occurrence contains one to four ring-forming heteroatoms each of which is independently oxygen (O), sulfur (S), or nitrogen (N);
any H is optionally replaced with deuterium (D);
p or q is independently 0, 1, 2, or 3;
   or a pharmaceutically acceptable salt, tautomer, or stereoisomer thereof.

In some embodiments, the compound is of Formula (II) or (III):

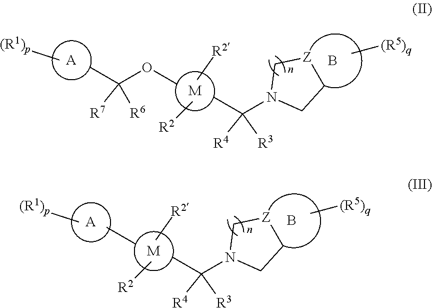

or a pharmaceutically acceptable salt, tautomer, or stereoisomer thereof. In Formula (II) or (III),
ring A is monocyclic, bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si), oxygen (O), sulfur (S) or nitrogen (N), and the ring-forming sulfur is optionally substituted with two oxo (═O) groups;
ring M is monocyclic or fused bicyclic ring, containing 0-4 ring-forming heteroatoms independently selected from N, O or S; wherein a ring-forming carbon atom is optionally substituted with oxo (═O) to form a carbonyl group;
ring B is 5 or 6 membered aryl or heteroaryl, and is optionally substituted with one to three R⁵.

In some embodiments, the compound is of Formula (IIa) or (IIIa):

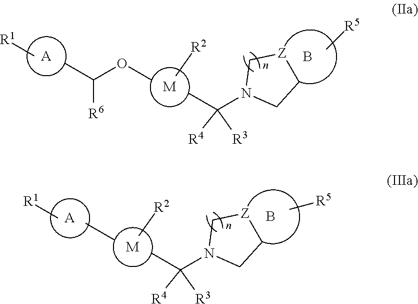

or a pharmaceutically acceptable salt, tautomer, or stereo isomer thereof. In Formula (IIa) or (IIIa),
ring A is monocyclic, fused bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si), oxygen (O), sulfur (5) or nitrogen (N), and the ring-forming sulfur is optionally substituted with two oxo (═O) groups;
ring M is monocyclic or fused bicyclic ring, containing 0-4 ring-forming heteroatoms independently selected from N, O or S; wherein a ring-forming carbon atom is optionally substituted with oxo (═O) to form a carbonyl group; and
ring B is 5 or 6 membered aryl or heteroaryl, and is optionally substituted with one to three R⁵.

In some embodiments,

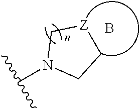

is

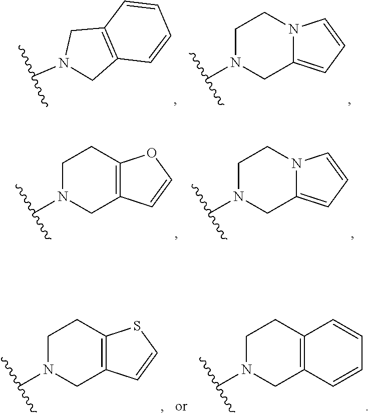 [SMILES: *N1Cc2ccccc2C1 ; *N1CCn2cccc2C1 ; *N1CCc2occc2C1 ; *N1CCn2cccc2C1 ; *N1CCc2sccc2C1 ; *N1CCc2ccccc2C1]

In some preferred embodiments,

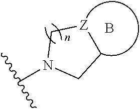

is

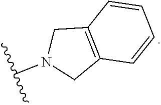 [SMILES: *N1Cc2ccccc2C1]

In some embodiments, the compound is of Formula (IV) or (V):

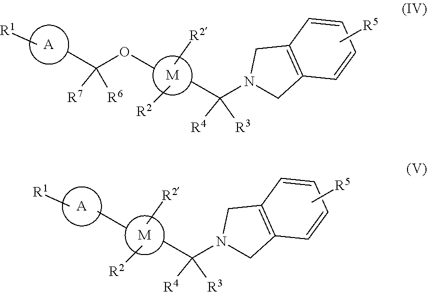

or a pharmaceutically acceptable salt, tautomer, or stereoisomer thereof. Formula (IV) or (V),
ring A is monocyclic, bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si) oxygen (O), sulfur (S) or nitrogen (N), and the ring-forming sulfur is optionally substituted with two oxo (═O) groups; and
ring M is monocyclic or fused bicyclic ring, containing 0-4 ring-forming heteroatoms independently selected from N, O or S; wherein a ring-forming carbon atom is optionally substituted with oxo (═O) to form a carbonyl group.

Examples of ring M include, but are not limited to, the following:

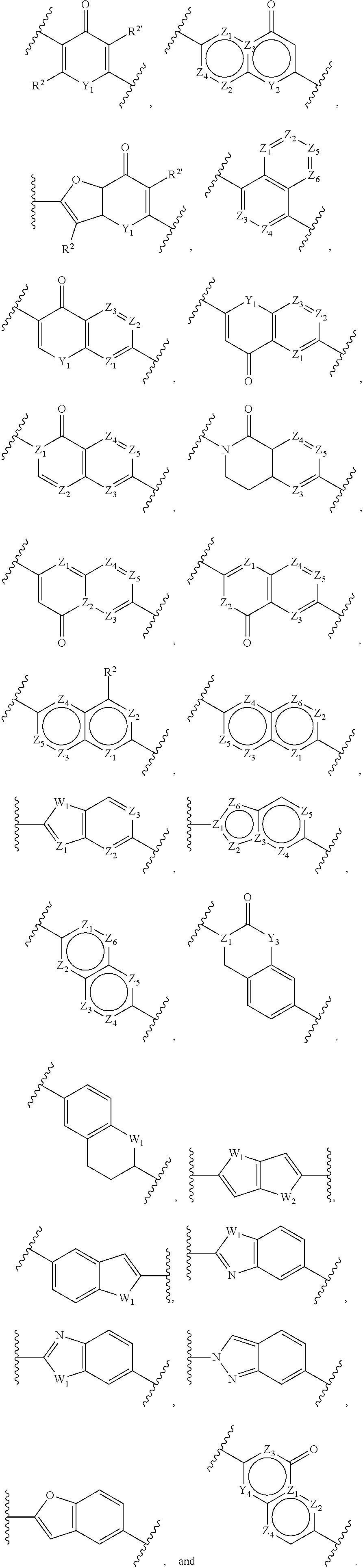

In each of these examples,
ring M is optionally substituted with R² and/or R^{2′};
Y₁ is NR, O or —CRR′—;
Y₂ is N, O or ═CR—;
Y₃ is O, NR, or —CRR′—;
Y₄ is N, O or CR;
each of W₁ and W₂ is independently O, S, NR, or —CRR′—;
each of Z₁, Z₂, Z₃, Z₄, Z₅ and Z₆ is independently N or CR; and
R or R′ is H, alkyl, or cycloalkyl; and
the dashed circle ( [symbol] ) within a ring indicates the ring contains 1, 2, or 3 double bonds as long as the valence allows.

In some embodiments, ring M is

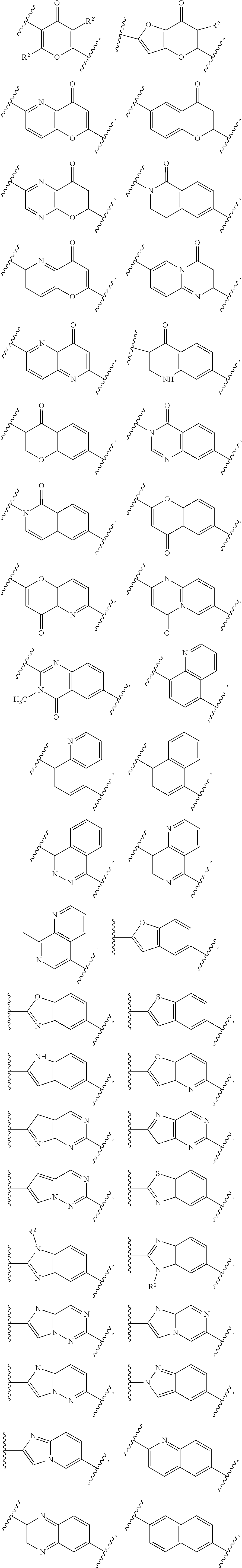

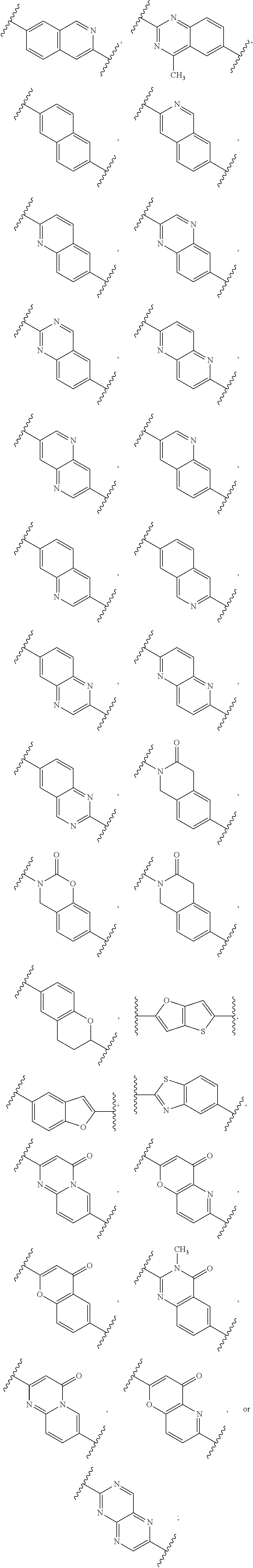

and ring M is optionally substituted with R² and/or R^{2′}.

Examples of ring A include, but are not limited to,

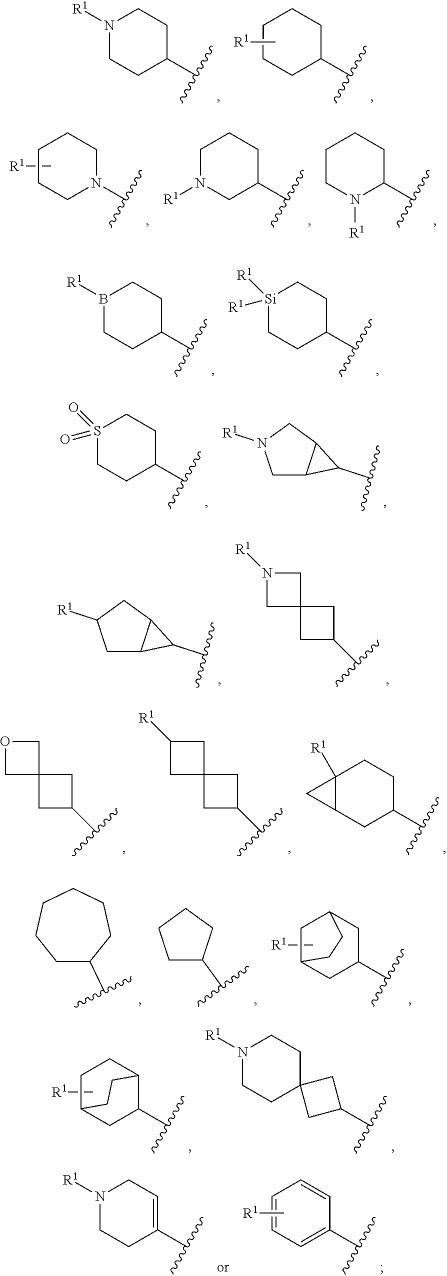

and ring A is optionally substituted with one to three R¹ Groups.

In some embodiments, R¹ is —OH, C₁₋₇alkyl, —CN, —SO₂R¹¹, —COOR¹¹, —C(═O)R¹¹, —NR¹²SO₂R¹¹, —C(═O)—NR¹²R^{12′}, —SO₂NR¹²R^{12′}, or —P(═O)R¹²R^{12′}, wherein each R¹¹ is independently H, cycloalkyl, heterocyclyl, haloalkyl, alkyl, aryl, or heteroaryl, wherein the aryl, heteroaryl, cycloalkyl and heterocyclyl are optionally substituted with one more alkyl, halo, or haloalkyl; R¹² and R^{12′} are each independently H, cycloalkyl, haloalkyl, or alkyl.

In some embodiments, R³ and R⁴ are each independently H or alkyl, and any H is optionally replaced by D; or, R³ and R⁴, together with the atom to which they are both bonded, form a 3- to 7-membered cycloalkyl.

In some embodiments, each of R⁶ and R⁷ independently is H or alkyl.

In some embodiments, halogen is —F or —Cl.

Exemplary compounds of the present invention, without any limitations, include:

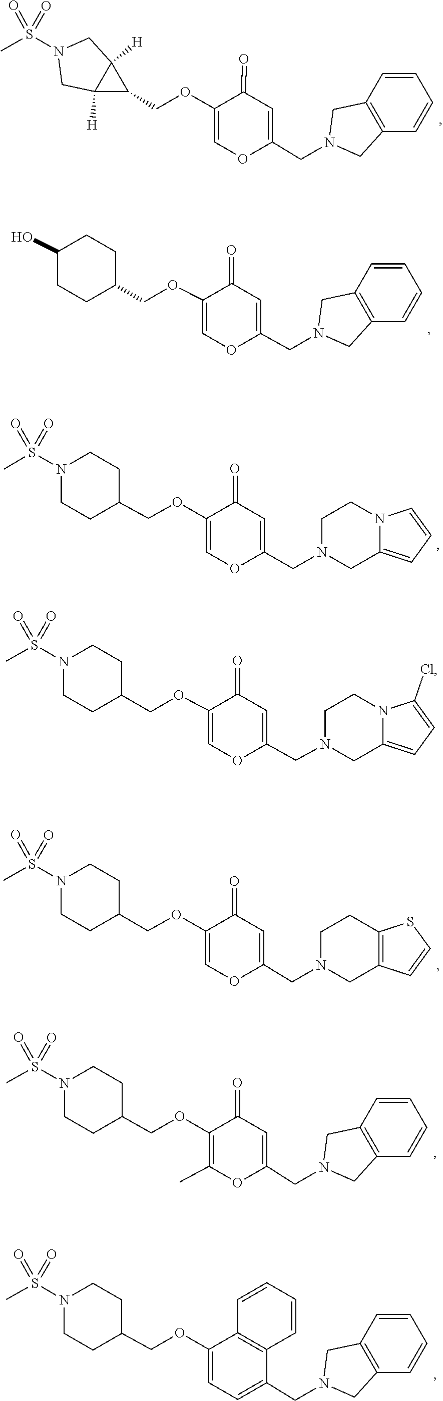

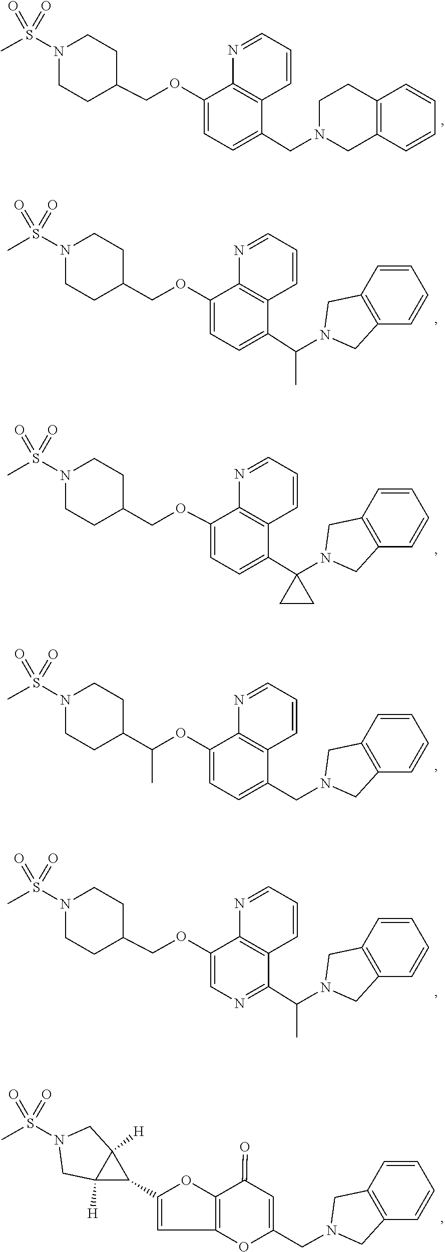 [SMILES: CS(=O)(=O)N1CCC(COc2ccc(CN3CCc4ccccc4C3)c3cccnc23)CC1 ; CC(c1ccc(OCC2CCN(S(C)(=O)=O)CC2)c2ncccc12)N1Cc2ccccc2C1 ; CS(=O)(=O)N1CCC(COc2ccc(C3(N4Cc5ccccc5C4)CC3)c3cccnc23)CC1 ; CC(Oc1ccc(CN2Cc3ccccc3C2)c2cccnc12)C1CCN(S(C)(=O)=O)CC1 ; CC(c1ncc(OCC2CCN(S(C)(=O)=O)CC2)c2ncccc12)N1Cc2ccccc2C1 ; CS(=O)(=O)N1C[C@@H]2[C@H](C1)[C@H]2c1cc2oc(CN3Cc4ccccc4C3)cc(=O)c2o1]

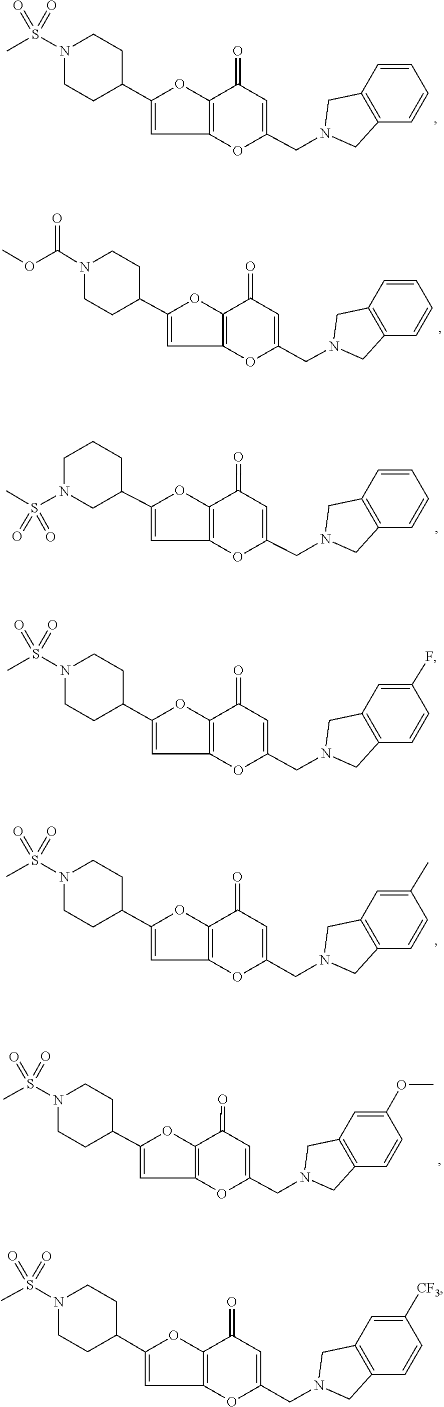

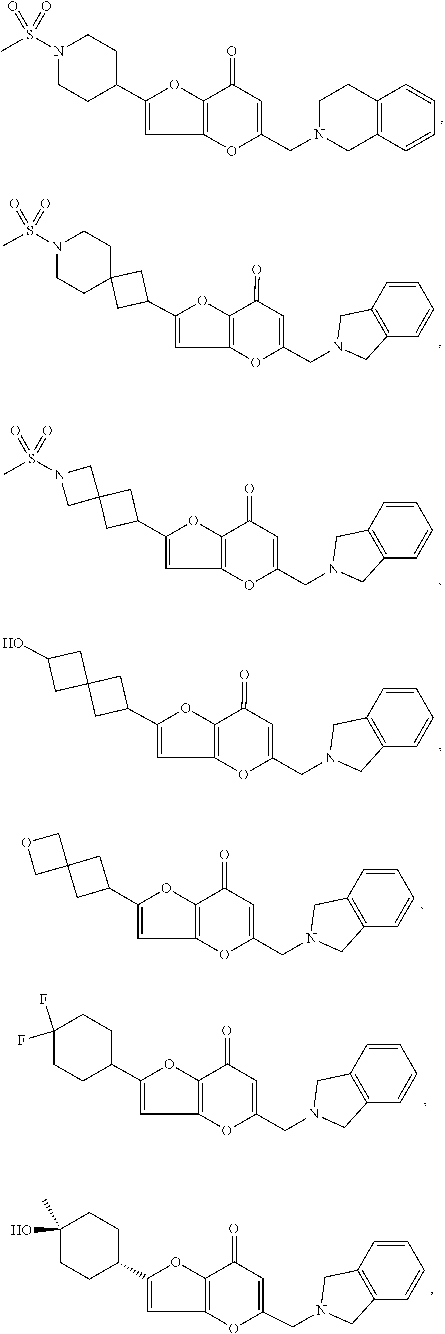

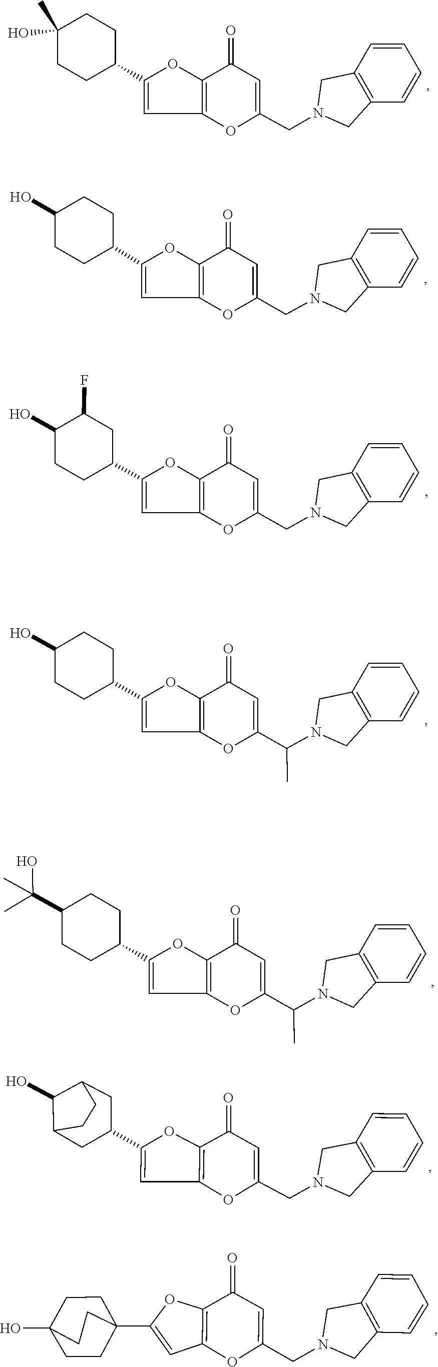

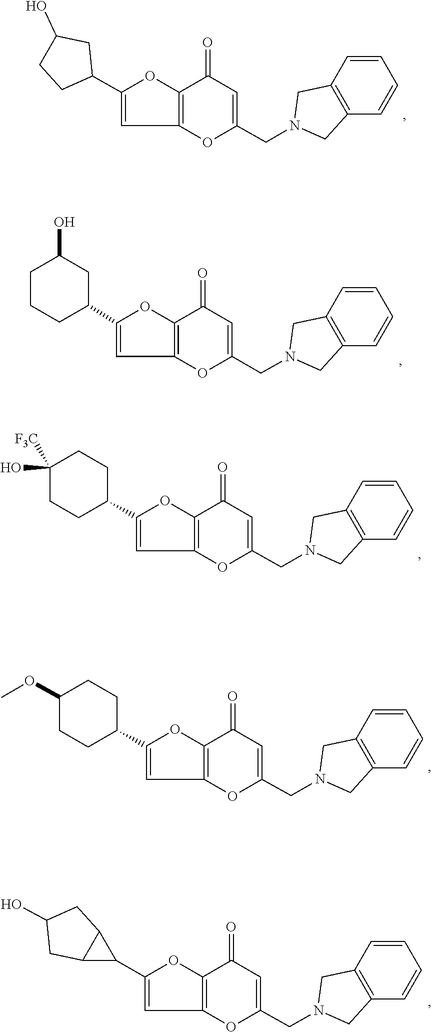 [SMILES: O=c1cc(CN2Cc3ccccc3C2)oc2cc(C3CCC(O)C3)oc12 ; O=c1cc(CN2Cc3ccccc3C2)oc2cc([C@@H]3CCC[C@@H](O)C3)oc12 ; O=c1cc(CN2Cc3ccccc3C2)oc2cc([C@H]3CC[C@@](O)(C(F)(F)F)CC3)oc12 ; CO[C@H]1CC[C@H](c2cc3oc(CN4Cc5ccccc5C4)cc(=O)c3o2)CC1 ; O=c1cc(CN2Cc3ccccc3C2)oc2cc(C3C4CC(O)CC43)oc12]

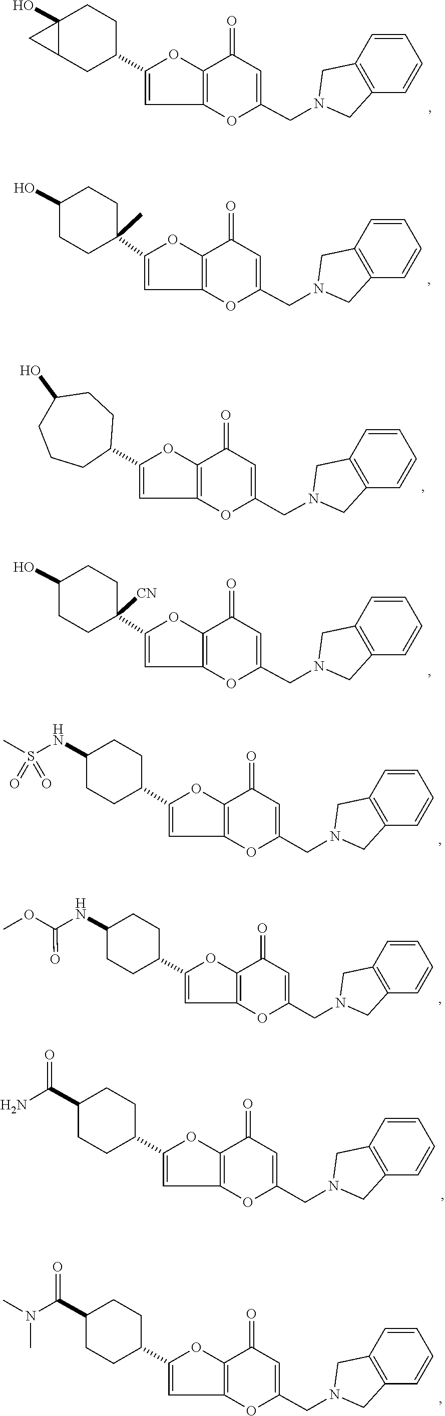

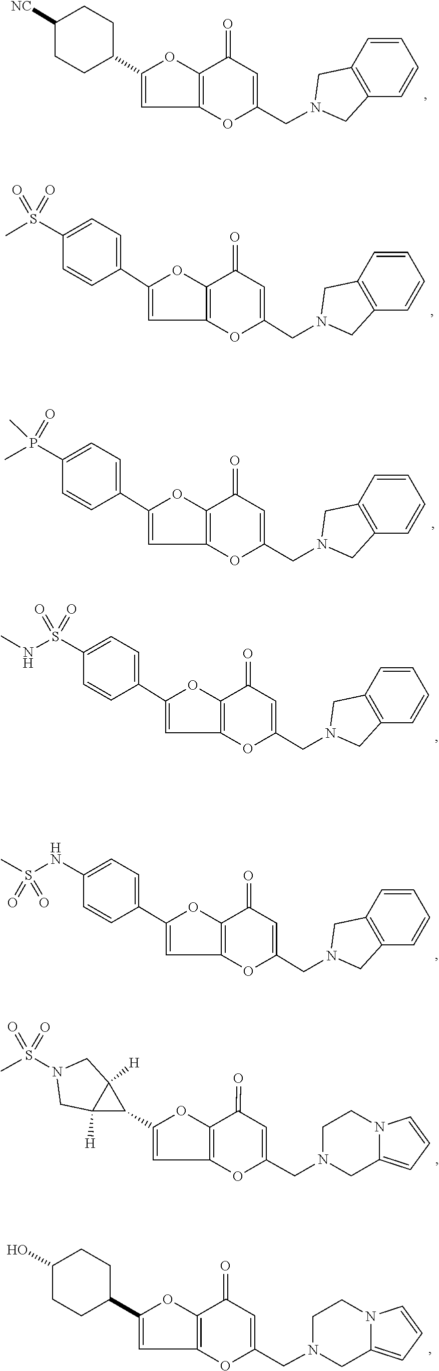

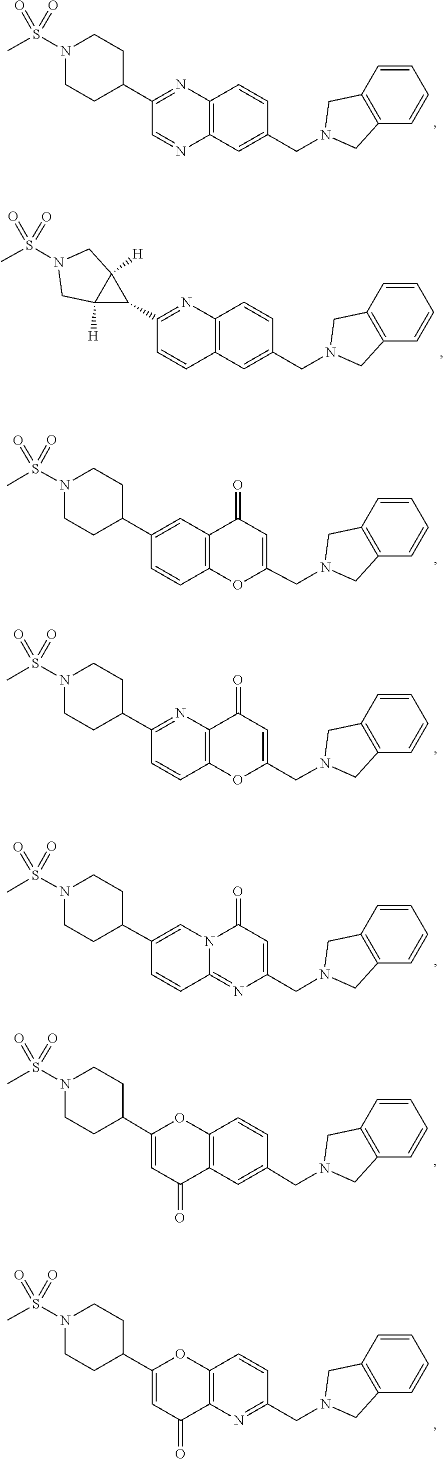

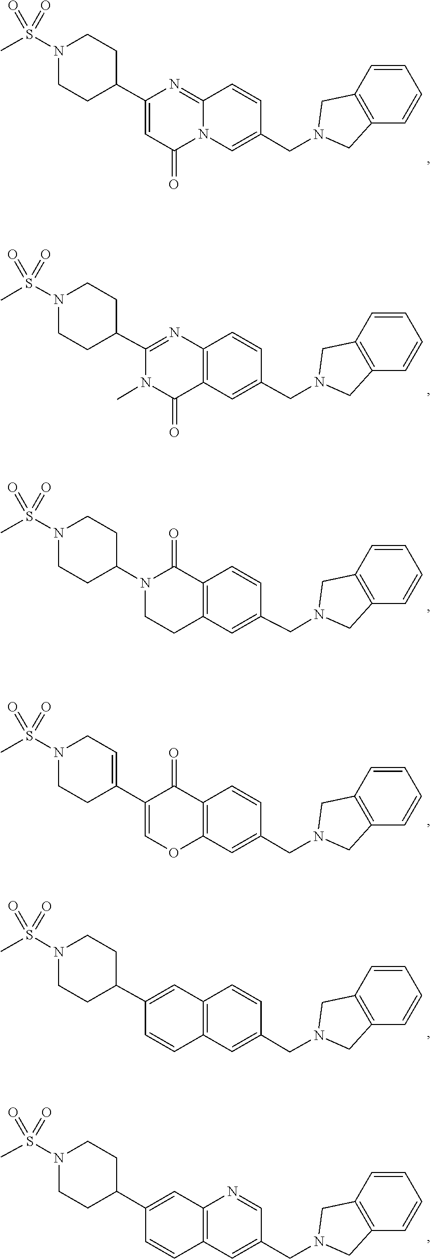 [SMILES: CS(=O)(=O)N1CCC(c2cc(=O)n3cc(CN4Cc5ccccc5C4)ccc3n2)CC1 ; Cn1c(C2CCN(S(C)(=O)=O)CC2)nc2ccc(CN3Cc4ccccc4C3)cc2c1=O ; CS(=O)(=O)N1CCC(N2CCc3cc(CN4Cc5ccccc5C4)ccc3C2=O)CC1 ; CS(=O)(=O)N1CC=C(c2coc3cc(CN4Cc5ccccc5C4)ccc3c2=O)CC1 ; CS(=O)(=O)N1CCC(c2ccc3cc(CN4Cc5ccccc5C4)ccc3c2)CC1 ; CS(=O)(=O)N1CCC(c2ccc3cc(CN4Cc5ccccc5C4)cnc3c2)CC1]

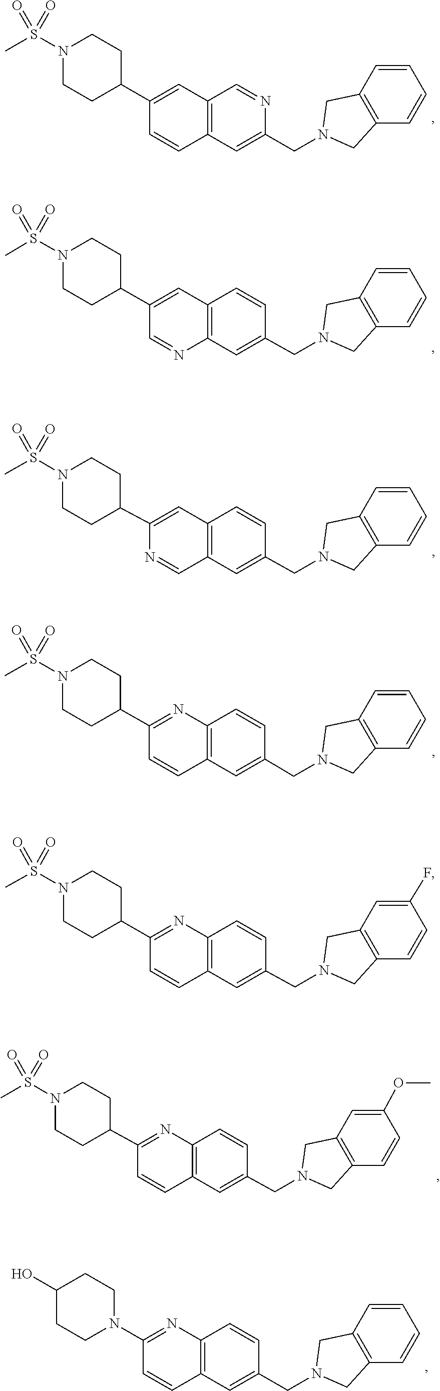

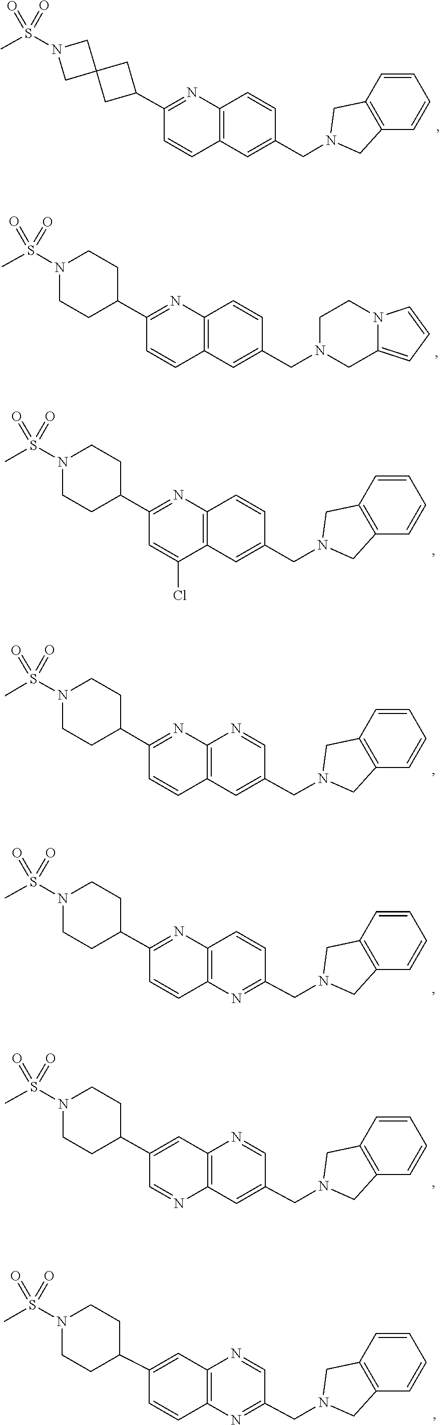

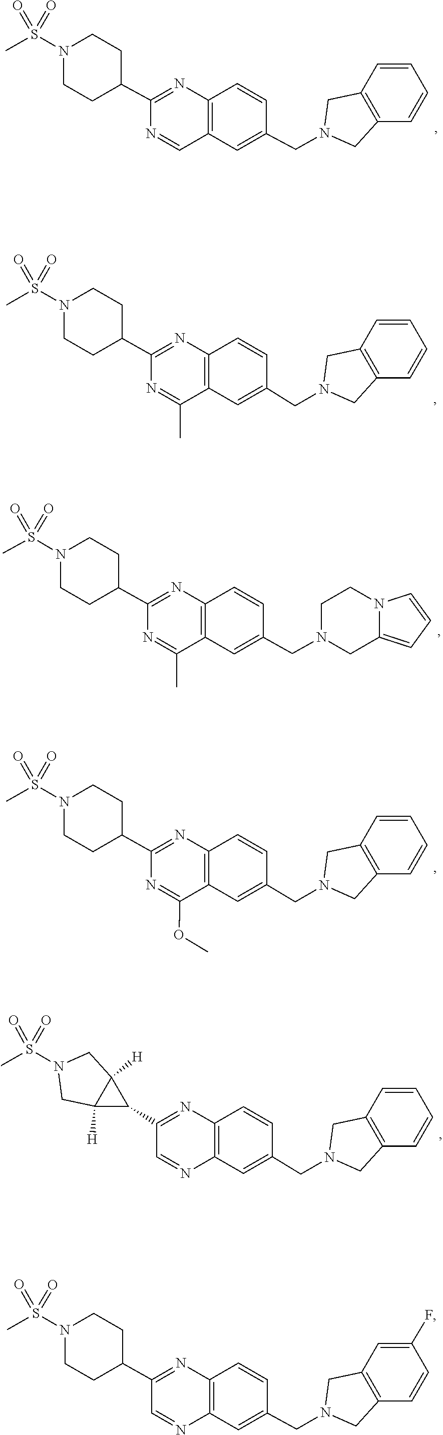 [SMILES: CS(=O)(=O)N1CCC(c2ncc3cc(CN4Cc5ccccc5C4)ccc3n2)CC1 ; Cc1nc(C2CCN(S(C)(=O)=O)CC2)nc2ccc(CN3Cc4ccccc4C3)cc12 ; Cc1nc(C2CCN(S(C)(=O)=O)CC2)nc2ccc(CN3CCn4cccc4C3)cc12 ; COc1nc(C2CCN(S(C)(=O)=O)CC2)nc2ccc(CN3Cc4ccccc4C3)cc12 ; CS(=O)(=O)N1C[C@@H]2[C@H](C1)[C@H]2c1cnc2cc(CN3Cc4ccccc4C3)ccc2n1 ; CS(=O)(=O)N1CCC(c2cnc3cc(CN4Cc5ccc(F)cc5C4)ccc3n2)CC1]

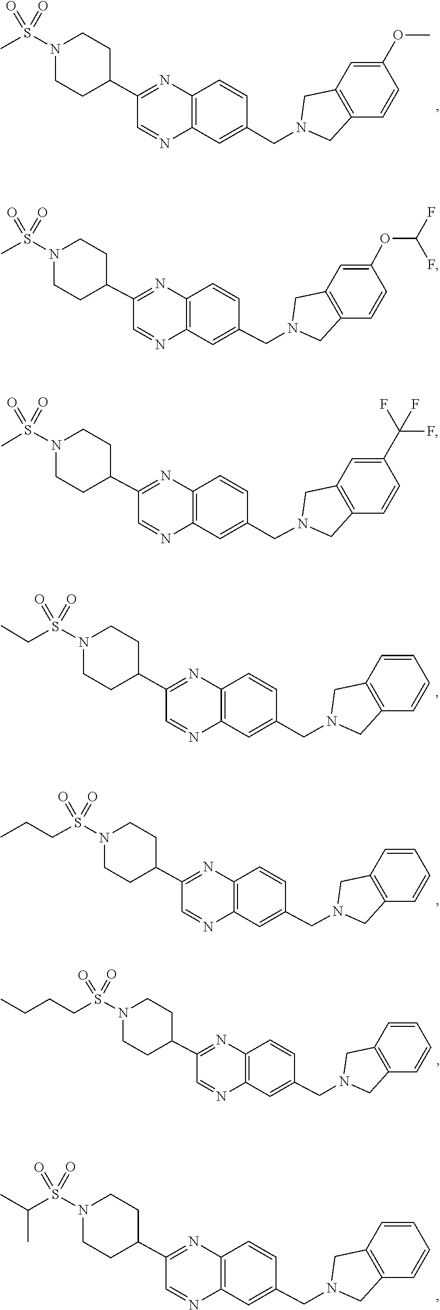

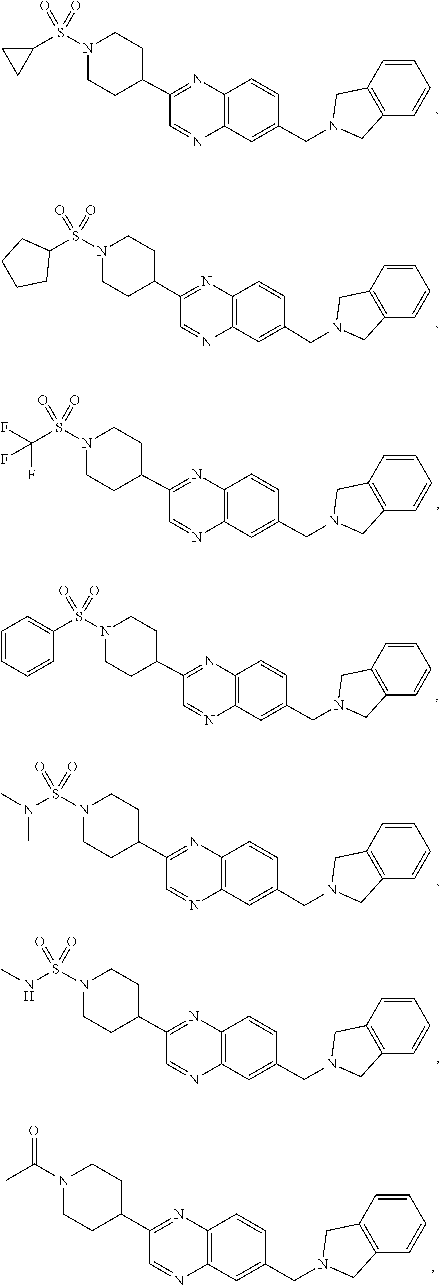

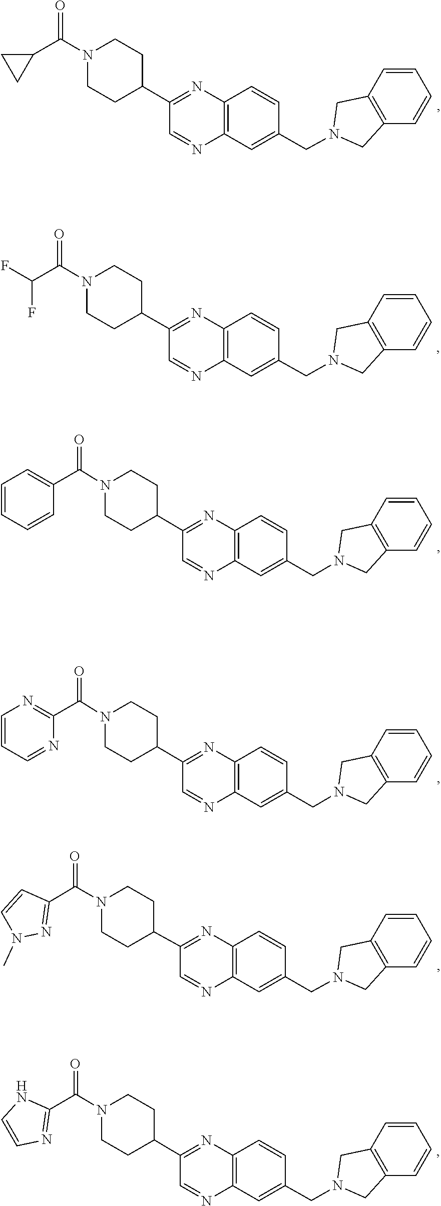 [SMILES: O=C(C1CC1)N1CCC(c2cnc3cc(CN4Cc5ccccc5C4)ccc3n2)CC1 ; O=C(C(F)F)N1CCC(c2cnc3cc(CN4Cc5ccccc5C4)ccc3n2)CC1 ; O=C(c1ccccc1)N1CCC(c2cnc3cc(CN4Cc5ccccc5C4)ccc3n2)CC1 ; O=C(c1ncccn1)N1CCC(c2cnc3cc(CN4Cc5ccccc5C4)ccc3n2)CC1 ; Cn1ccc(C(=O)N2CCC(c3cnc4cc(CN5Cc6ccccc6C5)ccc4n3)CC2)n1 ; O=C(c1ncc[nH]1)N1CCC(c2cnc3cc(CN4Cc5ccccc5C4)ccc3n2)CC1]

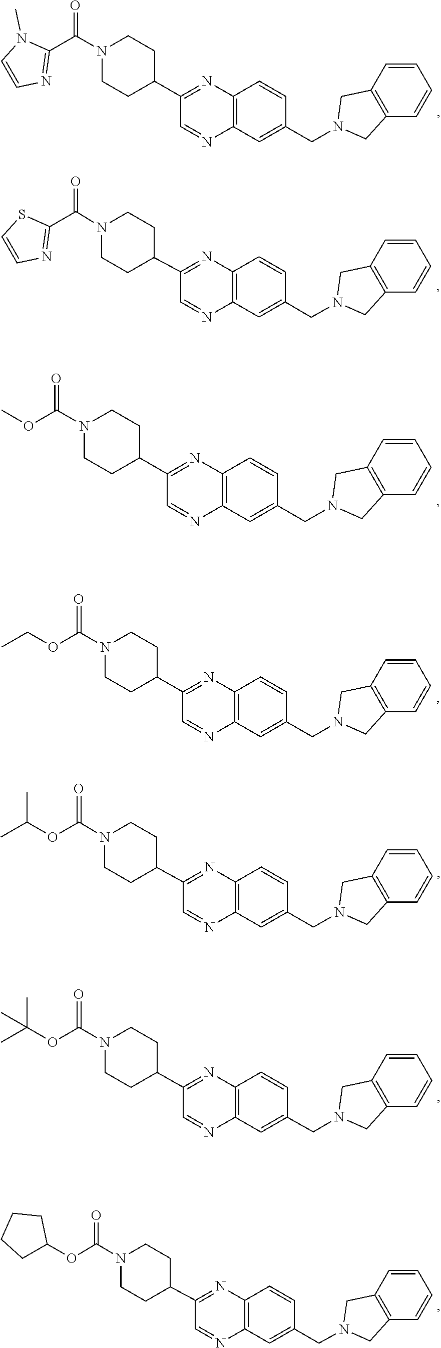

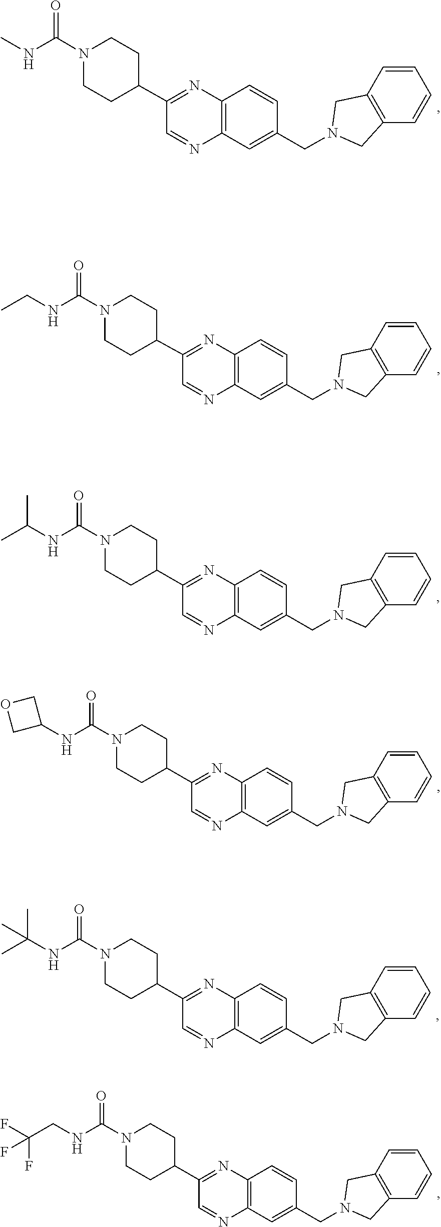 [SMILES: CNC(=O)N1CCC(c2cnc3cc(CN4Cc5ccccc5C4)ccc3n2)CC1 ; CCNC(=O)N1CCC(c2cnc3cc(CN4Cc5ccccc5C4)ccc3n2)CC1 ; CC(C)NC(=O)N1CCC(c2cnc3cc(CN4Cc5ccccc5C4)ccc3n2)CC1 ; O=C(NC1COC1)N1CCC(c2cnc3cc(CN4Cc5ccccc5C4)ccc3n2)CC1 ; CC(C)(C)NC(=O)N1CCC(c2cnc3cc(CN4Cc5ccccc5C4)ccc3n2)CC1 ; O=C(NCC(F)(F)F)N1CCC(c2cnc3cc(CN4Cc5ccccc5C4)ccc3n2)CC1]

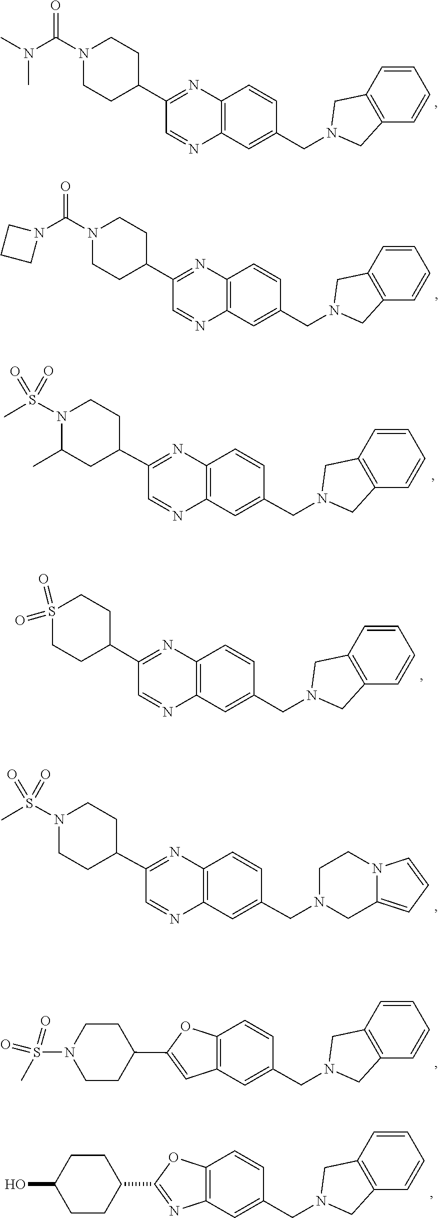

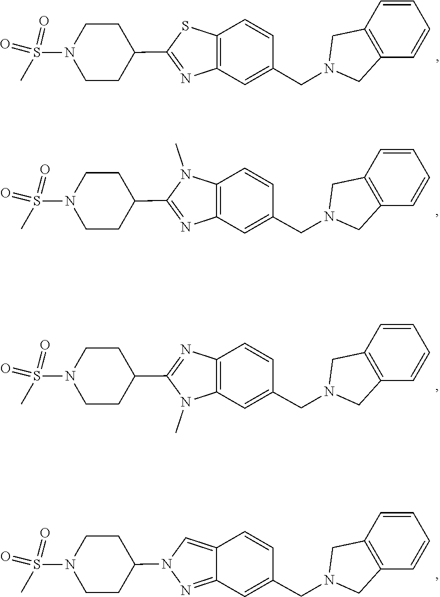 [SMILES: CS(=O)(=O)N1CCC(c2nc3cc(CN4Cc5ccccc5C4)ccc3s2)CC1 ; Cn1c(C2CCN(S(C)(=O)=O)CC2)nc2cc(CN3Cc4ccccc4C3)ccc21 ; Cn1c(C2CCN(S(C)(=O)=O)CC2)nc2ccc(CN3Cc4ccccc4C3)cc21 ; CS(=O)(=O)N1CCC(n2cc3ccc(CN4Cc5ccccc5C4)cc3n2)CC1]

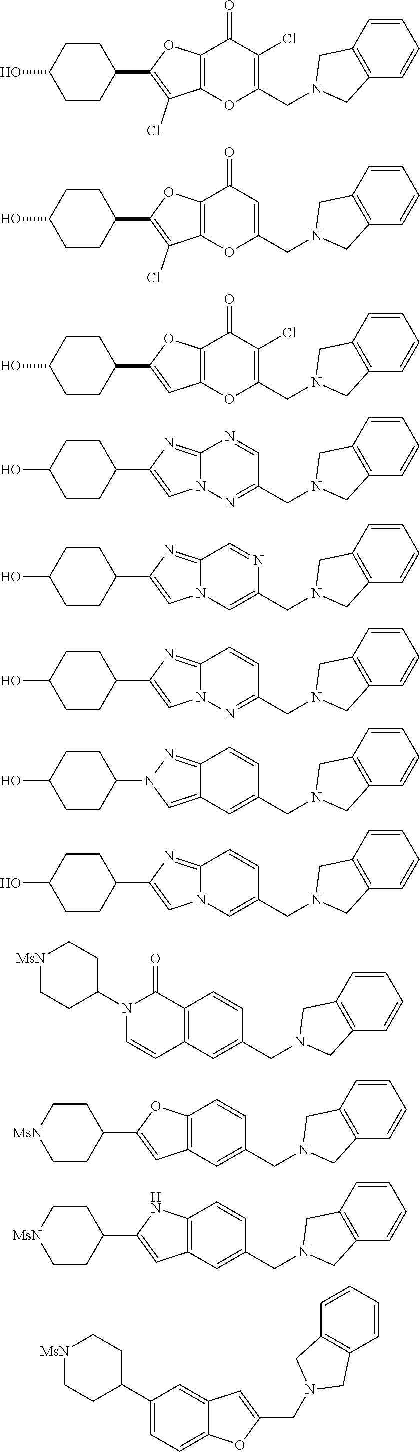

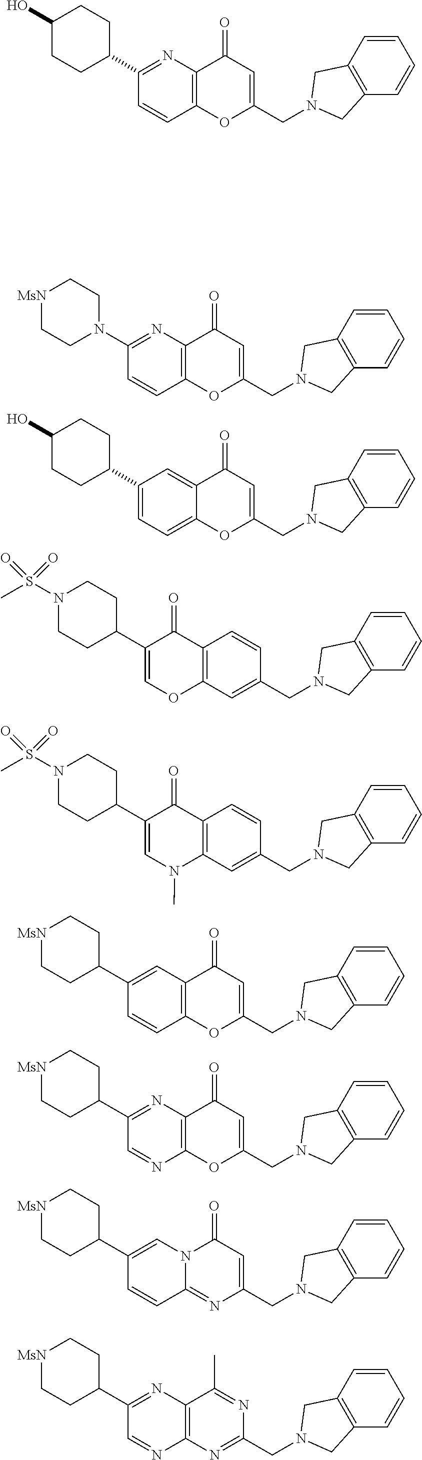

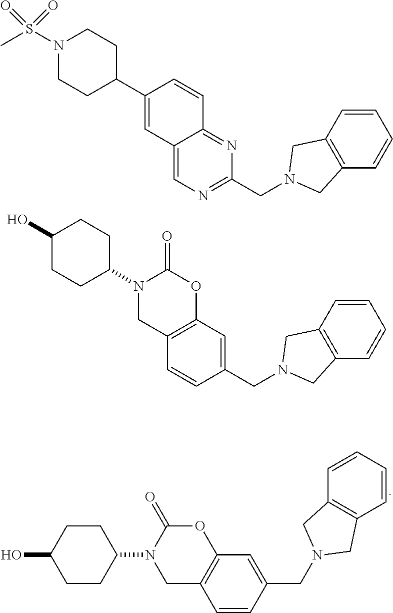

In some embodiments, the compounds according to the present invention have CYP11A1 inhibitory effect.

Another aspect of this invention includes a pharmaceutical composition, each including a therapeutically effective amount of the compound as described, a pharmaceutically acceptable salt, tautomer, deuterated ones, or stereoisomer thereof, in combination with one or more physiologically acceptable carriers or excipients.

Such compounds or pharmaceutical composition according to the present invention are useful in the treatment of steroid hormone receptor dependent conditions and diseases. In some embodiments, the steroid hormone receptor dependent conditions and diseases are cancers, including, but not limited to, prostate cancer and breast cancer. In some embodiments, the steroid hormone receptor is AR, and the AR dependent disease is prostate cancer. In some embodiments, the prostate cancer is castration-resistant prostate cancer (CRPC).

In some other embodiments, such pharmaceutical composition may include a second therapeutic agent.

Yet still another aspect of this invention provides a method for treating a steroid hormone receptor dependent condition or disease in a subject in need thereof, comprising administering to the subject in need thereof an effective amount of the compound or the pharmaceutical composition as described.

Yet still another aspect of this invention provides use of the compounds as described for the manufacture of a medicament for treating conditions and diseases mediated by steroid hormone receptor.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications, and equivalents, which may be included within the spirit and scope of the invention as defined by the claims. Furthermore, in the detailed description of the present invention, numerous specific details are set forth to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

### Definitions

Unless the context indicates otherwise, references to Formula (I) in all sections of this document (including the uses, methods and other aspects of the invention) include references to all other sub-formula, sub-groups, preferences, embodiments and examples as defined herein.

Unless otherwise stated, the following terms used in the specification and claims have the meanings discussed below:

As used herein, the term “or” is meant to include both “and” and “or”. In other words, the term “or” may also be replaced with “and/or”.

In defining various terms, for example, “X”, “Y₁” and “Z₁”, are used herein as generic symbols to represent various specific chemical elements.

A dashed line represents a single bond or a double bond as required to complete the valences of the atoms being linked by the bond.

As used herein, the term “unsaturated bond” refers to a double or triple bond.

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” by itself or as part of another substituent refers to a linear (i.e., unbranched or straight) or branched hydrocarbon chain radical consisting of carbon and hydrogen atoms, containing no unsaturation, having the stated number of carbon atoms (e.g., C₁-C₁₀ or C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated. Representative saturated linear or straight alkyl includes, but not limited to, -methyl, -ethyl, -n-propyl, -n-butyl, -n-pentyl, and -n-hexyl; while saturated branched alkyl includes, but not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more substituents.

When any H in “alkyl” is replaced with deuterium (D), the alkyl is a “deuterated alkyl”.

The term “alkylene” by itself or as part of another molecule means a divalent radical derived from an alkane, which can be a straight chain or branched chain. In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, refers to an alkylene group having from 1 to 4 carbon atoms. Examples of linear C₁₋₈ alkylene groups include, but are not limited to, —(CH₂)ₙ— where n is an integer from 1 to 7, for example, —CH₂—, —CH₂CH₂CH₂—, and —CH₂CH₂CH₂CH₂—. Examples of branched C₁₋₇alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

As used herein, the term “alkenyl” by itself or as part of another substituent refers to an unsaturated branched or straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl, propenyl, and the like.

As used herein, the term “alkynyl” by itself or as part of another substituent refers to carbon chains which contain at least one carbon-carbon triple bond, and which may be linear or branched or combinations thereof. Examples of alkynyl include ethynyl, propargyl, 3-methyl-1-pentynyl, 2-heptynyl and the like.

As used herein, the term “cycloalkyl” by itself or as part of another substituent refers to a non-aromatic carbon-based ring composed of at least three carbon atoms. The term cycloalkyl includes monocyclic cycloalkyl, bicyclic cycloalkyl, polycyclic cycloalkyl, bridged cycloalkyl, fused cycloalkyl, and spiro cycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.

The term “cycloalkenyl” as used herein is a non-aromatic carbon-based ring composed of at least three carbon atoms and containing at least one carbon-carbon double bond, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, wherein at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted.

The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

The term “heterocycle” or “heterocyclyl” as used herein, refers to a group derived from a monocyclic, bridged bicyclic, fused bicyclic, spirocyclic or polycyclic moiety comprising at least one nonaromatic ring comprising one or more ring-forming heteroatoms independently selected from nitrogen, oxygen, and sulfur. The nitrogen atom may be substituted or unsubstituted (i.e., N or NR wherein R is H or another substituent, if defined). The heterocyclyl can be unsaturated, saturated, or partially unsaturated. In certain embodiments, a heterocyclyl may comprises 1 to 4 heteroatoms as ring members. The heterocyclyl groups of the present disclosure can be attached to the parent molecular moiety through a carbon atom or a heteroatom in the group. Thus, the term is inclusive of, but not limited to, “heterocycloalkyl”, “heteroaryl”, “bicyclic heterocycle” and “polycyclic heterocycle”.

The term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can be monocyclic or polycyclic.

As used herein, the term “halo” or “halogen” refers to fluorine (fluoro, —F), chlorine (chloro, —Cl), bromine (bromo, —Br), or iodine (iodo, —I).

The term “haloalkyl” refers to alkyl as defined above in which one or more of the hydrogen atoms have been replaced with a halogen independently selected from fluoro, chloro, bromo, and iodo. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be —CH₂CF₃, —CHF—CH₃, or —CH₂CH₂F.

As used herein, the term “hydrogen” (or H) includes its isotopes of deuterium (D or ²H) and tritium (³H), meaning a or any hydrogen atom in the compounds of this invention can be replaced with either deuterium (D or ²H) and tritium (³H).

As used herein, the term “alkoxy” or “alkoxyl” refers to a saturated straight or branched hydrocarbon linked to an oxygen atom. Representative saturated straight chain alkoxys include methoxy, ethoxy, n-propoxy, n-butoxy, n-pentoxy, n-hextoxy, and the like; while saturated branched alkoxys include isopropoxyl, sec-butoxy, isobutoxy, tert-butoxy, isopentoxy, and the like. Cyclic alkoxy are referred to herein as a “cycloalkoxy”. “C₁₋₄alkoxy” refers to an alkyl with 1, 2, 3, or 4 carbon atoms. Alkoxy can be linked to a molecule by one or two attachment points.

As used herein, the term “haloalkoxy” refers to an alkoxy group substituted with 1 to 6 halo groups.

The term “alkoxyalkyl”, as used herein, refers to an alkyl group substituted with one, two, or three alkoxy groups.

As used herein, the term “aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The “aryl” group can be substituted or unsubstituted.

As used herein, the term “heteroaryl” refers to a monocyclic or fused ring (i.e., rings which share an adjacent pair of atoms) of 5 to 12 ring atoms containing one, two, three or four ring heteroatoms selected from N, O or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzothiazole, indazole and quinazoline. The heteroaryl group may be substituted or unsubstituted.

The term “arylene,” as used herein, refers to a bidentate moiety obtained by removing two hydrogen atoms, one from each of two different aromatic ring atoms of an aromatic compound, which moiety has from 3 to 20 ring atoms (unless otherwise specified). Preferably, each ring has from 5 to 7 ring atoms.

As used herein, the term “hydroxyl” or “hydroxy” refers to the group —OH.

The term “hydroxyalkyl” by itself or as part of another substituent refers to an alkyl group in which one or more of the hydrogen atoms are replaced with a hydroxyl substituent. Thus, the term “hydroxyalkyl” is meant to include monohydroxyalkyl, dihydroxyalkyl, trihydroxyalkyl, etc. In “hydroxyalkyl” group, the alkyl can be a linear (i.e., straight or unbranched) or branched alkyl, accordingly, the “hydroxyalkyl” group includes linear hydroxyalkyl and branched hydroxyalkyl.

The term “cyano” or “—CN” as used herein refers to a group of —C≡N. The term “cyanoalkyl” or “-alk-CN” as used herein, refers to an alkyl group having at least one —CN substituent. In general, if a compound is attached to an “-alk-CN” group, the alkylene portion of the “-alk-CN” group is attached to the compound. In “cyanoalkyl” group, the alkyl can be a linear (i.e., straight or unbranched) or branched alkyl, accordingly, the “cyanoalkyl” group includes linear cyanoalkyl and branched cyanoalkyl.

As used herein, the term “amino” or “amine” as used herein refers to —NH₂. The term “alkylamino” refers to a group of the formula —NHR, and “dialkylamino” refers to a group of the formula —NRR′, where each of R and R′ is independently an alkyl.

As used herein, the term “—SO₂—” or “—S(═O)₂—” refers to sulfur dioxide, having a structure of

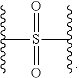 [SMILES: *S(*)(=O)=O]

As used herein, the term “carbonyl” or “—C(═O)—” or “—CO—” refers to a group having a structure of

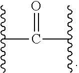 [SMILES: *C(*)=O]

As used herein, the term “—CO₂—” or “—COO—” refers to a group having a structure of

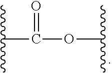 [SMILES: *OC(*)=O]

The above-defined groups may include prefixes and/or suffixes that are commonly used in the art to create additional well-recognized substituent groups. As examples, the term “haloalkoxy” or “(haloalkyl)oxy” refers to a haloalkyl group attached to the parent molecular moiety through an oxygen atom. The term “(haloalkyl)oxyalkyl” refers to an alkyl group substituted with one, two, or three (haloalkyl)oxy groups.

As used herein, the term “absent” that defines a variable, e.g., “X”, means that the defined variable is not present, and thus the two groups that connected through the variable are directly connected to each other.

As used herein, the term “oxo” (alone or in combination with another term) means (═O).

As sued herein, the term “bond” or “a bond” refers to a covalent linkage between two atoms or two moieties, which may indicate a single bond, a double bond, or a triple bond.

The term “stereoisomer” refers to isomers of identical constitution that differ only in spatial arrangement of atoms, rather than order of atomic connectivity. When a disclosed compound is named or depicted by structure without indicating stereochemistry, it is understood that the name or structure encompasses all possible stereoisomers, including essentially pure stereoisomers, as well as combination thereof. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The term “chiral” refers to the structural characteristic of a molecule that makes it impossible to superimpose it on its mirror image.

As used herein, the term “tautomer” refers to each of two or more isomers of a compound that exist together in equilibrium and are readily interchanged by migration of an atom or group within the molecule. Thus, this disclosure is intended to cover all possible tautomer even when a structure depicts only one of them.

The term “Optional” or “Optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

The term “pharmaceutically acceptable salts” refers to salts prepared from pharmaceutically acceptable non-toxic bases or acids including inorganic or organic bases and inorganic or organic acids, which are, within the scope of sound medical judgment, suitable for use in contact with the tissues of patients without excessive toxicity, irritation, allergic response, or other problem or complication commensurate with a reasonable benefit/risk ratio, and are effective for their intended use. Salts derived from inorganic bases include aluminum, ammonium, calcium, copper, ferric, ferrous, lithium, magnesium, manganic salts, manganous, potassium, sodium, zinc, and the like. Particularly preferred are ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methyl-glucamine, morpholine, piperazine, piperidine, polyamine resins, procaine, purines, theobromine, triethylamine, trimethylamine, tripropylamine, tromethamine, and the like.

When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula (I) are meant to also include the pharmaceutically acceptable salts.

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “therapeutically effective amount” refers to the amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

As used herein, the term “subject” or “patient” is used interchangeably and refers to any animal subject, including but not limited to human beings, laboratory animals (e.g., primates, rats, mice), livestock (e.g., cows, sheep, goats, pigs, turkeys, and chickens), and household pets (e.g., dogs, cats, and rodents).

The compounds taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be done by continuous infusion over a selected period of time.

### Isomeric Forms

The present invention provides novel compounds of Formula (I), or a pharmaceutically acceptable salt therefore, as CYP11A1 inhibitors.

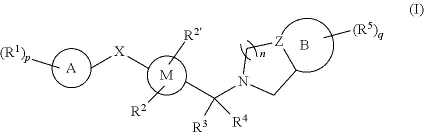

It will be appreciated that certain compounds (or salts, prodrugs, or conjugates) of the present invention may exist in, and be isolated in, isomeric forms, including tautomeric forms, geometric isomers (i.e., cis- or trans-isomers), optical isomers (i.e., enantiomers and diastereomers), racemic forms, or any mixture of the isomeric forms described above. It is to be understood that the present invention encompasses a compound of Formula (I) in any of the isomeric forms or as a mixture thereof, for example, in the form of an active single enantiomer, racemic, or any mixture thereof. The present invention is meant to comprehend all such isomeric forms of the compounds of Formula (I).

In addition, the compound (or salt, prodrug or conjugate thereof) of the present invention may exhibit polymorphism or may form a solvate with water or an organic solvent. The present invention also encompasses any such polymorphic form, any solvate or any mixture thereof.

### Example 1: 2-(isoindolin-2-ylmethyl)-5-(((1R,5S,6r)-3-(methylsulfonyl)-3-azabicyclo[3.1.0]hexan-6-yl)methoxy)-4H-pyran-4-one

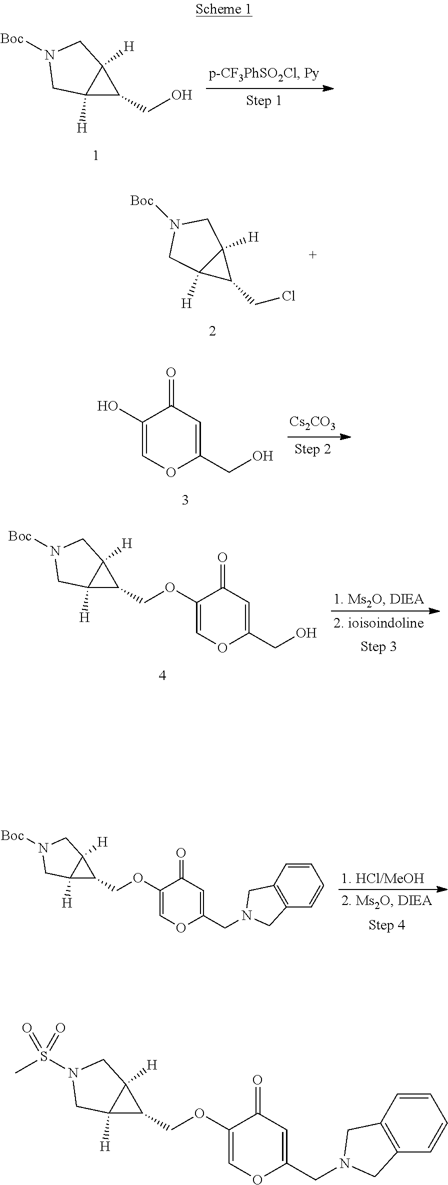

Step 1: To a solution of 1 (2.0 g, 1.0 eq) in DCM (20 mL) was added pyridine (2.0 mL, 2.6 eq) and p-CF₃PhSO₂Cl (2.8 g, 1.2 eq). The mixture was stirred at 25° C. for 16 h. The reaction was purified by flash chromatography to give 2 (1.3 g, 60% yield) as a colorless oil.

¹H NMR (400 MHz, CDCl₃) δ 3.70-3.39 (m, 4H), 3.38-3.27 (m, 2H), 1.59-1.50 (m, 2H), 1.49-1.35 (s, 9H), 1.18-1.04 (m, 1H).

Step 2: To a solution of 2 (1.0 g, 1.0 eq) in DMA (10 mL) was added Cs₂CO₃ (2.8 g, 2.0 eq) and 3 (1.2 g, 2.0 eq). The mixture was stirred at 70° C. for 16 h. The reaction was purified by flash chromatography to give 4 (630 mg, 43% yield) as a colorless oil.

MS m/z: 338.1 (M+1)⁺.

¹H NMR (400 MHz, CDCl₃) δ 7.75-7.51 (m, 1H), 6.74-6.28 (m, 1H), 4.57-4.40 (m, 2H), 4.00-3.86 (m, 1H), 3.85-3.73 (m, 1H), 3.69-3.55 (m, 2H), 3.43-3.30 (m, 2H), 3.00 (br s, 1H), 1.61-1.50 (m, 2H), 1.50-1.37 (m, 9H), 1.19-0.98 (m, 1H).

Step 3: To a solution of 4 (140 mg, 1.0 eq) in DCM (5 mL) was added DIEA (160 mg, 3.0 eq) and methylsulfonyl methanesulfonate (143 mg, 2.0 eq). The mixture was stirred at 20° C. for 0.5 hr. The reaction was concentrated and dissolved in CH₃CN (3 mL) was added DIEA (72 μL, 1.0 eq) and ioisoindoline (132 mg, 2.0 eq). The mixture was stirred at 20° C. for 2 h. The reaction was purified by flash chromatography to give 5. MS m/z: 439.2 (M+1)⁺.

Step 4: 5 (150 mg, 1.0 eq) was dissolved in HCl/MeOH (2 M, 1.7 mL). The mixture was stirred at 20° C. for 0.5 hr. The reaction was concentrated and dissolved in DCM (5 mL) was added DIEA (293 μL, 5.0 eq) and Ms₂O (117 mg, 2.0 eq). The mixture was stirred at 20° C. for 0.5 h. The reaction was purified to afford 2-(isoindolin-2-ylmethyl)-5-(((1R,5S,6r)-3-(methylsulfonyl)-3-azabicyclo[3.1.0]hexan-6-yl)methoxy)-4H-pyran-4-one. MS m/z: 417.1 (M+1)⁺.

¹H NMR (400 MHz, CD₃OD) δ 8.15 (s, 1H), 7.43 (s, 4H), 6.74 (s, 1H), 4.82 (br s, 4H), 4.68 (s, 2H), 3.89 (d, J=7.2 Hz, 2H), 3.53 (d, J=9.2 Hz, 2H), 3.39 (d, J=9.2 Hz, 2H), 2.88 (s, 3H), 1.73 (br s, 2H), 1.40-1.36 (m, 1H).

Examples 2-13 were synthesized using similar methods as that of Example 1.

| Example | | |
|---|---|---|
| No. | Structure | Mass and ¹HNMR |
| 2 | 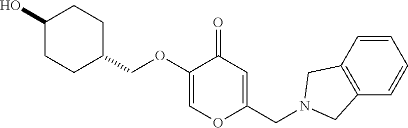 [SMILES: O=c1cc(CN2Cc3ccccc3C2)occ1OC[C@H]1CC[C@H](O)CC1] | MS m/z: 356.2 (M + 1)⁺. ¹H NMR (400 MHz, CD₃OD) δ 8.16 (s, 1H), 7.42 (s, 4H), 6.77 (s, 1H), 4.85 (br s, 4H), 4.72 (s, 2H), 3.74 (d, J = 6.4 Hz, 2H), 3.58-3.48 (m, 1H), 2.03-1.91 (m, 4H), 1.83- 1.71 (m, 1H), 1.37-1.25 (m, 2H), 1.21-1.07 (m, 2H). |
| 3 | 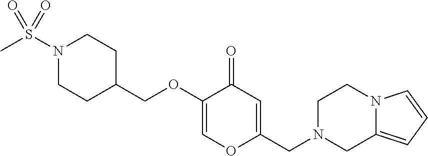 [SMILES: CS(=O)(=O)N1CCC(COc2coc(CN3CCn4cccc4C3)cc2=O)CC1] | MS m/z: 422.2 (M + 1)⁺. ¹H NMR (400 MHz, CDCl₃) δ 7.58 (s, 1H), 6.56 (dd, J = 2.8, 1.6 Hz, 1H), 6.49 (s, 1H), 6.16-6.12 (m, 1H), 5.83 (dd, J = 3.6, 1.6 Hz, 1H), 4.04-3.99 (m, 2H), 3.89-3.82 (m, 2H), 3.76-3.71 (m, 4H), 3.55 (s, 2H), 2.97-2.91 (m, 2H), 2.79 (s, 3H), 2.68 (td, J = 12.0, 2.4 Hz, 2H), 2.05-1.96 (m, 3H), 1.48-1.36 (m, 2H). |
| 4 | 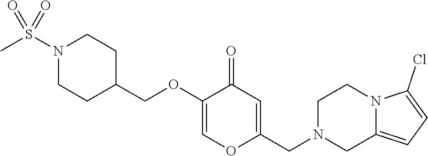 [SMILES: CS(=O)(=O)N1CCC(COc2coc(CN3CCn4c(Cl)ccc4C3)cc2=O)CC1] | MS m/z: 456.2 (M + 1)⁺. ¹H NMR (400 MHz, CDCl₃) δ 7.59 (s, 1H), 6.48 (s, 1H), 6.00 (d, J = 3.6 Hz, 1H), 5.81 (d, J = 3.6 Hz, 1H), 3.94-3.88 (m, 2H), 3.88- 3.81 (m, 2H), 3.73 (d, J = 6.0 Hz, 2H), 3.68 (s, 2H), 3.55 (s, 2H), 2.96 (dd, J = 6.4, 4.8 Hz, 2H), 2.79 (s, 3H), 2.69 (td, J = 12.0, 2.4 Hz, 2H), 2.10-1.90 (m, 3H), 1.48-1.36 (m, 2H). |
| 5 | 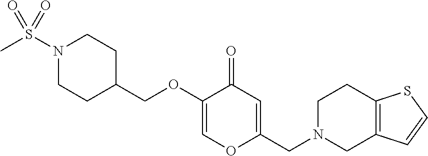 [SMILES: CS(=O)(=O)N1CCC(COc2coc(CN3CCc4sccc4C3)cc2=O)CC1] | MS m/z: 439.2 (M + 1)⁺. ¹H NMR (400 MHz, CDCl₃) δ 7.60 (s, 1H), 7.10 (d, J = 5.2 Hz, 1H), 6.71 (d, J = 5.2 Hz, 1H), 6.49 (s, 1H), 3.85 (d, J = 12.0 Hz, 2H), 3.73 (d, J = 6.0 Hz, 2H), 3.62 (d, J = 25.2 Hz, 4H), 2.92 (s, 4H), 2.79 (s, 3H), 2.66 (s, 2H), 2.23- 1.89 (m, 3H), 1.48-1.37 (m, 2H). |
| 6 | 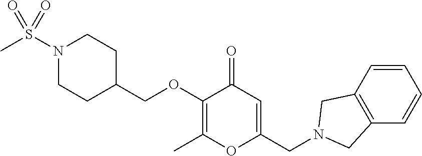 [SMILES: Cc1oc(CN2Cc3ccccc3C2)cc(=O)c1OCC1CCN(S(C)(=O)=O)CC1] | MS m/z: 433.2 (M + 1)⁺. ¹H NMR (400 MHz, CD₃OD) δ 7.23 (s, 4H), 6.48 (s, 1H), 4.04 (s, 4H), 3.91-3.82 (m, 4H), 3.76 (d, J = 11.6 Hz, 2H), 2.86- 2.74 (m, 5H), 2.39 (s, 3H), 2.09-1.92 (m, 3H), 1.51-1.38 (m, 2H). |
| 7 | 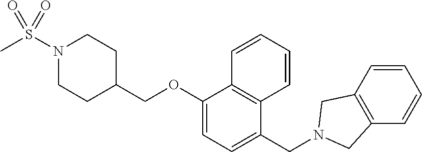 [SMILES: CS(=O)(=O)N1CCC(COc2ccc(CN3Cc4ccccc4C3)c3ccccc23)CC1] | MS m/z: 451.2 (M + 1)⁺. ¹H NMR (400 MHz, DMSO-d6) δ 8.36 (d, J = 8.4 Hz, 1H), 8.31 (dd, J = 8.4, 1.6 Hz, 1H), 7.84 (d, J = 8.0 Hz, 1H), 7.75- 7.66 (m, 1H), 7.66-7.59 (m, 1H), 7.41-7.30 (m, 4H), 7.10 (d, J = 8.0 Hz, 1H), 5.04 (d, J = 5.6 Hz, 2H), 4.71 (s, 4H), 4.13 (s, 2H), 3.66 (d, J = 11.6 Hz, 2H), 2.90 (s, 3H), 2.85-2.75 (m, 2H), 2.15-1.92 (m, 3H), 1.60-1.38 (m, 2H). |
| 8 | 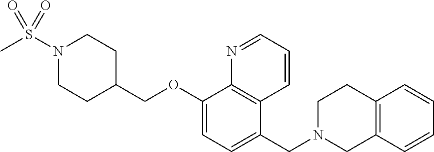 [SMILES: CS(=O)(=O)N1CCC(COc2ccc(CN3CCc4ccccc4C3)c3cccnc23)CC1] | MS m/z: 466.6 (M + 1)⁺. ¹H NMR (400 MHz, CDCl₃) δ 8.93 (dd, J = 1.6, 4.0 Hz, 1H), 8.73 (dd, J = 1.6, 8.4 Hz, 1H), 7.45-7.35 (m, 2H), 7.17-7.06 (m, 3H), 7.01-6.94 (m, 2H), 4.10 (d, J = 6.8 Hz, 2H), 3.98 (s, 2H), 3.90 (d, J = 11.6 Hz, 2H), 3.66 (s, 2H), 2.87- 2.82 (m, 2H), 2.81 (s, 3H), 2.79-2.71 (m, 4H), 2.35-2.23 (m, 1H), 2.16 (d, J = 13.2 Hz, 2H), 1.56- 1.46 (m, 2H). |
| 9 | 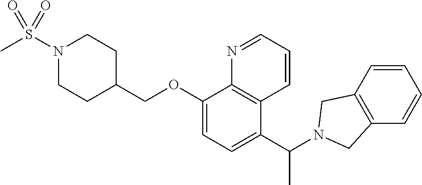 [SMILES: CC(c1ccc(OCC2CCN(S(C)(=O)=O)CC2)c2ncccc12)N1Cc2ccccc2C1] | MS m/z: 466.2 (M + 1)⁺. ¹H NMR (400 MHz, CD₃OD) δ 9.54 (d, J = 8.8 Hz, 1H), 9.22 (d, J = 4.4 Hz, 1H), 8.32 (d, J = 8.4 Hz, 1H), 8.22 (dd, J = 5.2, 8.4 Hz, 1H), 7.80 (d, J = 8.4 Hz, 1H), 7.51-7.16 (m, 4H), 5.82 (q, J = 6.8 Hz, 1H), 5.15 (br s, 2H), 4.38 (d, J = 6.4 Hz, 4H), 3.84 (d, J = 11.6 Hz, 2H), 2.91-2.81 (m, 5H), 2.28 (br s, 1H), 2.15 (d, J = 11.2 Hz, 2H), 1.99 (d, J = 6.4 Hz, 3H), 1.66-1.55 (m, 2H). |
| 10 | 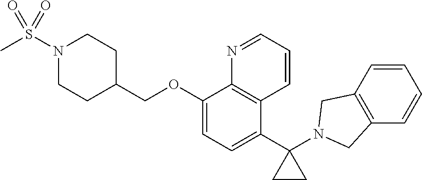 [SMILES: CS(=O)(=O)N1CCC(COc2ccc(C3(N4Cc5ccccc5C4)CC3)c3cccnc23)CC1] | MS m/z: 478.1 (M + 1)⁺. ¹H NMR (400 MHz, CD₃OD) δ 9.84-9.82 (m, 1H), 9.26 (dd, J = 1.2, 5.2 Hz, 1H), 8.39-8.32 (m, 2H), 7.75 (d, J = 8.4 Hz, 1H), 7.29-7.26 (m, 4H), 4.86-4.71 (m, 4H), 4.38 (d, J = 6.4 Hz, 2H), 3.82 (d, J = 11.6 Hz, 2H), 2.87-2.82 (m, 5H), 2.37-2.24 (m, 1H), 2.13-2.10 (m, 4H), 1.61-1.53 (m, 4H). |
| 11 | 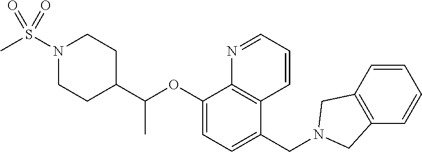 [SMILES: CC(Oc1ccc(CN2Cc3ccccc3C2)c2cccnc12)C1CCN(S(C)(=O)=O)CC1] | MS m/z: 466.2 (M + H)⁺. ¹H NMR (400 MHz, CDCl₃) δ 8.93 (dd, J = 1.6, 4.0 Hz, 1H), 8.71 (dd, J = 1.6, 8.4 Hz, 1H), 7.47-7.38 (m, 2H), 7.17 (s, 4H), 6.98 (d, J = 8.0 Hz, 1H), 4.42 (m, 1H), 4.22 (s, 2H), 3.93 (s, 4H), 3.92-3.81 (m, 2H), 2.79 (s, 3H), 2.69 (dt, J = 2.4, 12.0 Hz, 2H), 2.31 (d, J = 13.2 Hz, 1H), 2.10-2.01 (m, 1H), 1.97 (d, J = 15.6 Hz, 1H), 1.57- 1.49 (m, 2H), 1.47 (d, J = 6.4 Hz, 3H). |
| 12 | 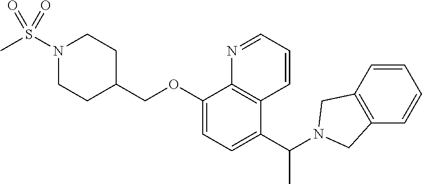 [SMILES: CC(c1ccc(OCC2CCN(S(C)(=O)=O)CC2)c2ncccc12)N1Cc2ccccc2C1] | MS m/z: 467.1 (M + 1)⁺. ¹H NMR (400 MHz, CD₃OD) δ 9.13 (dd, J = 1.2, 4.4 Hz, 1H), 9.00-8.95 (m, 1H), 8.43 (s, 1H), 7.78 (dd, J = 4.4, 8.4 Hz, 1H), 7.29 (s, 4H), 5.19-5.08 (m, 1H), 4.44-4.21 (m, 4H), 4.25 (d, J = 6.4 Hz, 2H), 3.88-3.86 (m, 2H), 3.88-3.86 (m, 2H), 2.83 (s, 3H), 2.32-2.27 (m, 1H), 2.22-2.20 (m, 2H), 1.74 (d, J = 6.4 Hz, 3H), 1.57-1.49 (m, 2H). |

### Example 13: 5-(isoindolin-2-ylmethyl)-2-((1R,5S,6r)-3-(methylsulfonyl)-3-azabicyclo[3.1.0]hexan-6-yl)-7H-furo[3,2-b]pyran-7-one

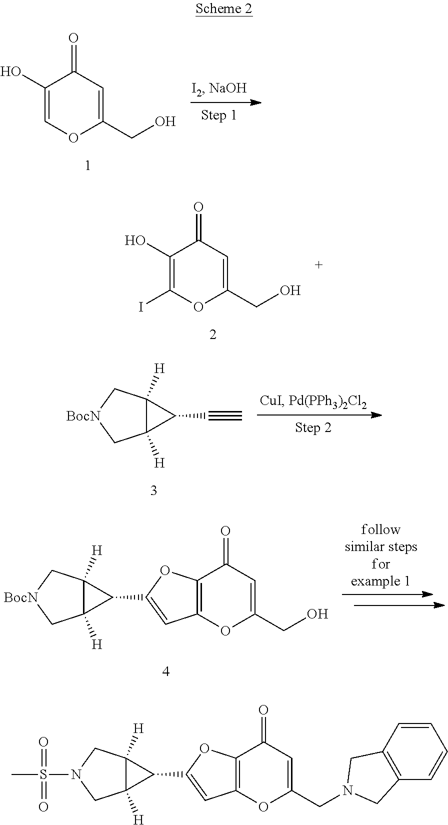

Step 1: To a solution of 5-hydroxy-2-(hydroxymethyl)pyran-4-one (100 g, 1.0 eq) in H₂O (1200 mL) was added NaOH (34 g, 1.2 eq), I₂ (214 g, 1.2 eq) was added in portions in ice bath. The reaction was stirred at 25° C. for 16 hr. The mixture was filtered and the cake was washed with EtOAc (30 ml×3). The crude product was triturated with EtOAc and concentrated under reduced pressure. Compound 2 (78 g, 42% yield) was obtained as a yellow solid.

MS m/z 269.0 (M+1)⁺.

Step 2: To a solution of 3 (8.7 g, 1.0 eq) (Bioorg. & Med. Chem., 2021, 30, 115964), 2 (11.25 g, 41.97 mmol, 1 eq), triethylamine (116.8 mL, 20.0 eq) in toluene (200 mL) was added cuprous iodide (1.60 g, 0.2 eq) and Pd(PPh₃)₂Cl₂ (2.95 g, 0.1 eq). The mixture was stirred at 30° C. for 16 h. The reaction was purified by flash chromatography to give 4 (5 g, 34% yield) as yellow solid. MS m/z: 348.2 (M+1)⁺.

¹H NMR (500 MHz, CDCl₃) δ 6.48 (s, 1H), 6.30 (s, 1H), 4.56 (s, 2H), 3.77-3.65 (m, 2H), 3.55-3.42 (m, 2H), 2.18 (t, J=2.5 Hz, 2H), 1.85 (t, J=3.5 Hz, 1H), 1.45 (s, 9H).

Following similar steps for example 1, 5-(isoindolin-2-ylmethyl)-2-((1R,5S,6r)-3-(methylsulfonyl)-3-azabicyclo[3.1.0]hexan-6-yl)-7H-furo[3,2-b]pyran-7-one could be obtained. MS m/z: 427.1 (M+1)⁺.

¹H NMR (400 MHz, CD₃OD) δ 7.43 (s, 4H), 6.75 (s, 1H), 6.73 (s, 1H), 4.83 (s, 4H), 4.74 (s, 2H), 3.68 (d, J=9.6 Hz, 2H), 3.51 (d, J=8.8 Hz, 2H), 2.94 (s, 3H), 2.33 (s, 3H).

Examples 14-54 were synthesized using similar methods as that of Example 13.

| Example No. | Structure | Mass and ¹HNMR |
|---|---|---|
| 14 | 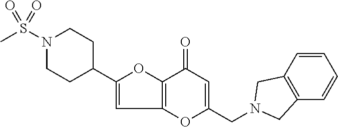 [SMILES: CS(=O)(=O)N1CCC(c2cc3oc(CN4Cc5ccccc5C4)cc(=O)c3o2)CC1] | MS m/z: 429.1 (M + 1)⁺. ¹H NMR (400 MHz, CDCl₃) δ 7.22 (s, 4H), 6.50 (s, 1H), 6.37 (s, 1H), 4.10 (s, 4H), 3.96-3.80 (m, 4H), 2.99-2.75 (m, 6H), 2.27-2.16 (m, 2H), 1.94-1.79 (m, 2H). |
| 15 | 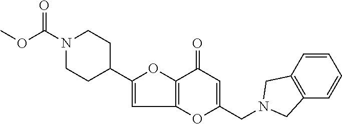 [SMILES: COC(=O)N1CCC(c2cc3oc(CN4Cc5ccccc5C4)cc(=O)c3o2)CC1] | MS m/z: 409.2 (M + 1)⁺. ¹H NMR (500 MHz, CD₃OD) δ 7.42 (s, 4H), 6.77 (s, 1H), 6.76 (s, 1H), 4.88-4.85 (m, 4H), 4.78 (s, 2H), 4.19 (d, J = 13.5 Hz, 2H), 3.70 (s, 3H), 3.19-3.10 (m, 1H), 3.10-2.95 (m, 2H), 2.16-2.04 (m, 2H), 1.75- 1.60 (m, 2H). |
| 16 | 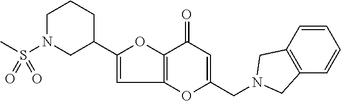 [SMILES: CS(=O)(=O)N1CCCC(c2cc3oc(CN4Cc5ccccc5C4)cc(=O)c3o2)C1] | MS m/z: 429.2 (M + 1)⁺. ¹H NMR (400 MHz, CD₃OD) δ 7.41 (s, 4H), 6.87 (s, 1H), 6.74 (s, 1H), 4.82 (br s, 4H), 4.72 (br s, 2H), 3.74 (d, J = 10.0 Hz, 1H), 3.43 (d, J = 4.0 Hz, 1H), 3.26 (d, J = 8.0 Hz, 2H), 3.03- 3.18 (m, 1H), 2.88 (s, 3H), 2.10 (br s, 1H), 1.93 (d, J = 8.8 Hz, 1H), 1.90-1.68 (m, 2H). |
| 17 | 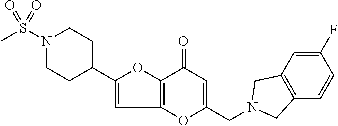 [SMILES: CS(=O)(=O)N1CCC(c2cc3oc(CN4Cc5ccc(F)cc5C4)cc(=O)c3o2)CC1] | MS m/z: 447.2 (M + 1)⁺. ¹H NMR (500 MHz, CDCl₃) δ 7.31 (s, 1H), 7.08-7.06 (m, 1H), 7.05-6.99 (m, 1H), 6.57 (s, 1H), 6.38 (s, 1H), 4.64 (s, 4H), 4.34 (s, 2H), 3.87-3.82 (m, 2H), 2.97-2.90 (m, 1H), 2.86-2.84 (m, 2H), 2.82 (s, 3H), 2.20-1.96 (m, 2H), 1.94-1.81 (m, 2H). |
| 18 | 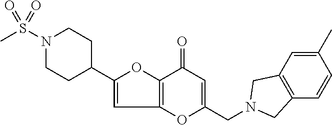 | MS m/z: 443.2 (M + H)⁺. ¹H NMR (400 MHz, CD₃OD) δ 7.33-7.26 (m, 1H), 7.24 (br s, 2H), 6.75 (d, J = 10.4 Hz, 2H), 4.78 (s, 4H), 4.73 (s, 2H), 3.82 (d, J = 12.0 Hz, 2H), 3.11-3.06 (m, 1H), 2.96-2.90 (m, 2H), 2.87 (s, 3H), 2.37 (s, 3H), 2.22-2.20 (m, 2H), 1.85-1.81 (m, 2H). |
| 19 | 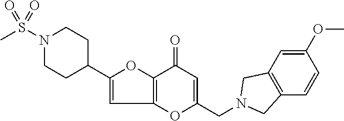 | MS m/z: 459.0 (M + 1)⁺. ¹H NMR (400 MHz, CD₃OD) δ 7.33-7.31 (m, 1H), 6.99-6.97 (m, 2 H), 6.79-6.75 (m, 2H), 4.77 (s, 2 H), 4.72 (s, 4 H), 3.86-3.83 (m, 1H), 3.83 (s, 3H), 3.11-3.09 (m, 2 H), 2.99- 2.98 (m, 2H), 2.95 (s, 3H), 2.24-2.21 (m, 2H), 1.87-1.83 (m, 2H). |
| 20 | 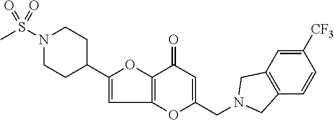 [SMILES: CS(=O)(=O)N1CCC(c2cc3oc(CN4Cc5ccc(C(F)(F)F)cc5C4)cc(=O)c3o2)CC1] | MS m/z: 497.0 (M + 1)⁺. ¹H NMR (400 MHz, CD₃OD) δ 7.74-7.70 (m, 2H), 7.60-7.58 (m, 1 H), 6.77 (s, 1H), 6.72 (s, 1H), 4.73 (s, 4H), 4.58 (s, 2H), 3.85-3.82 (m, 2H), 2.98-2.97 (m, 1H), 2.95-2.92 (m, 2H), 2.88 (s, 3H), 2.24-2.22 (m, 2H), 1.87-1.81 (m, 2H). |
| 21 | 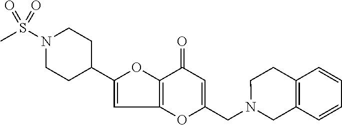 [SMILES: CS(=O)(=O)N1CCC(c2cc3oc(CN4CCc5ccccc5C4)cc(=O)c3o2)CC1] | MS m/z: 443.0 (M + 1)⁺. ¹H NMR (400 MHz, CD₃OD) δ 7.35- 7.30 (m, 3H), 7.23-7.21 (m, 1 H), 6.79-6.78 (m, 2H), 4.60-4.54 (m, 4 H), 3.85-3.82 (m, 2 H), 3.72-3.70 (m, 2H), 3.27-3.26 (m, 2H), 3.24-3.20 (m, 1 H), 2.98-2.97 (m, 2H), 2.88 (s, 3H), 2.24-2.22 (m, 2H), 1.89-1.83 (m, 2H). |
| 22 | 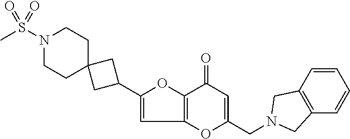 [SMILES: CS(=O)(=O)N1CCC2(CC1)CC(c1cc3oc(CN4Cc5ccccc5C4)cc(=O)c3o1)C2] | MS m/z: 469.1 (M + 1)⁺. ¹H NMR (400 MHz, CD₃OD) δ 7.42-7.39 (m, 4H), 6.76-6.73 (m, 2H), 4.85 (s, 4H), 4.77 (s, 2H), 3.81-3.76 (m, 1H), 3.24- 3.21 (m, 2H), 3.17-3.12 (m, 2H), 2.82 (s, 3H), 2.41-2.32 (m, 2H), 2.21-2.14 (m, 2H), 1.90-1.85 (m, 2H), 1.79-1.73 (m, 2H). |
| 23 | 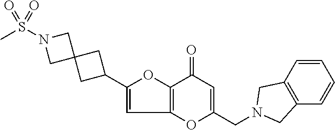 [SMILES: CS(=O)(=O)N1CC2(CC(c3cc4oc(CN5Cc6ccccc6C5)cc(=O)c4o3)C2)C1] | MS m/z: 441.1 (M + 1)⁺. ¹H NMR (400 MHz, CD₃OD) δ 7.23 (s, 4H), 6.70 (s, 1H), 6.55 (s, 1H), 4.07 (s, 4H), 4.04 (s, 2H), 3.97 (s, 2H), 3.93 (s, 2H), 3.70-3.61 (m, 1H), 2.75-2.65 (m, 2H), 2.62- 2.51 (m, 2H). |
| 24 | 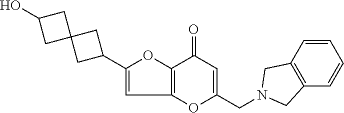 [SMILES: O=c1cc(CN2Cc3ccccc3C2)oc2cc(C3CC4(CC(O)C4)C3)oc12] | MS m/z: 378.0 (M + 1)⁺. ¹H NMR (400 MHz, CD₃OD) δ 7.44 (s, 4H), 6.77 (s, 1H), 6.73 (s, 1H), 4.87 (br s, 4H), 4.78 (s, 2H), 4.14 (t, J = 7.3 Hz, 1H), 3.66 (t, J = 8.5 Hz, 1H), 2.62-2.55 (m, 1H), 2.53- 2.46 (m, 1H), 2.43-2.32 (m, 4H), 2.03 (dd, J = 7.7, 11.0 Hz, 1H), 1.95 (dd, J = 7.7, 11.3 Hz, 1H). |
| 25 | 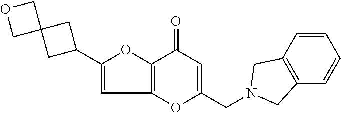 [SMILES: O=c1cc(CN2Cc3ccccc3C2)oc2cc(C3CC4(COC4)C3)oc12] | MS m/z: 364.2 (M + 1)⁺. ¹H NMR (400 MHz, CD₃OD) δ 7.27-7.18 (m, 4H), 6.66 (s, 1H), 6.54 (s, 1H), 4.80 (s, 2H), 4.68 (s, 2H), 4.06 (s, 4H), 3.95 (s, 2H), 3.54 (quin, J = 8.3 Hz, 1H), 2.77-2.68 (m, 2H), 2.58-2.50 (m, 2H). |
| 26 | 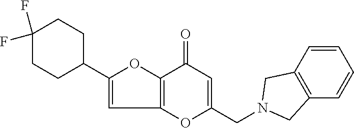 [SMILES: O=c1cc(CN2Cc3ccccc3C2)oc2cc(C3CCC(F)(F)CC3)oc12] | MS m/z: 386.0 (M + 1)⁺. ¹H NMR (400 MHz, CD₃OD) δ 7.43 (s, 4H), 6.78 (s, 1H), 6.76 (s, 1H), 4.90-4.81 (m, 4H), 4.77 (s, 2H), 3.22-3.01 (m, 1H), 2.60-1.76 (m, 8H). |
| 27 | 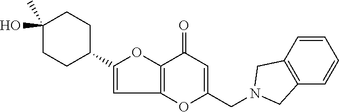 [SMILES: C[C@]1(O)CC[C@H](c2cc3oc(CN4Cc5ccccc5C4)cc(=O)c3o2)CC1] | MS m/z: 380.2 (M + 1)⁺. ¹H NMR (400 MHz, CD₃OD) δ 7.25-7.20 (m, 4H), 6.67 (s, 1H), 6.54 (s, 1H), 4.41 (s, 4H), 3.96 (s, 2H), 2.91-2.80 (m, 1H), 2.12-2.10 (m, 2H), 1.76-1.72 (m, 4H), 1.64-1.61 (m, 2H), 1.25 (s, 3H). |
| 28 | 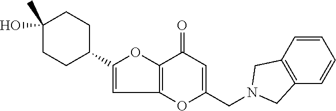 [SMILES: C[C@]1(O)CC[C@@H](c2cc3oc(CN4Cc5ccccc5C4)cc(=O)c3o2)CC1] | MS m/z: 380.2 (M + 1)⁺. ¹H NMR (400 MHz, CD₃OD) δ 7.25-7.20 (m, 4H), 6.61 (s, 1H), 6.53 (s, 1H), 4.06 (s, 4H), 3.96 (s, 2H), 2.78-2.77 (m, 1H), 1.92-1.88 (m, 4H), 1.79-1.76 (m, 2H), 1.58-1.54 (m, 2H), 1.24 (s, 3H). |
| 29 | 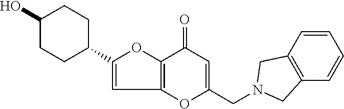 [SMILES: O=c1cc(CN2Cc3ccccc3C2)oc2cc([C@H]3CC[C@H](O)CC3)oc12] | MS m/z: 366.2 (M + 1)⁺. ¹H NMR (400 MHz, CD₃OD) δ 7.42 (s, 4H), 6.74 (s, 1H), 6.69 (s, 1H), 4.83 (s, 4H), 4.75 (s, 2H), 3.66-3.54 (m, 1H), 2.89- 2.78 (m, 1H), 2.20-2.12 (m, 2H), 2.11-2.04 (m, 2H), 1.68-1.54 (m, 2H), 1.49-1.36 (m, 2H) |
| 30 | 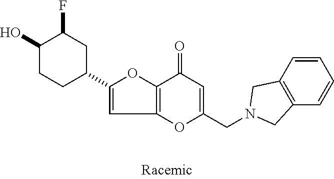 [SMILES: O=c1cc(CN2Cc3ccccc3C2)oc2cc([C@@H]3CC[C@@H](O)[C@@H](F)C3)oc12] | MS m/z: 384.0 (M + 1)⁺. ¹H NMR (500 MHz, CD₃OD) δ 7.27-7.17 (m, 4H), 6.67 (d, J = 0.6 Hz, 1H), 6.55-6.53 (m, 1H), 4.06 (s, 4H), 3.98-3.95 (m, 2H), 3.74-3.61 (m, 1H), 3.19-3.12 (m, 1H), 2.47-2.38 (m, 1H), 2.20-2.11 (m, 1H), 1.94-1.80 (m, 3H), 1.72-1.61 (m, 1H) |
| 31 | 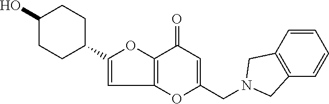 [SMILES: O=c1cc(CN2Cc3ccccc3C2)oc2cc([C@H]3CC[C@H](O)CC3)oc12] | MS m/z: 380.2 (M + 1)⁺. ¹H NMR (400 MHz, CD₃OD) δ 7.41 (s, 4H), 6.73 (s, 1H), 6.69 (s, 1H), 5.03-4.89 (m, 4H), 4.73 (d, J = 13.1 Hz, 3H), 3.66- 3.56 (m, 1H), 3.61 (tt, J = 4.2, 10.7 Hz, 1H), 2.83 (tt, J = 3.5, 11.9 Hz, 1H), 2.20-2.12 (m, 2H), 2.12- 2.04 (m, 2H), 1.87 (d, J = 6.8 Hz, 3H), 1.67-1.54 (m, 2H), 1.51-1.36 (m, 2H). |
| 32 | 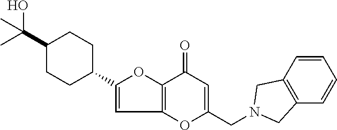 [SMILES: CC(C)(O)[C@H]1CC[C@H](c2cc3oc(CN4Cc5ccccc5C4)cc(=O)c3o2)CC1] | MS m/z: 408.2 (M + 1)⁺. ¹H NMR (400 MHz, CD₃OD) δ 7.27-7.199 (m, 4H), 6.62-6.60 (m, 1H), 6.54-6.52 (m, 1H), 4.11-4.03 (m, 4H), 3.99-3.94 (m, 2H), 2.83-2.72 (m, 1H), 2.26-2.17 (m, 2H), 2.06- 1.97 (m, 2H), 1.57-1.44 (m, 2H), 1.43-1.34 (m, 1H), 1.32-1.21 (m, 2H), 1.19-1.16 (m, 6H). |
| 33 | 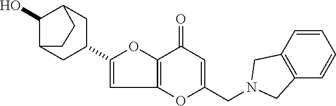 [SMILES: O=c1cc(CN2Cc3ccccc3C2)oc2cc([C@H]3CC4CCC(C3)[C@H]4O)oc12] | MS m/z: 392.0 (M + 1)⁺. ¹H NMR (500 MHz, CD₃OD) δ 7.43 (s, 4H), 6.74 (s, 1H), 6.69 (s, 1H), 4.86- 4.84 (m, 4H), 4.76 (s, 2H), 3.97 (t, J = 4.8 Hz, 1H), 3.29- 3.22 (m, 1H), 2.22-2.14 (m, 4H), 1.90-1.84 (m, 2H), 1.70-1.65 (m, 4H). |
| 34 | 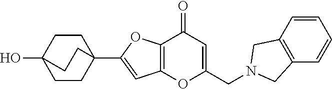 [SMILES: O=c1cc(CN2Cc3ccccc3C2)oc2cc(C34CCC(O)(CC3)CC4)oc12] | MS m/z: 392.0 (M + 1)⁺. ¹H NMR (400 MHz, CD₃OD) δ 7.42 (s, 4H), 6.76 (s, 1H), 6.66 (s, 1H), 4.92-4.81 (m, 4H), 4.78 (s, 2H), 2.10-2.03 (m, 6H), 1.81-1.73 (m, 6H). |
| 35 | 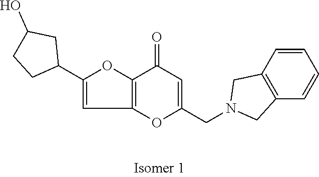 [SMILES: O=c1cc(CN2Cc3ccccc3C2)oc2cc(C3CCC(O)C3)oc12] | MS m/z: 352.2 (M + 1)⁺. ¹H NMR (400 MHz, CD₃OD) δ 7.42 (s, 4H), 6.81-6.66 (m, 2H), 4.88-4.82 (m, 4H), 4.77 (s, 2H), 4.53-4.27 (m, 1H), 3.60 (t, J = 8.8 Hz, 1H), 2.50-2.38 (m, 1H), 2.35-2.23 (m, 1H), 2.18-2.06 (m, 2H), 2.05-1.95 (m, 1H), 1.88-1.70 (m, 2H). |
| 36 | 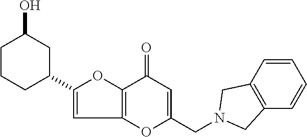 [SMILES: O=c1cc(CN2Cc3ccccc3C2)oc2cc([C@@H]3CCC[C@@H](O)C3)oc12] | MS m/z: 366.2 (M + 1)⁺. ¹H NMR (400 MHz, CD₃OD) δ 7.28-7.20 (m, 4H), 6.63 (d, J = 0.7 Hz, 1H), 6.54 (s, 1H), 4.60 (br s, 2H), 4.08 (s, 4H), 3.97 (s, 2H), 2.12-1.95 (m, 3H), 1.88-1.81 (m, 2H), 1.76-1.71 (m, 1H), 1.68-1.55 (m, 4H). |
| 37 | 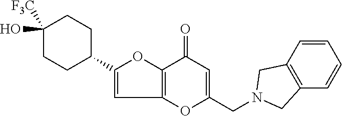 [SMILES: O=c1cc(CN2Cc3ccccc3C2)oc2cc([C@H]3CC[C@@](O)(C(F)(F)F)CC3)oc12] | MS m/z: 434.1 (M + 1)⁺. ¹H NMR (400 MHz, CD₃OD) δ 7.29-7.18 (m, 4H), 6.82 (d, J = 1.3 Hz, 1H), 6.56 (s, 1H), 4.08 (s, 4H), 3.98 (s, 2H), 3.27-3.22 (m, 1H), 2.23-2.13 (m, 4H), 1.80-1.70 (m, 4H). |
| 38 | 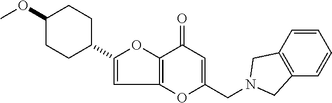 [SMILES: CO[C@H]1CC[C@H](c2cc3oc(CN4Cc5ccccc5C4)cc(=O)c3o2)CC1] | MS m/z: 380.1 (M + 1)⁺. ¹H NMR (400 MHz, CD₃OD) δ 7.42 (s, 4H), 6.75 (s, 1H), 6.70 (s, 1H), 4.84 (br s, 4H), 4.76 (s, 2H), 3.38 (s, 3H), 3.27 (t, J = 3.9 Hz, 1H), 2.91-2.80 (m, 1H), 2.25-2.15 (m, 4H), 1.67-1.55 (m, 2H), 1.41-1.33 (m, 2H). |
| 39 | 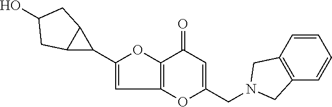 [SMILES: O=c1cc(CN2Cc3ccccc3C2)oc2cc(C3C4CC(O)CC43)oc12] | MS m/z: 364.2 (M + 1)⁺. ¹H NMR (400 MHz, CD₃OD) δ 7.42 (s, 4H), 6.71 (s, 1H), 6.57 (s, 1H), 4.84 (br s, 4H), 4.74 (s, 2H), 4.10 (quin, J = 7.6 Hz, 1H), 2.30 (dd, J = 7.2, 13.1 Hz, 2H), 1.98 (dd, J = 3.0, 6.6 Hz, 3H), 1.89-1.79 (m, 2H) |
| 40 | 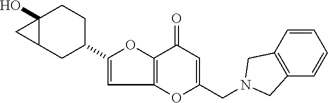 [SMILES: O=c1cc(CN2Cc3ccccc3C2)oc2cc([C@H]3CC[C@@]4(O)CC4C3)oc12] | MS m/z: 378.2 (M + 1)⁺. ¹H NMR (400 MHz, CD₃Cl) δ 7.25-7.14 (m, 4H), 6.52-6.42 (m, 1H), 6.40-6.19 (m, 1H), 4.16-4.03 (m, 4H), 3.95-3.79 (m, 2H), 2.87-2.60 (m, 1H), 2.58-2.36 (m, 1H), 2.22-1.72 (m, 4H), 1.56-1.43 (m, 1H), 1.40-1.15 (m, 2H), 1.07-0.93 (m, 1H), 0.57-0.31 (m, 1H). |
| 41 | 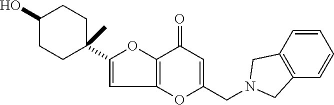 [SMILES: C[C@]1(c2cc3oc(CN4Cc5ccccc5C4)cc(=O)c3o2)CC[C@H](O)CC1] | MS m/z: 380.0 (M + 1)⁺. ¹H NMR (400 MHz, CD₃OD) δ 7.43 (s, 4H), 6.76 (d, J = 2.7 Hz, 2H), 4.86 (br s, 4H), 4.78 (s, 2H), 3.77-3.67 (m, 1H), 1.99-1.87 (m, 4H), 1.83-1.71 (m, 2H), 1.70-1.57 (m, 2H), 1.39 (s, 3H). |
| 42 | 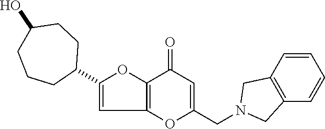 [SMILES: O=c1cc(CN2Cc3ccccc3C2)oc2cc([C@@H]3CCC[C@@H](O)CC3)oc12] | MS m/z: 380.2 (M + 1)⁺. ¹H NMR (400 MHz, CD₃OD) δ 7.44 (s, 4H), 6.77 (s, 1H), 6.72 (s, 1H), 4.87 (br s, 4H), 4.79 (s, 2H), 3.95-3.80 (m, 1H), 3.20- 3.10 (m, 1H), 2.18-2.07 (m, 3H), 2.02-1.93 (m, 1H), 1.87-1.76 (m, 2H), 1.76-1.65 (m, 4H). |
| | Racemic | |
| 43 | 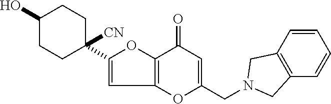 [SMILES: N#C[C@]1(c2cc3oc(CN4Cc5ccccc5C4)cc(=O)c3o2)CC[C@H](O)CC1] | MS m/z: 391.2 (M + 1)⁺. ¹H NMR (400 MHz, CD₃OD) δ 7.43 (s, 4H), 7.09 (s, 1H), 6.80 (s, 1H), 4.84-4.81 (m, 4H), 4.76 (s, 2H), 4.06 (br s, 1H), 2.47-2.35 (m, 2H), 2.16 (d, J = 13.5 Hz, 2H), 2.03-1.93 (m, 2H), 1.92-1.73 (m, 2H). |
| 44 | 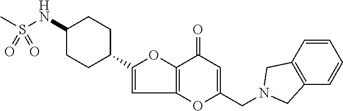 [SMILES: CS(=O)(=O)N[C@H]1CC[C@H](c2cc3oc(CN4Cc5ccccc5C4)cc(=O)c3o2)CC1] | MS m/z: 443.2 (M + 1)⁺. ¹H NMR (400 MHz, CDCl₃) δ 7.20 (s, 4H), 6.47 (s, 1H), 6.30 (s, 1H), 4.44- 4.31 (m, 1H), 4.18 (s, 4H), 3.90 (s, 2H), 3.41-3.29 (m, 1H), 3.00 (s, 3H), 2.72-2.67 (m, 1H), 2.29-2.16 (m, 4H), 1.64-1.53 (m, 2H), 1.46-1.38 (m, 2H). |
| 45 | 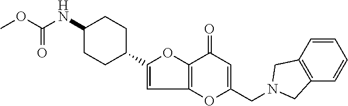 [SMILES: COC(=O)N[C@H]1CC[C@H](c2cc3oc(CN4Cc5ccccc5C4)cc(=O)c3o2)CC1] | MS m/z: 423.0 (M + 1)⁺. ¹H NMR (500 MHz, CD₃OD) δ 7.24-7.20 (m, 4H), 6.63 (s, 1H), 6.54 (s, 1H), 4.07 (s, 4H), 3.96 (s, 2H), 3.63 (s, 3H), 3.48-3.40 (m, 1H), 2.82-2.80 (m, 1H), 2.19-2.17 (m, 2H), 2.08-2.04 (m, 2H), 1.63-1.60 (m, 2H), 1.40-1.38 (m, 2H). |
| 46 | 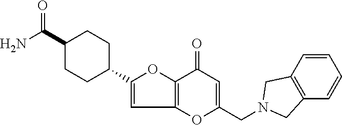 [SMILES: NC(=O)[C@H]1CC[C@H](c2cc3oc(CN4Cc5ccccc5C4)cc(=O)c3o2)CC1] | MS m/z: 393.2 (M + 1)⁺. ¹H NMR (400 MHz, CD₃OD) δ 7.45-7.40 (m, 4H), 6.78 (s, 1H), 6.74 (s, 1H), 4.97-4.82 (m, 4H), 4.80 (s, 2H), 2.91-2.90 (m, 1H), 2.42-2.40 (m, 1H), 2.23-2.21 (m, 2H), 2.06-2.03 (m, 2H), 1.68-1.59 (m, 4H). |
| 47 | 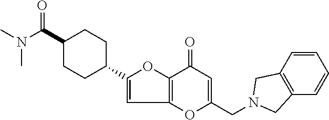 [SMILES: CN(C)C(=O)[C@H]1CC[C@H](c2cc3oc(CN4Cc5ccccc5C4)cc(=O)c3o2)CC1] | MS m/z: 421.3 (M + 1)⁺. ¹H NMR (400 MHz, CD₃OD) δ 7.42 (s, 4H), 6.73 (s, 1H), 6.68 (s, 1H), 4.83 (s, 4H), 4.74 (s, 2H), 3.14 (s, 3H), 2.94 (s, 3H), 2.90 (d, J = 3.2 Hz, 1H), 2.77 (br s, 1H), 2.14-2.23 (m, 2H), 1.92 (d, J = 7.6 Hz, 2H), 1.60-1.72 (m, 4H). |
| 48 | 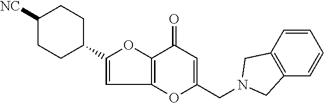 [SMILES: N#C[C@H]1CC[C@H](c2cc3oc(CN4Cc5ccccc5C4)cc(=O)c3o2)CC1] | MS m/z: 375.1 (M + 1)⁺. ¹H NMR (500 MHz, CD₃OD) δ 7.44 (s, 4H), 6.77 (s, 1H), 6.74 (s, 1H), 4.88 (br s, 4H), 4.79 (s, 2H), 3.04-2.91 (m, 1H), 2.81-2.64 (m, 1H), 2.26-2.20 (m, 4H), 1.84-1.72 (m, 2H), 1.68-1.62 (m, 2H) |
| 49 | 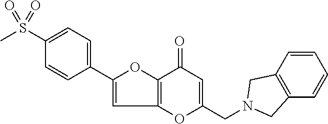 [SMILES: CS(=O)(=O)c1ccc(-c2cc3oc(CN4Cc5ccccc5C4)cc(=O)c3o2)cc1] | MS m/z: 422.0 (M + 1)⁺ ¹H NMR (500 MHz, CD₃OD) δ 8.25-8.20 (d, J = 8.5 Hz, 2 H), 8.15-8.10 (d, J = 8.5 Hz, 2H), 7.60 (s, 1 H), 7.43 (s, 4 H), 6.82 (s, 1 H), 4.81 (s, 4 H), 4.73 (s, 2 H), 3.19 (s, 3 H). |
| 50 | 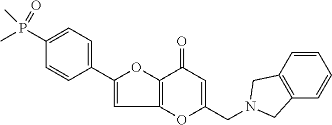 [SMILES: CP(C)(=O)c1ccc(-c2cc3oc(CN4Cc5ccccc5C4)cc(=O)c3o2)cc1] | MS m/z: 420.1 (M + 1)⁺ ¹H NMR (400 MHz, CD₃OD) δ 8.17-8.14 (m, 2H), 8.00-7.97 (m, 2H), 7.52 (s, 1H), 7.26-7.23 (m, 4H), 6.65 (s, 1H), 4.12 (s, 4H), 4.03 (s, 2H), 1.87 (s, 3H), 1.84 (s, 3H). |
| 51 | 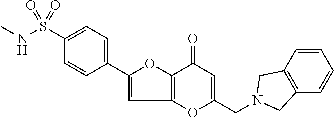 [SMILES: CNS(=O)(=O)c1ccc(-c2cc3oc(CN4Cc5ccccc5C4)cc(=O)c3o2)cc1] | MS m/z: 437.0 (M + 1)⁺. ¹H NMR (400 MHz, CD₃OD) δ 7.96-7.91 (d, J = 8.8 Hz 2H), 7.44-7.43 (m, 4H), 7.40-7.37 (d, J = 8.8 Hz 2H), 7.30 (s, 1H), 6.80 (s, 1H), 4.88 (s, 4H), 4.78 (s, 2H), 3.11 (s, 3H). |
| 52 | 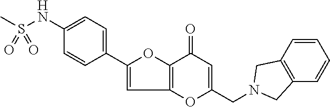 [SMILES: CS(=O)(=O)Nc1ccc(-c2cc3oc(CN4Cc5ccccc5C4)cc(=O)c3o2)cc1] | MS m/z: 437.0 (M + 1)⁺. ¹H NMR (400 MHz, CD₃OD) δ 7.96-7.91 (m, 2H), 7.43 (s, 4H), 7.41-7.37 (m, 2H), 7.30 (s, 1H), 6.80 (s, 1H), 4.88 (s, 4H), 4.78 (s, 2H), 3.19 (s, 3H). |
| 53 | 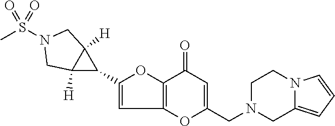 [SMILES: CS(=O)(=O)N1C[C@@H]2[C@H](C1)[C@H]2c1cc2oc(CN3CCn4cccc4C3)cc(=O)c2o1] | MS: 430.0 (M + 1)⁺. ¹H NMR (400 MHz, CD₃OD) δ 6.76 (d, J = 5.7 Hz, 3H), 6.19-6.15 (m, 1H), 6.07 (d, J = 3.3 Hz, 1H), 4.66 (s, 2H), 4.59 (br s, 2H), 4.35 (t, J = 5.8 Hz, 2H), 3.90 (br s, 2H), 3.65 (d, J = 9.7 Hz, 2H), 3.50 (br d, 9.5 Hz, 2H), 2.92 (s, 3H), 2.31 (s, 3H) |
| 54 | 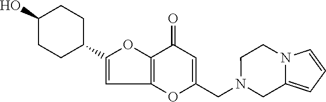 [SMILES: O=c1cc(CN2CCn3cccc3C2)oc2cc([C@H]3CC[C@H](O)CC3)oc12] | MS: 369.1 (M + 1)⁺. ¹H NMR (400 MHz, CD₃OD) δ 6.82 (s, 1H), 6.77 (s, 1H), 6.71 (s, 1H), 6.22-6.19 (m, 1H), 6.10 (br d, J = 3.0 Hz, 1H), 4.66 (s, 2H), 4.60 (s, 2H), 4.36 (br t, J = 5.7 Hz, 2H), 3.92-3.87 (m, 2H), 3.62 (s, 1H), 2.88-2.80 (m, 1H), 2.21-2.15 (m, 2H), 2.09 (br dd, J = 2.6, 12.3 Hz, 2H), 1.66-1.58 (m, 2H), 1.46 (br s, 2H) |

### Example 55: 6-(isoindolin-2-ylmethyl)-2-(1-(methylsulfonyl)piperidin-4-yl)quinoxaline

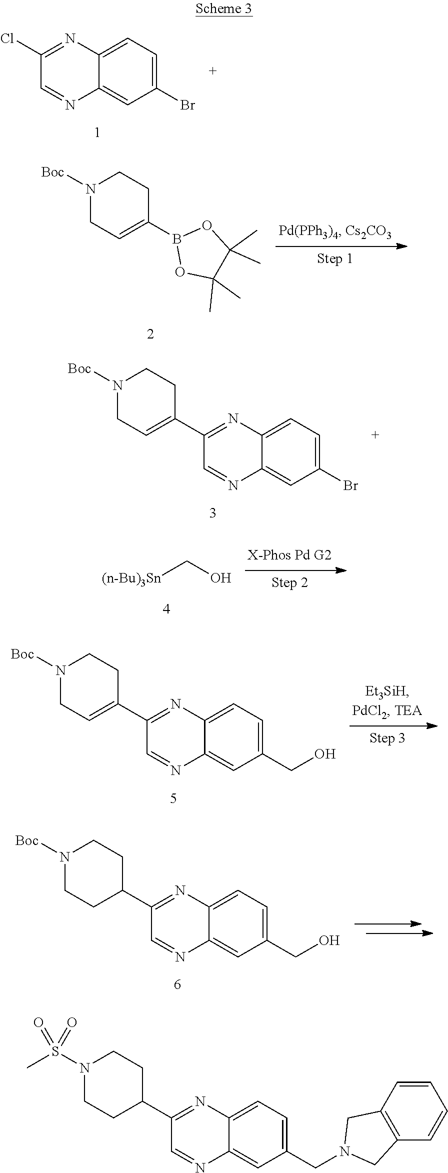

Step 1: To a solution of 1 (5.0 g, 1.0 eq) in THE (100 mL) and H₂O (20 mL) was added 2 (7.6 g, 1.2 eq), Cs₂CO₃ (20.1 g, 3.0 eq) and Pd(PPh₃)₄ (2.37 g, 0.1 eq). The mixture was stirred at 80° C. for 1 h under N₂ atmosphere. The reaction was purified by flash chromatography to give 3 (7.7 g, 96% yield) as a red solid.

MS m/z: 392.1 (M+1)⁺.

¹H NMR (400 MHz, CDCl₃) δ 9.05 (s, 1H), 8.23 (d, J=2.0 Hz, 1H), 7.95-7.86 (m, 1H), 7.82-7.76 (m, 1H), 6.82 (br s, 1H), 4.23 (d, J=1.6 Hz, 2H), 3.70 (t, J=5.6 Hz, 2H), 2.83 (br s, 2H), 1.51 (s, 9H).

Step 2: To a solution of 3 (7.7 g, 1.0 eq) in toluene (100 mL) was added tributylstannylmethanol (4, 9.5 g, 1.5 eq) and X-Phos Pd G2 (1.6 g, 0.1 eq). The mixture was stirred at 90° C. for 1 h. The reaction was purified by flash chromatography to give 5 (4.0 g, 59% yield) as a red solid.

MS m/z: 342.0 (M+1)⁺.

Step 3: To a solution of 5 (1.9 g, 1.0 eq) in THE (20 mL) was added TEA (1.5 mL, 3.0 eq), Et₃SiH (1.7 mL, 3.0 eq) and PdCl₂ (63 mg, 0.1 eq). The mixture was stirred at 25° C. for 1 h. The reaction was purified by flash chromatography to give 6 (1.9 g, 100% yield) as a white solid.

MS m/z: 344.1 (M+1)⁺.

Following similar steps for example 1, Example 55 compound could be obtained.

MS m/z: 423.0 (M+H)⁺.

¹H NMR (400 MHz, CD₃OD) δ 8.98 (s, 1H), 8.33 (br s, 1H), 8.22 (d, J=8.4 Hz, 1H), 8.01 (d, J=8.4 Hz, 1H), 7.41 (s, 4H), 4.83-4.73 (m, 6H), 3.92 (d, J=11.6 Hz, 2H), 3.28-3.19 (m, 1H), 3.04-2.96 (m, 2H), 2.91 (s, 3H), 2.22-2.14 (m, 2H), 2.11-1.93 (m, 2H).

### Example 56: 6-(isoindolin-2-ylmethyl)-2-((1R,5S,6r)-3-(methylsulfonyl)-3-azabicyclo[3.1.0]hexan-6-yl)quinoline

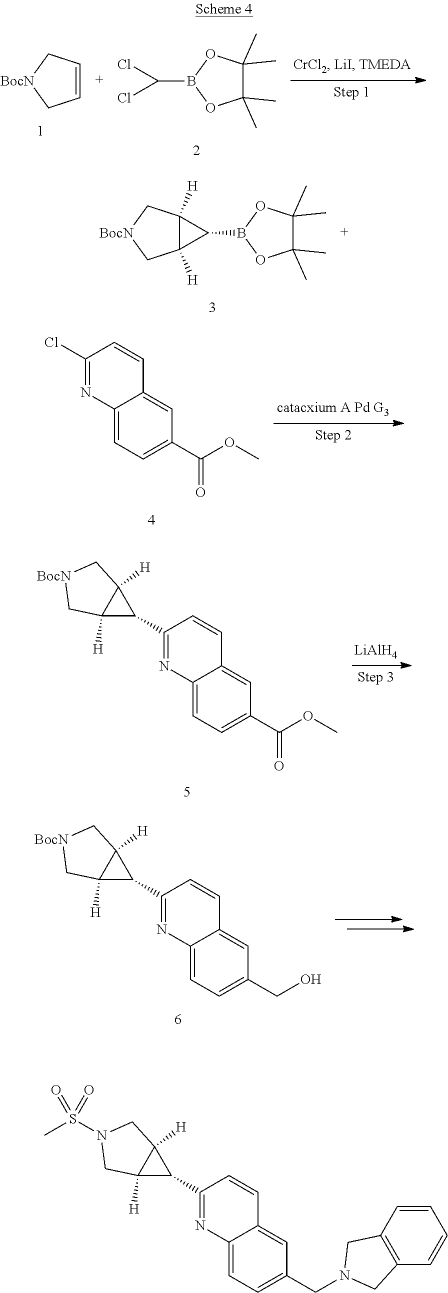

Step 1: TMEDA (3.6 mL, 8.0 eq) was added to a solution of chromium dichloride (2.9 g, 8.0 eq) in THE (60 mL). The resultant blue mixture was stirred at 20° C. for 15 mins 1 (500 mg, 1.0 eq) in THE (20 mL) was added followed by a solution of 2 (1.25 g, 2.0 eq) and Lil (1.6 g, 4.0 eq) in THF (30 mL). The mixture was stirred at 20° C. for 16 hours. The reaction was purified by flash chromatography to give 3 (300 mg, 33% yield) as colorless oil.

¹H NMR (400 MHz, CDCl₃) δ 3.64-3.49 (m, 2H), 3.39-3.25 (m, 2H), 1.68-1.60 (m, 2H), 1.45-1.39 (m, 9H), 1.22 (s, 12H), 0.15-0.31 (m, 1H).

Step 2: To a solution of 3 (260 mg, 1.0 eq) and 4 (223 mg, 1.2 eq) in H₂O (2 mL) and 2-methylbutan-2-ol (8 mL) was added cataCXium A Pd G3 (61, 0.1 eq) and Cs₂CO₃ (821 mg, 3.0 eq). The mixture was stirred at 90° C. for 1 h. The reaction was purified by flash chromatography to give 5 (140 mg, 45% yield) as yellow solid.

MS m/z: 369.2 (M+1)⁺.

¹H NMR (400 MHz, CDCl₃) δ 8.53 (d, J=1.2 Hz, 1H), 8.29-8.21 (m, 1H), 8.17-8.07 (m, 1H), 8.04-7.89 (m, 1H), 7.31 (d, J=8.4 Hz, 1H), 3.99 (s, 3H), 3.84 (d, J=11.2 Hz, 1H), 3.78-3.73 (m, 1H), 3.61-3.51 (m, 2H), 2.39-2.26 (m, 2H), 2.12-2.03 (m, 1H), 1.49 (s, 9H).

Step 3: To a solution of 5 (140 mg, 1.0 eq) in THE (6 mL) was added LiAlH₄ (43 mg, 3.0 eq) at −78° C. The mixture was stirred at 0° C. for 10 min. The reaction was purified by flash chromatography to give the crude 6 (110 mg, 85% yield) as white solid.

MS m/z: 341.2 (M+1)⁺.

¹H NMR (400 MHz, CDCl₃) δ 8.09-7.89 (m, 2H), 7.75 (s, 1H), 7.66 (d, J=8.4 Hz, 1H), 7.22 (d, J=8.4 Hz, 1H), 4.91-4.83 (m, 2H), 3.86-3.72 (m, 2H), 3.60-3.50 (m, 2H), 2.34-2.23 (m, 2H), 1.93-1.83 (m, 1H), 1.49 (s, 9H).

Following similar steps for Example 1, 6-(isoindolin-2-ylmethyl)-2-((1R,5S,6r)-3-(methylsulfonyl)-3-azabicyclo[3.1.0]hexan-6-yl)quinoline could be obtained.

MS m/z: 420.1 (M+1)⁺.

¹H NMR (500 MHz, CD₃OD) δ 9.00 (d, J=8.5 Hz, 1H), 8.53 (s, 1H), 8.34-8.27 (m, 2H), 7.75 (d, J=8.5 Hz, 1H), 7.47-7.40 (m, 4H), 4.96 (s, 2H), 4.82 (s, 4H), 3.85 (d, J=10.0 Hz, 2H), 3.67 (d, J=10.0 Hz, 2H), 3.02 (s, 3H), 2.75-2.70 (m, 3H).

Examples 57-133 were synthesized using similar methods utilized in Examples 55 and 56.

| Example | | |
|---|---|---|
| No. | Structure | Mass and ¹HNMR |
| 57 | 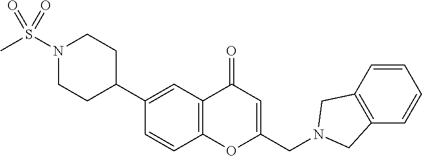 [SMILES: CS(=O)(=O)N1CCC(c2ccc3oc(CN4Cc5ccccc5C4)cc(=O)c3c2)CC1] | MS m/z: 439.2 (M + 1)⁺. ¹H NMR (400 MHz, CD₃OD) δ 8.05 (d, J = 2.0 Hz, 1H), 7.83 (dd, J = 2.4, 8.8 Hz, 1H), 7.74-7.62 (m, 1H), 7.44 (s, 4H), 6.67-6.65 (m, 1H), 4.93 (s, 4H), 4.78 (s, 2H), 3.89-3.87 (m, 2H), 2.96-2.93 (m, 3H), 2.89 (s, 3H), 2.07-1.97 (m, 2H), 1.89-1.82 (m, 2H). |
| 58 | 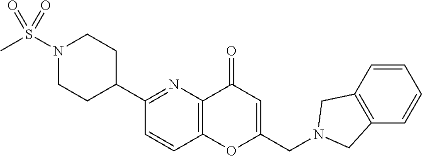 [SMILES: CS(=O)(=O)N1CCC(c2ccc3oc(CN4Cc5ccccc5C4)cc(=O)c3n2)CC1] | MS m/z: 440.2 (M + 1)⁺. ¹H NMR (400 MHz, CD₃OD) δ 8.18 (d, J = 8.8 Hz, 1H), 7.89 (d, J = 8.8 Hz, 1H), 7.43 (s, 4H), 6.86 (s, 1H), 4.98- 4.93 (m, 4H), 4.82 (s, 2H), 3.90-3.87 (m, 2H), 3.16-3.09 (m, 1H), 2.96-2.93 (m, 2H), 2.89 (s, 3H), 2.12-2.05 (m, 2H), 2.04-1.94 (m, 2H). |
| 59 | 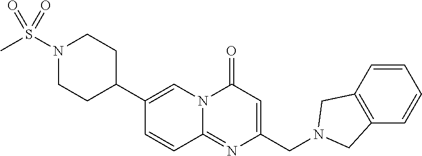 [SMILES: CS(=O)(=O)N1CCC(c2ccc3nc(CN4Cc5ccccc5C4)cc(=O)n3c2)CC1] | MS m/z: 439.1 (M + 1)⁺. ¹H NMR (400 MHz, CD₃OD) δ 9.00 (d, J = 1.2 Hz, 1H), 8.11 (dd, J = 1.6, 9.2 Hz, 1H), 7.84 (d, J = 9.2 Hz, 1H), 7.43 (s, 4H), 6.53 (s, 1H), 4.85 (s, 4H), 4.74 (s, 2H), 3.91 (d, J = 12.0 Hz, 2H), 2.97-2.96 (m, 1H), 2.94-2.93 (m, 2H), 2.90 (s, 3H), 2.10- 2.08 (m, 2H), 1.91-1.87 (m, 2H). |
| 60 | 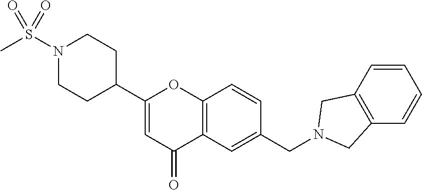 [SMILES: CS(=O)(=O)N1CCC(c2cc(=O)c3cc(CN4Cc5ccccc5C4)ccc3o2)CC1] | MS m/z: 439.0 (M + 1)⁺. ¹H NMR (400 MHz, CD₃OD) δ 8.37 (d, J = 2.0 Hz, 1H), 8.02 (dd, J = 2.0, 8.8 Hz, 1H), 7.79 (d, J = 8.4 Hz, 1H), 7.43 (s, 4H), 6.39 (s, 1H), 4.81 (s, 2H), 4.75 (s, 4H), 3.92-3.89 (m, 2H), 2.97-2.89 (m, 3H), 2.88 (s, 3H), 2.20-2.17 (m, 2H), 1.98-1.91 (m, 2H). |
| 61 | 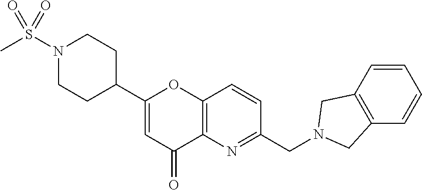 [SMILES: CS(=O)(=O)N1CCC(c2cc(=O)c3nc(CN4Cc5ccccc5C4)ccc3o2)CC1] | MS m/z: 440.2 (M + 1)⁺. ¹H NMR (400 MHz, CD₃OD) δ 8.12 (d, J = 8.8 Hz, 1H), 8.00 (d, J = 8.8 Hz, 1H), 7.21 (s, 4H), 6.50 (s, 1H), 4.22 (s, 2H), 4.03 (s, 4H), 3.89 (d, J = 11.6 Hz, 2H), 2.96-2.89 (m, 3H), 2.89 (s, 3H), 2.22-2.16 (m, 2H), 1.97-1.86 (m, 2H). |
| 62 | 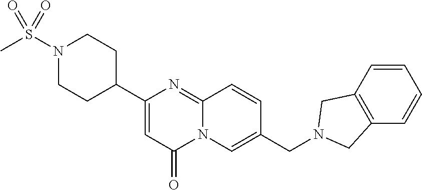 [SMILES: CS(=O)(=O)N1CCC(c2cc(=O)n3cc(CN4Cc5ccccc5C4)ccc3n2)CC1] | MS m/z: 439.1 (M + 1)⁺. ¹H NMR (400 MHz, CD₃OD) δ 9.49 (s, 1H), 8.56 (d, J = 7.2 Hz, 1H), 8.06 (d, J = 8.4 Hz, 1H), 7.42 (s, 4H), 6.61 (s, 1H), 4.93 (br s, 2H), 4.82 (br s, 4H), 3.92 (d, J = 11.2 Hz, 2H), 2.99-2.92 (m, 2H), 2.90 (s, 3H), 2.67 (br s, 1H), 2.18-2.13 (m, 2H), 1.97- 1.93 (m, 2H). |
| 63 | 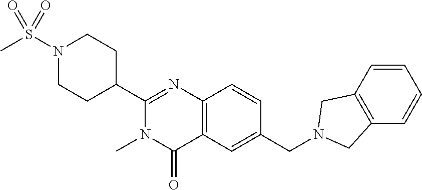 [SMILES: Cn1c(C2CCN(S(C)(=O)=O)CC2)nc2ccc(CN3Cc4ccccc4C3)cc2c1=O] | MS m/z: 453.1 (M + 1)⁺. ¹H NMR (400 MHz, CD₃OD) δ 8.42 (d, J = 1.6 Hz, 1H), 7.97 (dd, J = 2.0, 8.4 Hz, 1H), 7.82 (d, J = 8.4 Hz, 1H), 7.43-7.38 (m, 4H), 4.79 (s, 2H), 4.73 (s, 4H), 3.88 (d, J = 12.0 Hz, 2H), 3.75 (s, 3H), 3.27- 3.19 (m, 1H), 2.97 (d, J = 2.8 Hz, 2H), 2.90 (s, 3H), 2.15-2.03 (m, 4H). |
| 64 | 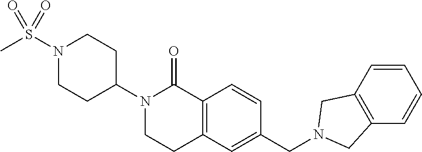 [SMILES: CS(=O)(=O)N1CCC(N2CCc3cc(CN4Cc5ccccc5C4)ccc3C2=O)CC1] | MS m/z: 440.1 (M + 1)⁺. ¹H NMR (500 MHz, CD₃OD) δ 8.10 (d, J = 8.0 Hz, 1H), 7.60-7.58 (m, 1H), 7.49-7.41 (m, 5H), 4.96-4.91 (m, 2H), 4.87- 4.75 (m, 4H), 4.69 (s, 2H), 4.52-4.44 (m, 1H), 3.62-3.60 (m, 2H), 3.09-3.07 (m, 2H), 2.96-2.92 (m, 2H), 2.90 (s, 3H), 2.05-1.97 (m, 2H), 1.87- 1.84 (m, 2H). |
| 65 | 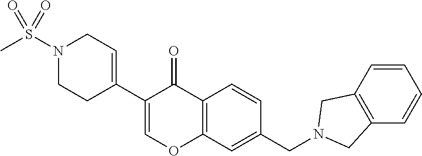 [SMILES: CS(=O)(=O)N1CC=C(c2coc3cc(CN4Cc5ccccc5C4)ccc3c2=O)CC1] | MS m/z: 437.1 (M + 1)⁺. ¹H NMR (500 MHz, DMSO-d6) δ 8.42 (s, 1H), 8.24-8.13 (m, 1H), 8.03-7.95 (m, 1H), 7.77-7.67 (m, 1H), 7.39 (br s, 4H), 6.43-6.32 (m, 1H), 4.80 (s, 2H), 4.66 (s, 4H), 3.87-3.82 (m, 2H), 3.38-3.35 (m, 2H), 2.95 (s, 3H), 2.59-2.54 (m, 2H). |
| 66 | 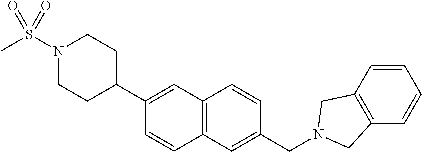 [SMILES: CS(=O)(=O)N1CCC(c2ccc3cc(CN4Cc5ccccc5C4)ccc3c2)CC1] | MS m/z: 421.0 (M + 1)⁺. ¹H NMR (400 MHz, DMSO-d6) δ 7.87 (d, J = 3.2 Hz, 1H), 7.85-7.83 (m, 2H), 7.76 (s, 1H), 7.53 (dd, J = 8.4, 1.6 Hz, 1H), 7.46 (dd, J = 8.4, 1.6 Hz, 1H), 7.20 (s, 4H), 4.02 (s, 2H), 3.87 (s, 4H), 3.77-3.69 (m, 2H), 2.93 (s, 3H), 2.91-2.78 (m, 3H), 2.03-1.93 (m, 2H), 1.87-1.74 (m, 2H). |
| 67 | 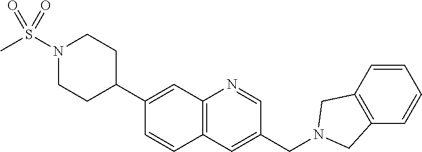 [SMILES: CS(=O)(=O)N1CCC(c2ccc3cc(CN4Cc5ccccc5C4)cnc3c2)CC1] | MS m/z: 422.1 (M + 1)⁺. ¹H NMR (400 MHz, CD₃OD) δ 9.18 (d, J = 8.8 Hz, 1H), 8.60 (d, J = 1.2 Hz, 1H), 8.47-8.43 (m, 1H), 8.40-8.36 (m, 1H), 8.20 (d, J = 8.8 Hz, 1H), 7.42 (s, 4H), 4.99 (s, 2H), 4.83 (s, 4H), 4.01 (m, 2H), 3.54-3.46 (m, 1H), 3.08-3.00 (m, 2H), 2.96 (s, 3H), 2.29-2.23 (m, 2H), 2.22-2.11 (m, 2H). |
| 68 | 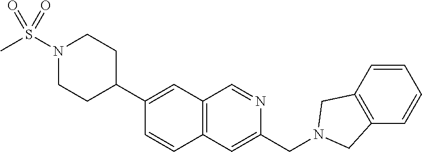 [SMILES: CS(=O)(=O)N1CCC(c2ccc3cc(CN4Cc5ccccc5C4)ncc3c2)CC1] | MS m/z: 422.0 (M + 1)⁺. ¹H NMR (400 MHz, DMSO-d6) δ 8.30 (d, J = 8.4 Hz, 1H), 7.94 (d, J = 8.8 Hz, 1H), 7.82 (d, J = 2.0 Hz, 1H), 7.71 (dd, J = 8.8, 2.0 Hz, 1H), 7.65 (d, J = 8.4 Hz, 1H), 7.20 (s, 4H), 4.16 (s, 2H), 3.94 (s, 4H), 3.77-3.69 (m, 2H), 2.93 (s, 3H), 2.92-2.83 (m, 3H), 2.05-1.95 (m, 2H), 1.86-1.73 (m, 2H). |
| 69 | 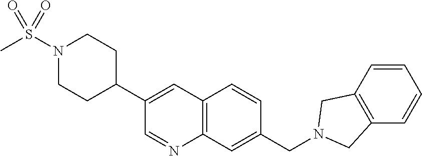 [SMILES: CS(=O)(=O)N1CCC(c2cnc3cc(CN4Cc5ccccc5C4)ccc3c2)CC1] | MS m/z: 422.1 (M + 1)⁺. ¹H NMR (400 MHz, CD₃OD) δ 9.32 (d, J = 2.0 Hz, 1H), 9.10 (s, 1H), 8.52 (s, 1H), 8.44 (d, J = 8.4 Hz, 1H), 8.12 (d, J = 8.4 Hz, 1H), 7.43 (s, 4H), 5.01 (s, 2H), 4.82 (s, 4H), 3.99-3.92 (m, 2H), 3.26-3.14 (m, 1H), 3.05-2.97 (m, 2H), 2.93 (s, 3H), 2.22-2.16 (m, 2H), 2.09-1.96 (m, 2H). |
| 70 | 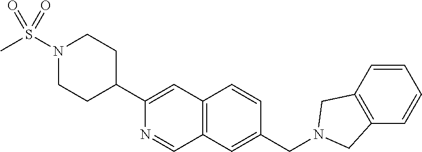 [SMILES: CS(=O)(=O)N1CCC(c2cc3ccc(CN4Cc5ccccc5C4)cc3cn2)CC1] | MS m/z: 422.2 (M + 1)⁺. ¹H NMR (500 MHz, CD₃OD) δ 9.87 (s, 1H), 8.82 (s, 1H), 8.51 (s, 1H), 8.42 (s, 2H), 7.41 (s, 4H), 5.01 (s, 2H), 4.83 (s, 4H), 3.97 (d, J = 12.0 Hz, 2H), 3.39-3.92 (m, 1H), 3.06-2.99 (m, 2H), 2.93 (s, 3H), 2.28 (d, J = 12.0 Hz, 2H), 2.16-1.97 (m, 2H). |
| 71 | 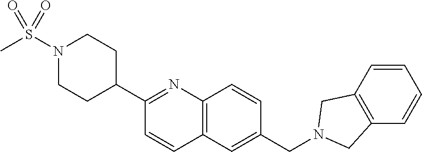 [SMILES: CS(=O)(=O)N1CCC(c2ccc3cc(CN4Cc5ccccc5C4)ccc3n2)CC1] | MS m/z: 422.1 (M + 1)⁺. ¹H NMR (400 MHz, CD₃OD) δ 9.18 (d, J = 8.8 Hz, 1H), 8.60 (d, J = 1.2 Hz, 1H), 8.47-8.43 (m, 1H), 8.40-8.36 (m, 1H), 8.20 (d, J = 8.8 Hz, 1H), 7.42 (s, 4H), 4.99 (s, 2H), 4.83 (s, 4H), 4.01 (d, J = 12.0 Hz, 2H), 3.54- 3.46 (m, 1H), 3.08-3.00 (m, 2H), 2.96 (s, 3H), 2.29-2.23 (m, 2H), 2.22-2.11 (m, 2H). |
| 72 | 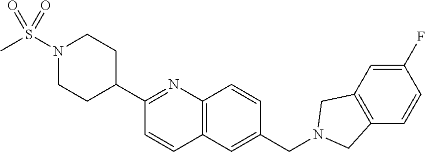 [SMILES: CS(=O)(=O)N1CCC(c2ccc3cc(CN4Cc5ccc(F)cc5C4)ccc3n2)CC1] | MS m/z: 440.3 (M + 1)⁺. ¹H NMR (400 MHz, CD₃OD) δ 9.20 (d, J = 8.8 Hz, 1H), 8.61 (d, J = 1.2 Hz, 1H), 8.47-8.35 (m, 2H), 8.21 (d, J = 8.8 Hz, 1H), 7.43 (dd, J = 4.8, 8.4 Hz, 1H), 7.28-7.11 (m, 2H), 4.99 (s, 2H), 4.80 (s, 4H), 4.01 (d, J = 12.0 Hz, 2H), 3.56-3.45 (m, 1H), 3.02 (dt, J = 2.4, 12.0 Hz, 2H), 2.95 (s, 3H), 2.24 (br s, 2H), 2.19-2.10 (m, 2H). |
| 73 | 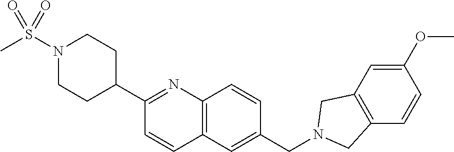 | MS m/z: 452.1 (M + 1)⁺. ¹H NMR (400 MHz, CD₃OD) δ 9.14 (d, J = 9.2 Hz, 1H), 8.56 (s, 1H), 8.41 (s, 1H), 8.34 (d, J = 8.8 Hz, 1H), 8.17 (d, J = 8.8 Hz, 1H), 7.30 (d, J = 9.2 Hz, 1H), 6.98 (s, 2H), 4.95 (s, 2H), 4.73 (s, 4H), 4.05-3.98 (m, 2H), 3.81 (s, 3H), 3.52-3.42 (m, 1H), 3.07-2.96 (m, 2H), 2.94 (s, 3H), 2.27-2.21 (m, 2H), 2.20-2.11 (m, 2H). |
| 74 | 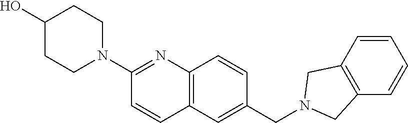 [SMILES: OC1CCN(c2ccc3cc(CN4Cc5ccccc5C4)ccc3n2)CC1] | MS m/z: 360.2 (M + 1)+. 1H NMR (500 MHz, CD3OD) δ 8.43 (d, J = 10.0 Hz, 1H), 8.17 (d, J = 1.5 Hz, 1H), 8.10-7.98 (m, 2H), 7.63 (d, J = 10.0 Hz, 1H), 7.41 (s, 4H), 4.81 (s, 2H), 4.76 (s, 4H), 4.24-4.17 (m, 2H), 4.10 (br s, 1H), 3.92- 3.80 (m, 2H), 2.15-2.08 (m, 2H), 1.84-1.74 (m, 2H). |
| 75 | 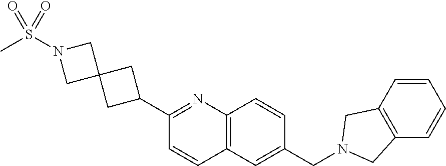 [SMILES: CS(=O)(=O)N1CC2(CC(c3ccc4cc(CN5Cc6ccccc6C5)ccc4n3)C2)C1] | MS m/z: 434.2 (M + 1)⁺. ¹H NMR (400 MHz, CDCl₃) δ 8.13-8.05 (m, 2H), 8.04-7.93 (m, 1H), 7.92-7.79 (m, 1H), 7.36-7.28 (m, 1H), 7.24 (s, 4H), 4.39-4.11 (m, 6H), 4.08 (s, 2H), 4.00-3.97 (m, 2H), 3.78-3.66 (m, 1H), 2.88 (s, 3H), 2.72-2.64 (m, 4H). |
| 76 | 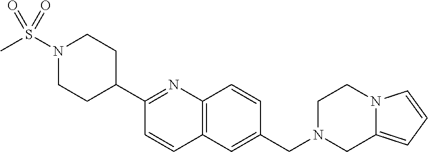 [SMILES: CS(=O)(=O)N1CCC(c2ccc3cc(CN4CCn5cccc5C4)ccc3n2)CC1] | MS m/z: 425.3 (M + 1)⁺. ¹H NMR (400 MHz, CD₃OD) δ 9.24 (d, J = 8.8 Hz, 1H), 8.63 (d, J = 1.2 Hz, 1H), 8.47 (s, 1H), 8.42-8.35 (m, 1H), 8.23 (d, J = 8.8 Hz, 1H), 6.79 (d, J = 2.7 Hz, 1H), 6.17 (d, J = 3.6 Hz, 1H), 6.02 (d, J = 3.6 Hz, 1H), 4.87- 4.81 (m, 2H), 4.52 (br s, 2H), 4.37 (br s, 2H), 3.99 (d, J = 12.0 Hz, 2H), 3.92 (d, J = 3.2 Hz, 2H), 3.60-3.49 (m, 1H), 3.03 (d, J = 2.4 Hz, 2H), 2.94 (s, 3H), 2.25 (br s, 2H), 2.21-2.10 (m, 2H). |
| 77 | 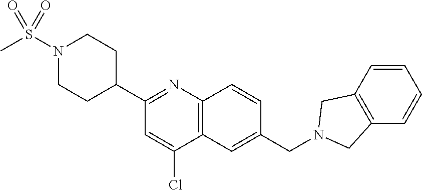 [SMILES: CS(=O)(=O)N1CCC(c2cc(Cl)c3cc(CN4Cc5ccccc5C4)ccc3n2)CC1] | MS m/z: 456.1 (M + 1)⁺. ¹H NMR (400 MHz, CD₃OD) δ 8.69 (d, J = 1.6 Hz, 1H), 8.33 (d, J = 8.8 Hz, 1H), 8.25-8.17 (m, 1H), 8.11 (s, 1H), 7.41 (s, 4H), 4.96 (s, 2H), 4.79 (s, 4H), 3.94 (br d, J = 12.0 Hz, 2H), 3.28-3.23 (m, 1H), 3.01-2.94 (m, 2H), 2.90 (s, 3H), 2.22-2.15 (m, 2H), 2.14- 2.04 (m, 2H). |
| 78 | 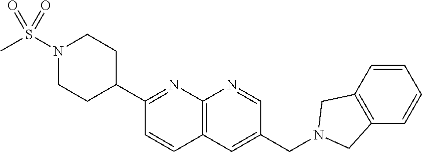 [SMILES: CS(=O)(=O)N1CCC(c2ccc3cc(CN4Cc5ccccc5C4)cnc3n2)CC1] | MS m/z: 423.2 (M + 1)⁺. ¹H NMR (400 MHz, CD₃OD) δ 9.10 (d, J = 2.4 Hz, 1H), 8.45-8.36 (m, 2H), 7.65 (d, J = 8.4 Hz, 1H), 7.20 (s, 4H), 4.19 (s, 2H), 4.01 (s, 4H), 3.91 (d, J = 11.6 Hz, 2H), 3.19- 3.12 (m, 1H), 3.01-2.94 (m, 2H), 2.90 (s, 3H), 2.18-2.06 (m, 4H). |
| 79 | 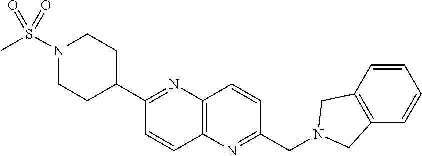 [SMILES: CS(=O)(=O)N1CCC(c2ccc3nc(CN4Cc5ccccc5C4)ccc3n2)CC1] | MS m/z: 423.1 (M + 1)⁺. ¹H NMR (400 MHz, CD₃OD) δ 9.03 (d, J = 8.8 Hz, 1H), 8.73 (d, J = 8.8 Hz, 1H), 8.25 (d, J = 8.8 Hz, 1H), 8.07 (d, J = 8.8 Hz, 1H), 7.45 (s, 4H), 5.23 (s, 2H), 4.92- 4.91 (m, 4H), 3.97 (d, J = 12.0 Hz, 2H), 3.50-3.39 (m, 1H), 3.06-2.97 (m, 2H), 2.93 (s, 3H), 2.27- 2.20 (m, 2H), 2.19-2.08 (m, 2H). |
| 80 | 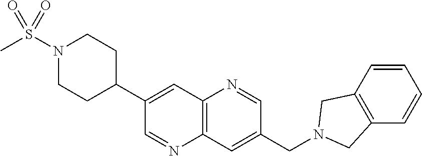 [SMILES: CS(=O)(=O)N1CCC(c2cnc3cc(CN4Cc5ccccc5C4)cnc3c2)CC1] | MS m/z: 423.2 (M + 1)⁺. ¹H NMR (400 MHz, CD₃OD) δ 9.38 (d, J = 2.0 Hz, 1H), 9.32-9.23 (m, 1H), 8.97 (s, 1H), 8.73-8.58 (m, 1H), 7.42 (s, 4H), 5.07 (s, 2H), 4.86 (s, 4H), 3.95 (d, J = 12.0 Hz, 2H), 3.25-3.17 (m, 1H), 3.03-2.96 (m, 2H), 2.93 (s, 3H), 2.20-2.14 (m, 2H), 2.06-1.95 (m, 2H). |
| 81 | 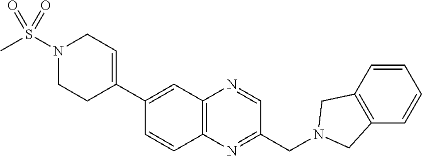 [SMILES: CS(=O)(=O)N1CC=C(c2ccc3nc(CN4Cc5ccccc5C4)cnc3c2)CC1] | MS m/z: 421.0 (M + 1)⁺. ¹H NMR (400 MHz, CD₃OD) δ 8.96 (s, 1H), 8.20-8.12 (m, 3H), 7.46 (s, 4H), 6.57-6.53 (m, 1H), 5.14 (s, 2H), 4.97 (s, 4H), 4.08-4.03 (m, 2H), 3.60-3.57 (m, 2H), 2.89 (s, 3H), 2.76-2.64 (m, 2H). |
| 82 | 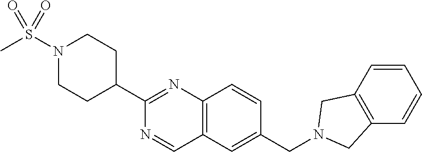 [SMILES: CS(=O)(=O)N1CCC(c2ncc3cc(CN4Cc5ccccc5C4)ccc3n2)CC1] | MS m/z: 423.1 (M + 1)⁺. ¹H NMR (400 MHz, CD₃OD) δ 7.88-7.83 (m, 2H), 7.57 (d, J = 8.0 Hz, 1H), 7.40 (s, 4H), 6.23 (s, 1H), 4.79-4.66 (m, 6H), 3.96-3.94 (m, 2H), 3.11-3.08 (m, 1H), 2.99-2.93 (m, 2H), 2.92 (s, 3 H), 2.19-2.15 (m, 2H), 2.07-2.04 (m, 2H). |
| 83 | 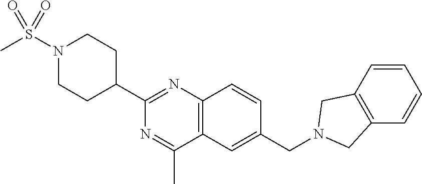 [SMILES: Cc1nc(C2CCN(S(C)(=O)=O)CC2)nc2ccc(CN3Cc4ccccc4C3)cc12] | MS m/z: 436.6 (M + 1)⁺. ¹H NMR (400 MHz, CD₃OD) δ 8.28 (s, 1H), 8.08-7.93 (m, 2H), 7.23 (d, J = 2.0 Hz, 4H), 4.81-4.79 (m, 2H), 4.20 (br s, 2H), 4.03 (s, 4H), 3.92-3.84 (m, 2H), 3.18-3.08 (m, 1H), 2.98 (s, 3H), 2.89 (s, 3H), 2.20-2.09 (m, 4H). |
| 84 | 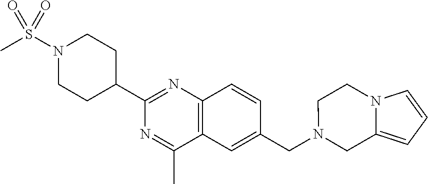 [SMILES: Cc1nc(C2CCN(S(C)(=O)=O)CC2)nc2ccc(CN3CCn4cccc4C3)cc12] | MS m/z 440.1 (M + 1)⁺. ¹H NMR (400 MHz, CD₃OD) δ 8.22 (s, 1H), 8.04-7.99 (m, 1H), 7.96- 7.91 (m, 1H), 6.62-6.52 (m, 1H), 6.02 (t, J = 3.2 Hz, 1H), 5.72 (d, J = 2.0 Hz, 1H), 4.60 (br s, 2H), 4.00 (t, J = 5.6 Hz, 2H), 3.92 (s, 2H), 3.87 (d, J = 12.0 Hz, 2H), 3.67 (s, 2H), 2.96 (s, 3H), 2.92 (t, J = 5.6 Hz, 3H), 2.88 (s, 3H), 2.19-2.10 (m, 4H). |
| 85 | 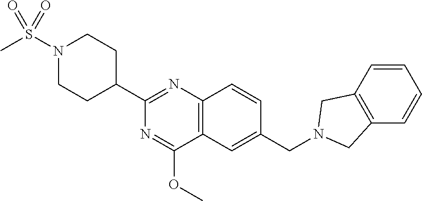 [SMILES: COc1nc(C2CCN(S(C)(=O)=O)CC2)nc2ccc(CN3Cc4ccccc4C3)cc12] | MS m/z: 453.2 (M + 1)⁺. ¹H NMR (400 MHz, CD₃OD) δ 8.23 (s, 1H), 8.04-7.95 (m, 1H), 7.89 (d, J = 8.4 Hz, 1H), 7.20 (s, 4H), 4.24 (s, 3H), 4.12 (s, 2H), 3.98 (s, 4H), 3.88 (d, J = 11.6 Hz, 2H), 3.09-2.94 (m, 3H), 2.90 (s, 3H), 2.22-2.10 (m, 4H). |
| 86 | 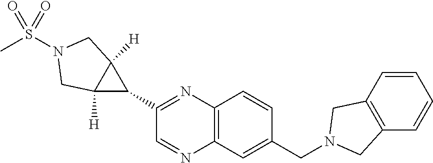 [SMILES: CS(=O)(=O)N1C[C@@H]2[C@H](C1)[C@H]2c1cnc2cc(CN3Cc4ccccc4C3)ccc2n1] | MS m/z: 421.1 (M + 1)⁺. ¹H NMR (400 MHz, CDCl₃) δ 8.85 (s, 1H), 8.09-8.02 (m, 2H), 8.01- 7.94 (m, 1H), 7.41-7.37 (m, 2H), 7.34-7.29 (m, 2H), 4.93-4.87 (m, 2H), 4.61 (s, 2H), 4.48-4.43 (m, 2H), 3.86 (d, J = 9.6 Hz, 2H), 3.53 (d, J = 9.6 Hz, 2H), 2.93 (s, 3H), 2.49-2.43 (m, 3H). |
| 87 | 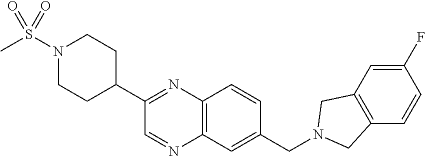 [SMILES: CS(=O)(=O)N1CCC(c2cnc3cc(CN4Cc5ccc(F)cc5C4)ccc3n2)CC1] | MS m/z: 441.1 (M + 1)⁺. ¹H NMR (400 MHz, CD₃OD) δ 8.99 (s, 1H), 8.33 (d, J = 1.6 Hz, 1H), 8.23 (d, J = 8.4 Hz, 1H), 7.99 (dd, J = 2.0, 8.4 Hz, 1H), 7.42 (dd, J = 4.8, 8.4 Hz, 1H), 7.23- 7.09 (m, 2H), 4.91 (s, 2H), 4.00 (s, 4H), 3.93 (d, J = 12.0 Hz, 2H), 3.29-3.20 (m, 1H), 2.99 (dt, J = 2.6, 12.0 Hz, 2H), 2.91 (s, 3H), 2.23-2.14 (m, 2H), 2.13-2.01 (m, 2H). |
| 88 | 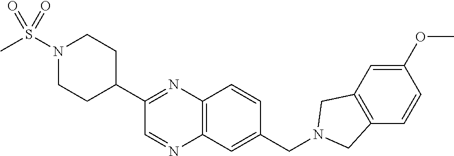 | MS m/z: 451.2 (M + 1)⁺. ¹H NMR (400 MHz, DMSO-d6) δ 9.05 (s, 1H), 8.42 (s, 1H), 8.17-8.16 (m, 2H), 7.28-7.26 (m, 1H), 7.00-6.96 (m, 1H), 6.93-6.89 (m, 1H), 4.86-4.85 (m, 2H), 4.60-4.53 (m, 4H), 3.75-3.74 (m, 2H), 3.72 (s, 3H), 3.21- 3.17 (m, 1H), 2.93 (s, 3H), 2.90-2.87 (m, 2H), 2.13-2.10 (m, 2H), 1.95-1.89 (m, 2H). |
| 89 | 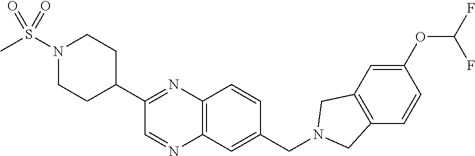 [SMILES: CS(=O)(=O)N1CCC(c2cnc3cc(CN4Cc5ccc(OC(F)F)cc5C4)ccc3n2)CC1] | MS m/z: 489.1 (M + 1)⁺. ¹H NMR (400 MHz, CD₃OD) δ 8.99 (s, 1H), 8.33 (s, 1H), 8.23 (d, J = 8.8 Hz, 1H), 8.00 (d, J = 8.4 Hz, 1H), 7.43 (d, J = 8.4 Hz, 1H), 7.25-7.16 (m, 2H), 7.07-6.64 (m, 1H), 4.92 (s, 2H), 4.78 (br s, 4H), 3.92 (d, J = 12.0 Hz, 2H), 3.28-3.21 (m, 1H), 2.99 (t, J = 12.0 Hz, 2H), 2.91 (s, 3H), 2.21-2.15 (m, 2H), 2.12-2.03 (m, 2H). |
| 90 | 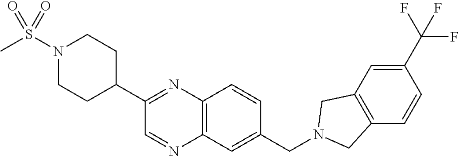 [SMILES: CS(=O)(=O)N1CCC(c2cnc3cc(CN4Cc5ccc(C(F)(F)F)cc5C4)ccc3n2)CC1] | MS m/z: 491.1 (M + 1)⁺. ¹H NMR (400 MHz, CD₃OD) δ 8.99 (s, 1H), 8.34 (s, 1H), 8.24-8.22 (m, 1H), 8.01-7.99 (m, 1H), 7.76-7.74 (m, 2H), 7.64-7.54 (m, 1H), 4.92 (s, 2H), 4.79 (s, 4H), 4.04-3.86 (m, 2H), 3.27-3.24 (m, 1H), 3.04-2.95 (m, 2H), 2.90 (s, 3H), 2.19-2.16 (m, 2H), 2.08- 2.04 (m, 2H). |
| 91 | 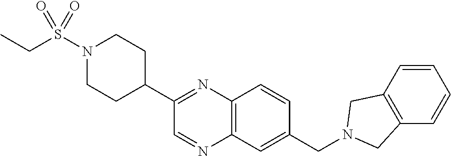 [SMILES: CCS(=O)(=O)N1CCC(c2cnc3cc(CN4Cc5ccccc5C4)ccc3n2)CC1] | MS m/z: 437.1 (M + 1)⁺. ¹H NMR (500 MHz, CD₃OD) δ 8.98 (s, 1H), 8.33 (d, J = 1.5 Hz, 1H), 8.24-8.20 (m, 1H), 8.00 (dd, J = 8.5, 2.0 Hz, 1H), 7.41 (s, 4H), 4.91 (s, 2H), 4.78 (s, 4H), 3.95 (d, J = 12.0 Hz, 2H), 3.29-3.24 (m, 1H), 3.13-3.07 (m, 4H), 2.16-2.12 (m, 2H), 2.08-2.00 (m, 2H), 1.37 (t, J = 7.2 Hz, 3H). |
| 92 | 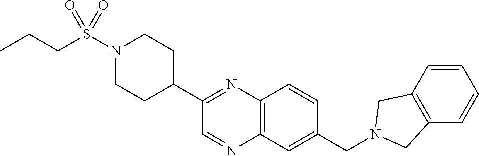 [SMILES: CCCS(=O)(=O)N1CCC(c2cnc3cc(CN4Cc5ccccc5C4)ccc3n2)CC1] | MS m/z: 451.2 (M + 1)⁺. ¹H NMR (500 MHz, CD₃OD) δ 8.98 (s, 1H), 8.33 (d, J = 1.5 Hz, 1H), 8.22 (d, J = 8.5 Hz, 1H), 8.00 (dd, J = 8.5, 1.5 Hz, 1H), 7.41 (s, 4H), 4.91 (s, 2H), 4.78 (s, 4H), 3.94 (d, J = 12.0 Hz, 2H), 3.29-3.23 (m, 1H), 3.10-3.03 (m, 4H), 2.19-2.12 (m, 2H), 2.09-1.99 (m, 2H), 1.91-1.81 (m, 2H), 1.10 (t, J = 7.5 Hz, 3H). |
| 93 | 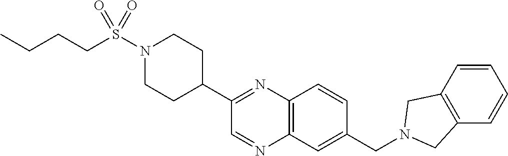 [SMILES: CCCCS(=O)(=O)N1CCC(c2cnc3cc(CN4Cc5ccccc5C4)ccc3n2)CC1] | MS m/z: 465.6 (M + 1)⁺. ¹H NMR (400 MHz, CD₃OD) δ 8.98 (s, 1H), 8.33 (d, J = 1.2 Hz, 1H), 8.22 (d, J = 8.4 Hz, 1H), 8.01 (d, J = 1.6 Hz, 1H), 7.41 (s, 4H), 4.91 (s, 2H), 4.78 (s, 4H), 3.94 (d, J = 12.0 Hz, 2H), 3.27-3.23 (m, 1H), 3.12-3.01 (m, 4H), 2.13 (br s, 2H), 2.09-2.00 (m, 2H), 1.85-1.76 (m, 2H), 1.60-1.48 (m, 2H), 1.00 (d, J = 7.2 Hz, 3H). |
| 94 | 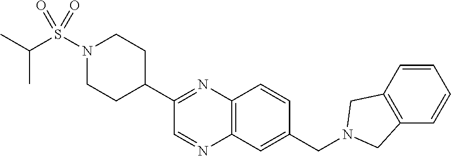 [SMILES: CC(C)S(=O)(=O)N1CCC(c2cnc3cc(CN4Cc5ccccc5C4)ccc3n2)CC1] | MS m/z: 451.2 (M + 1)⁺. ¹H NMR (400 MHz, CD₃OD) δ 8.99 (s, 1H), 8.35 (d, J = 1.6 Hz, 1H), 8.22 (d, J = 8.8 Hz, 1H), 8.04 (dd, J = 2.0, 8.8 Hz, 1H), 7.41 (s, 4H), 4.92 (s, 2H), 4.78 (s, 4H), 3.99-3.96 (m, 2H), 3.37-3.32 (m, 2H), 3.20-3.14 (m, 2H), 2.15-1.98 (m, 4H), 1.38 (d, J = 6.8 Hz, 6H). |
| 95 | 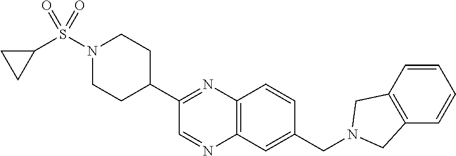 [SMILES: O=S(=O)(C1CC1)N1CCC(c2cnc3cc(CN4Cc5ccccc5C4)ccc3n2)CC1] | MS m/z: 449.2 (M + 1)⁺. ¹H NMR (400 MHz, CD₃Cl) δ 8.91 (s, 1H), 8.67-8.53 (m, 1H), 8.34- 8.18 (m, 2H), 7.45-7.37 (m, 2H), 7.34-7.29 (m, 2H), 4.99 (dd, J = 14.6, 4.8 Hz, 2H), 4.63 (d, J = 5.2 Hz, 2H), 4.30 (d, J = 12.8 Hz, 2H), 4.05 (d, J = 12.0 Hz, 2H), 3.27-3.14 (m, 1H), 3.14-3.02 (m, 2H), 2.42-2.31 (m, 1H), 2.22-2.11 (m, 4H), 1.28-1.23 (m, 2H), 1.11-1.02 (m, 2H). |
| 96 | 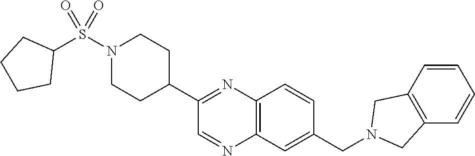 [SMILES: O=S(=O)(C1CCCC1)N1CCC(c2cnc3cc(CN4Cc5ccccc5C4)ccc3n2)CC1] | MS m/z: 477.2 (M + 1)⁺. ¹H NMR (400 MHz, CD₃OD) δ 8.99 (s, 1H), 8.34 (s, 1H), 8.25 (d, J = 8.4 Hz, 1H), 8.01 (d, J = 1.6 Hz, 1H), 7.43 (s, 4H), 4.93 (s, 2H), 4.80 (s, 4H), 4.02-3.95 (m, 2H), 3.74-3.65 (m, 1H), 3.29-3.27 (m, 1H), 3.17-3.11 (m, 2H), 2.17-2.05 (m, 5H), 2.04-1.96 (m, 3H), 1.85-1.79 (m, 2H), 1.74-1.65 (m, 2H). |
| 97 | 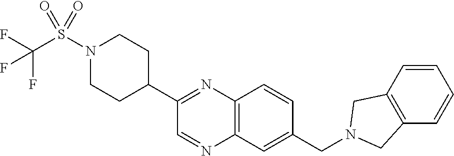 [SMILES: O=S(=O)(N1CCC(c2cnc3cc(CN4Cc5ccccc5C4)ccc3n2)CC1)C(F)(F)F] | MS m/z: 477.1 (M + 1)⁺. ¹H NMR (400 MHz, CD₃OD) δ 8.97 (s, 1H), 8.32 (d, J = 1.6 Hz, 1H), 8.23 (d, J = 8.4 Hz, 1H), 7.97 (dd, J = 2.0, 8.7 Hz, 1H), 7.41 (s, 4H), 4.90 (s, 2H), 4.77 (s, 4H), 4.10 (d, J = 13.2 Hz, 2H), 3.43-3.39 (m, 1H), 3.39-3.35 (m, 2H), 2.26-2.14 (m, 2H), 2.11-1.98 (m, 2H). |
| 98 | 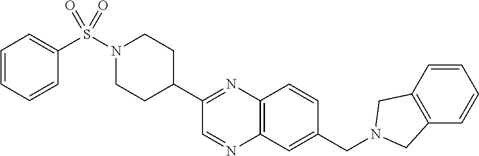 [SMILES: O=S(=O)(c1ccccc1)N1CCC(c2cnc3cc(CN4Cc5ccccc5C4)ccc3n2)CC1] | MS m/z: 485.1 (M + 1)⁺. ¹H NMR (500 MHz, CD₃OD) δ 8.92 (s, 1H), 8.32 (d, J = 1.5 Hz, 1H), 8.21 (d, J = 8.5 Hz, 1H), 8.00 (dd, J = 8.5, 1.5 Hz, 1H), 7.90-7.84 (m, 2H), 7.73 (d, J = 7.5 Hz, 1H), 7.70-7.65 (m, 2H), 7.43 (s, 4H), 4.92 (s, 2H), 4.79 (s, 4H), 3.98 (d, J = 11.5 Hz, 2H), 3.08 (tt, J = 11.5, 4.0 Hz, 1H), 2.57 (td, J = 11.5, 2.5 Hz, 2H), 2.19-1.99 (m, 4H). |
| 99 | 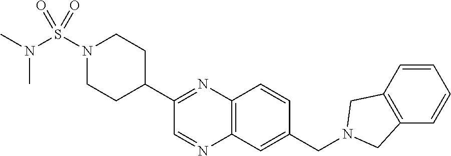 [SMILES: CN(C)S(=O)(=O)N1CCC(c2cnc3cc(CN4Cc5ccccc5C4)ccc3n2)CC1] | MS m/z: 452.2 (M + 1)⁺. ¹H NMR (400 MHz, CD₃Cl) δ 8.87 (s, 1H), 8.60 (d, J = 8.0 Hz, 1 H), 8.26-8.16 (m, 2 H) 7.41-7.37 (m, 2 H), 7.30-7.28 (m, 2 H), 4.97 (dd, J = 14.2, 5.2 Hz, 2 H), 4.60 (d, J = 5.6 Hz, 2H), 4.34 (dd, J = 14.2, 2.8 Hz, 2H), 3.89 (d, J = 12.4 Hz, 2H), 3.19-3.12 (m, 1 H), 3.07-3.00 (m, 2 H), 2.89-2.87 (s, 6 H), 2.07 (m, 4 H). |
| 100 | 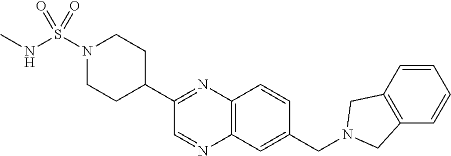 [SMILES: CNS(=O)(=O)N1CCC(c2cnc3cc(CN4Cc5ccccc5C4)ccc3n2)CC1] | MS m/z: 438.1 (M + 1)⁺. ¹H NMR (500 MHz, CD₃OD) δ 8.89 (s, 1H), 8.14-8.05 (m, 2H), 7.94 (dd, J = 2.0, 8.5 Hz, 1H), 7.22 (s, 4H), 4.19 (s, 2H), 4.01 (s, 4H), 3.86 (d, J = 12.0 Hz, 2H), 3.24-3.18 (m, 1H), 3.01 (dt, J = 2.5, 12.0 Hz, 2H), 2.71 (s, 3H), 2.17-2.06 (m, 4H). |
| 101 | 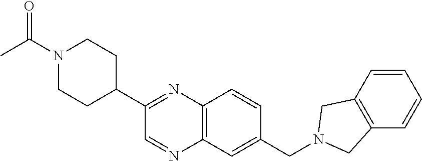 [SMILES: CC(=O)N1CCC(c2cnc3cc(CN4Cc5ccccc5C4)ccc3n2)CC1] | MS m/z: 387.1 (M + 1)⁺. ¹H NMR (400 MHz, CD₃OD) δ 9.00 (s, 1H), 8.35 (d, J = 1.6 Hz, 1H), 8.21 (d, J = 8.4 Hz, 1H), 8.04 (dd, J = 2.0, 8.8 Hz, 1H), 7.41 (s, 4H), 4.92 (s, 2H), 4.78 (s, 4H), 4.71 (d, J = 13.2 Hz, 1H), 4.25-4.05 (m, 1H), 3.42 (td, J = 4.0, 7.6 Hz, 2H), 3.04-2.84 (m, 1H), 2.24 (s, 3H), 2.19-2.09 (m, 2H), 2.06- 1.84 (m, 2H). |
| 102 | 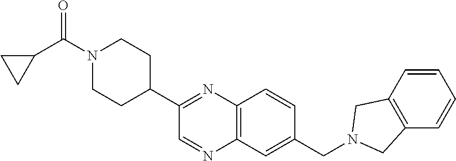 [SMILES: O=C(C1CC1)N1CCC(c2cnc3cc(CN4Cc5ccccc5C4)ccc3n2)CC1] | MS m/z: 413.1 (M + 1)⁺. ¹H NMR (500 MHz, CD₃OD) δ 8.99 (s, 1H), 8.32 (d, J = 1.5 Hz, 1H), 8.22 (d, J = 8.5 Hz, 1H), 7.98 (dd, J = 8.5, 2.0 Hz, 1H), 7.40 (s, 4H), 4.90 (s, 2H), 4.78 (s, 4H), 4.72-4.66 (m, 1H), 4.53 (br s, 1H), 3.46-3.40 (m, 2H), 2.98-2.87 (m, 1H), 2.20-2.14 (m, 1H), 2.10-1.99 (m, 3H), 1.91-1.82 (m, 1H), 0.93-0.90 (m, 2H), 0.87-0.83 (m, 2H). |
| 103 | 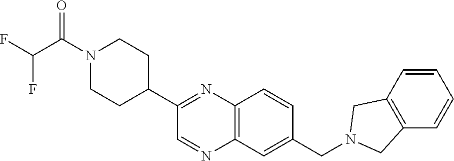 [SMILES: O=C(C(F)F)N1CCC(c2cnc3cc(CN4Cc5ccccc5C4)ccc3n2)CC1] | MS m/z: 423.1 (M + 1)⁺. ¹H NMR (500 MHz, CD₃OD) δ 8.87 (s, 1H), 8.10-8.05 (m, 2H), 7.92 (dd, J = 8.5, 1.5 Hz, 1H), 7.21 (s, 4H), 6.54 (t, J = 5.0 Hz, 1H), 4.58 (s, 2H), 4.17 (s, 2H), 4.00 (s, 4H), 3.45-3.40 (m, 2H), 3.08-3.02 (m, 1H), 2.18-2.13 (m, 2H), 2.05-1.93 (m, 2H). |
| 104 | 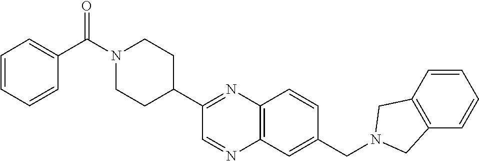 [SMILES: O=C(c1ccccc1)N1CCC(c2cnc3cc(CN4Cc5ccccc5C4)ccc3n2)CC1] | MS m/z: 449.2 (M + 1)⁺. ¹H NMR (400 MHz, CD₃OD) δ 9.00 (s, 1H), 8.34 (d, J = 1.6 Hz, 1H), 8.23 (d, J = 8.4 Hz, 1H), 8.03 (dd, J = 8.4, 1.97 Hz, 1H), 7.52-7.46 (m, 5H), 7.41 (s, 4H), 4.92 (s, 2H), 4.78 (s, 4H), 4.00-3.87 (m, 1H), 3.51-3.36 (m, 2H), 3.32 (br s, 2H), 3.19-3.06 (m, 1H), 2.23-1.95 (m, 3H). |
| 105 | 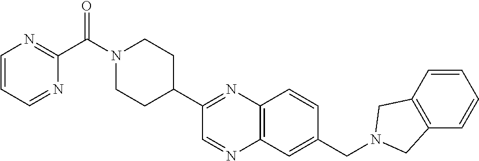 [SMILES: O=C(c1ncccn1)N1CCC(c2cnc3cc(CN4Cc5ccccc5C4)ccc3n2)CC1] | MS m/z: 451.4 (M + 1)⁺. ¹H NMR (400 MHz, CD₃OD) δ 9.02 (s, 1H), 8.95 (d, J = 4.0 Hz, 2H), 8.36 (s, 1H), 8.25 (d, J = 8.4 Hz, 1H), 8.03 (d, J = 8.4 Hz, 1H), 7.62 (t, J = 5.2 Hz, 1H), 7.43 (s, 4H), 4.94 (br s, 2H), 4.80 (s, 4H), 3.72-3.66 (m, 1H), 3.55-3.45 (m, 2H), 3.29-3.12 (m, 2H), 2.29- 2.22 (m, 1H), 2.16-2.04 (m, 3H). |
| 106 | 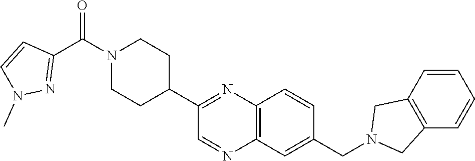 [SMILES: Cn1ccc(C(=O)N2CCC(c3cnc4cc(CN5Cc6ccccc6C5)ccc4n3)CC2)n1] | MS m/z: 453.3 (M + 1)⁺. ¹H NMR (400 MHz, CD₃OD) δ 9.05 (s, 1H), 8.38 (d, J = 1.6 Hz, 1H), 8.24 (d, J = 8.8 Hz, 1H), 8.08 (dd, J = 2.0, 8.8 Hz, 1H), 7.70 (d, J = 2.0 Hz, 1H), 7.41 (s, 4H), 6.65 (d, J = 2.4 Hz, 1H), 4.83 (s, 2H), 4.79 (s, 4H), 3.97 (s, 3H), 3.72-3.32 (m, 3H), 3.30-2.96 (m, 2H), 2.15 (d, J = 4.4 Hz, 2H), 2.10-2.00 (m, 2H). |
| 107 | 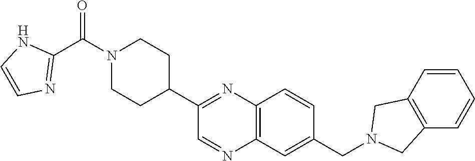 [SMILES: O=C(c1ncc[nH]1)N1CCC(c2cnc3cc(CN4Cc5ccccc5C4)ccc3n2)CC1] | MS m/z: 439.2 (M + 1)⁺. ¹H NMR (500 MHz, CD₃OD) δ 8.91 (s, 1H), 8.13-8.04 (m, 2H), 7.99- 7.90 (m, 1H), 7.27-7.15 (m, 6H), 5.62-5.30 (m, 1H), 4.84-4.79 (m, 1H), 4.19 (s, 2H), 4.01 (s, 4H), 3.50-3.40 (m, 2H), 3.20-3.11 (m, 1H), 2.20-2.04 (m, 4H). |
| 108 | 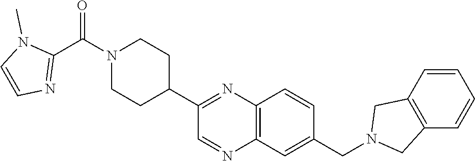 [SMILES: Cn1ccnc1C(=O)N1CCC(c2cnc3cc(CN4Cc5ccccc5C4)ccc3n2)CC1] | MS m/z: 453.2 (M + 1)⁺. ¹H NMR (500 MHz, CD₃OD) δ 9.03 (s, 1H), 8.37 (d, J = 1.5 Hz, 1H), 8.22 (d, J = 8.5 Hz, 1H), 8.06 (dd, J = 2.0, 8.5 Hz, 1H), 7.79-7.69 (m, 2H), 7.43 (s, 4H), 4.94 (s, 2H), 4.80 (s, 4H), 4.03 (s, 3H), 3.82-2.77 (m, 1H), 3.66-3.50 (m, 2H), 3.32-3.24 (m, 2H), 2.32-2.06 (m, 4H). |
| 109 | 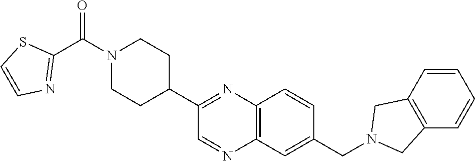 [SMILES: O=C(c1nccs1)N1CCC(c2cnc3cc(CN4Cc5ccccc5C4)ccc3n2)CC1] | MS m/z: 456.1 (M + 1)⁺. ¹H NMR (400 MHz, CD₃OD) δ 9.01 (s, 1H), 8.34 (d, J = 1.6 Hz, 1H), 8.22 (d, J = 8.4 Hz, 1H), 8.01 (dd, J = 8.4, 2.0 Hz, 1H), 7.98 (d, J = 3.2 Hz, 1H), 7.85 (d, J = 3.2 Hz, 1H), 7.42 (s, 4H), 4.92 (s, 2 H), 4.78 (s, 4H), 3.62-3.40 (m, 3H), 3.26-3.10 (m, 2H), 2.21-2.13 (m, 2H), 2.12-2.04 (m, 2H). |
| 110 | 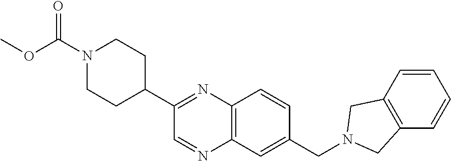 [SMILES: COC(=O)N1CCC(c2cnc3cc(CN4Cc5ccccc5C4)ccc3n2)CC1] | MS m/z: 403.1 (M + 1)⁺. ¹H NMR (400 MHz, CD₃OD) δ 9.00 (s, 1H), 8.36 (d, J = 1.6 Hz, 1H), 8.22 (d, J = 8.4 Hz, 1H), 8.08-8.01 (m, 1H), 7.41 (s, 4H), 4.97 (s, 2H), 4.78 (s, 4H), 4.35-4.28 (m, 2H), 3.73 (s, 3H), 3.40-3.32 (m, 1H), 3.18-2.95 (m, 2H), 2.13-2.05 (m, 2H), 1.98-1.83 (m, 2H). |
| 111 | 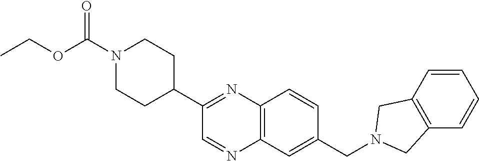 [SMILES: CCOC(=O)N1CCC(c2cnc3cc(CN4Cc5ccccc5C4)ccc3n2)CC1] | MS m/z: 417.3 (M + 1)⁺. ¹H NMR (400 MHz, CD₃OD) δ 9.02-8.94 (m, 1H), 8.32 (d, J = 1.6 Hz, 1H), 8.21 (d, J = 8.4 Hz, 1H), 7.99 (dd, J = 8.4, 1.6 Hz, 1H), 7.41 (s, 4H), 4.91 (s, 2H), 4.78 (s, 4H), 4.32 (d, J = 12.8 Hz, 2H), 4.17 (q, J = 7.2 Hz, 2H), 3.33 (br s, 1H), 3.12-3.01 (m, 2H), 2.07- 2.02 (m, 2H), 1.95-1.86 (m, 2H), 1.30 (t, J = 7.2 Hz, 3H). |
| 112 | 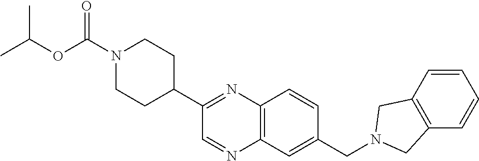 [SMILES: CC(C)OC(=O)N1CCC(c2cnc3cc(CN4Cc5ccccc5C4)ccc3n2)CC1] | MS m/z: 431.1 (M + 1)⁺. ¹H NMR (500 MHz, CD₃OD) δ 8.97 (s, 1H), 8.32 (d, J = 1.5 Hz, 1H), 8.21 (d, J = 8.5 Hz, 1H), 7.99 (dd, J = 8.5, 1.98 Hz, 1H), 7.41 (s, 4H), 4.92-4.89 (m, 3H), 4.78 (s, 4H), 4.31 (d, J = 13.5 Hz, 2H), 3.36-3.32 (m, 1 H), 3.05 (br s, 2H), 2.12-1.99 (m, 2H), 1.98-1.83 (m, 2H), 1.29 (d, J = 6.0 Hz, 6H). |
| 113 | 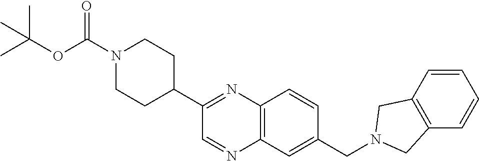 [SMILES: CC(C)(C)OC(=O)N1CCC(c2cnc3cc(CN4Cc5ccccc5C4)ccc3n2)CC1] | MS m/z: 445.2 (M + 1)⁺. ¹H NMR (400 MHz, CD₃OD) δ 8.86 (s, 1H), 8.07 (d, J = 10.0 Hz, 2H), 7.97-7.92 (m, 1H), 7.26-7.15 (m, 4H), 5.06-4.96 (m, 2H), 4.30-4.22 (m, 2H), 4.17 (s, 2H), 3.99 (s, 4H), 3.03-2.97 (m, 1H), 2.08-2.19 (m, 2H), 1.93-1.82 (m, 2H), 1.50 (s, 9H). |
| 114 | 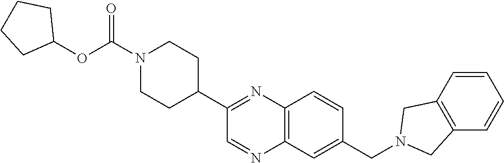 [SMILES: O=C(OC1CCCC1)N1CCC(c2cnc3cc(CN4Cc5ccccc5C4)ccc3n2)CC1] | MS m/z: 457.3 (M + 1)⁺. ¹H NMR (500 MHz, CD₃OD) δ 9.01-8.93 (m, 1H), 8.34-8.30 (m, 1H), 8.23-8.19 (m, 1H), 8.00-7.97 (m, 1H), 7.40 (s, 4H), 5.11 (t, J = 5.5 Hz, 1H), 4.90 (s, 2H), 4.77 (s, 4H), 4.33-4.24 (m, 2H), 3.11-2.98 (m, 2H), 2.09-2.00 (m, 2H), 1.94-1.82 (m, 4H), 1.80-1.71 (m, 5H), 1.70-1.61 (m, 2H). |
| 115 | 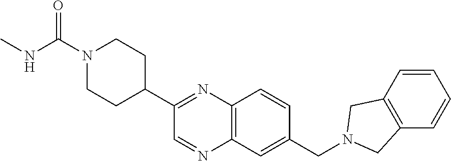 [SMILES: CNC(=O)N1CCC(c2cnc3cc(CN4Cc5ccccc5C4)ccc3n2)CC1] | MS m/z: 402.2 (M + 1)⁺. ¹H NMR (500 MHz, CD₃OD) δ 8.96 (s, 1H), 8.33-8.30 (m, 1H), 8.23- 8.19 (m, 1H), 8.01-7.97 (m, 1H), 7.41 (s, 4H), 4.91 (s, 2H), 4.78 (s, 4H), 4.22-4.18 (m, 2H), 3.36-3.33 (m, 1H), 3.06-3.00 (m, 2H), 2.76 (s, 3H), 2.06-2.01 (m, 2H), 1.96-1.86 (m, 2H). |
| 116 | 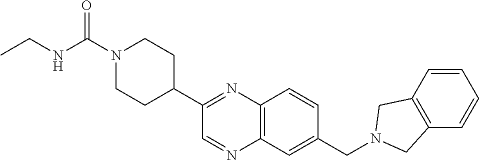 [SMILES: CCNC(=O)N1CCC(c2cnc3cc(CN4Cc5ccccc5C4)ccc3n2)CC1] | MS m/z: 416.2 (M + 1)⁺. ¹H NMR (500 MHz, CD₃OD) δ 8.98-8.96 (m, 1H), 8.33-8.30 (m, 1H), 8.23-8.20 (m, 1H), 7.98 (d, J = 2.0, 8.5 Hz, 1H), 7.41 (s, 4H), 4.90 (s, 2H), 4.77 (s, 4H), 4.21 (d, J = 13.5 Hz, 2H), 3.23 (q, J = 7.2 Hz, 2H), 3.05- 2.98 (m, 2H), 2.06-2.01 (m, 2H), 1.95-1.86 (m, 2H), 1.35-1.26 (m, 1H), 1.15 (t, J = 7.2 Hz, 3H). |
| 117 | 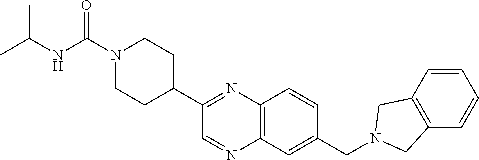 [SMILES: CC(C)NC(=O)N1CCC(c2cnc3cc(CN4Cc5ccccc5C4)ccc3n2)CC1] | MS m/z: 430.2 (M + 1)⁺. ¹H NMR (400 MHz, CD₃OD) δ 8.99 (s, 1H), 8.34 (d, J = 1.6 Hz, 1H), 8.22 (d, J = 8.8 Hz, 1H), 8.07-8.79 (m, 1H), 7.41 (s, 4H), 4.92 (s, 2H), 4.78 (s, 4H), 4.21 (br s, 1H), 3.94 (quin, J = 6.4 Hz, 1H), 3.43-3.32 (m, 2H), 3.14-2.93 (m, 2H), 2.14-2.00 (m, 2H), 1.98-1.82 (m, 2H), 1.18 (d, J = 6.4 Hz, 6H). |
| 118 | 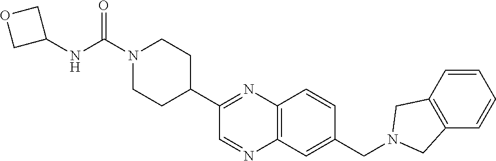 [SMILES: O=C(NC1COC1)N1CCC(c2cnc3cc(CN4Cc5ccccc5C4)ccc3n2)CC1] | MS m/z: 444.2 (M + 1)⁺. ¹H NMR (500 MHz, CD₃OD) δ 9.21 (s, 1H), 8.45 (s, 1H), 8.26 (d, J = 8.5 Hz, 1H), 8.14 (d, J = 8.5 Hz, 1H), 7.40 (s, 4H), 4.98-4.88 (m, 3H), 4.84-4.74 (m, 5H), 4.45- 4.39 (m, 1H), 4.30-4.24 (m, 1H), 4.10-4.04 (m, 1H), 3.81-3.73 (m, 1H), 3.71-3.65 (m, 1H), 3.63- 3.50 (m, 2H), 3.49-3.41 (m, 1H), 2.38-1.99 (m, 4H). |
| 119 | 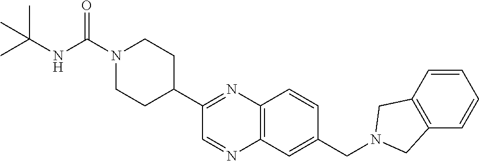 [SMILES: CC(C)(C)NC(=O)N1CCC(c2cnc3cc(CN4Cc5ccccc5C4)ccc3n2)CC1] | MS m/z: 444.2 (M + 1)⁺. ¹H NMR (400 MHz, CD₃OD) δ 9.00-8.96 (m, 1H), 8.36-8.32 (m, 1H), 8.24-8.19 (m, 1H), 8.03 (d, J = 2.0, 8.8 Hz, 1H), 7.41 (s, 4H), 4.93-4.91 (m, 2H), 4.78 (s, 4H), 4.21-4.13 (m, 2H), 3.37-3.32 (m, 1H), 3.06-2.96 (m, 2H), 2.10-2.01 (m, 2H), 1.99-1.87 (m, 2H), 1.39-1.36 (m, 9H). |
| 120 | 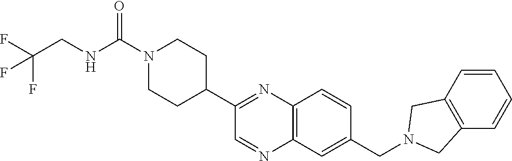 [SMILES: O=C(NCC(F)(F)F)N1CCC(c2cnc3cc(CN4Cc5ccccc5C4)ccc3n2)CC1] | MS m/z: 470.2 (M + 1)⁺. ¹H NMR (500 MHz, CD₃OD) δ 8.87 (s, 1H), 8.10-8.05 (m, 2H), 7.94- 7.91 (m, 1H), 7.22 (s, 4H), 4.29-4.20 (m, 2H), 4.17 (s, 2H), 4.00 (s, 4H), 3.93-3.86 (m, 2H), 3.30-3.26 (m, 1H), 3.12-3.04 (m, 2H), 2.09-2.03 (m, 2H), 1.98-1.89 (m, 2H). |
| 121 | 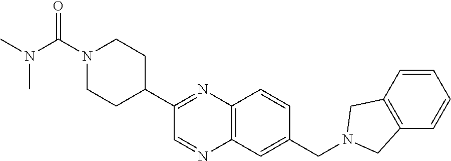 [SMILES: CN(C)C(=O)N1CCC(c2cnc3cc(CN4Cc5ccccc5C4)ccc3n2)CC1] | MS m/z: 416.2 (M + 1)⁺. ¹H NMR (400 MHz, CD₃OD) δ 8.97 (s, 1H), 8.32 (d, J = 1.6 Hz, 1H), 8.21 (d, J = 8.4 Hz, 1H), 7.98 (dd, J = 2.0, 8.4 Hz, 1H), 7.41 (s, 4H), 4.90 (s, 2H), 4.78 (s, 4H), 3.89- 3.83 (m, 2H), 3.30-3.24 (m, 1H), 3.09-2.99 (m, 2H), 2.90 (s, 6H), 2.09-2.03 (m, 2H), 2.03-1.95 (m, 2H). |
| 122 | 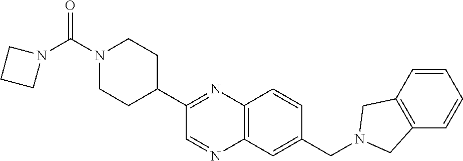 [SMILES: O=C(N1CCC1)N1CCC(c2cnc3cc(CN4Cc5ccccc5C4)ccc3n2)CC1] | MS m/z: 446.2 (M + 1)⁺. ¹H NMR (400 MHz, CD₃OD) δ 9.00 (s, 1H), 8.35 (d, J = 1.6 Hz, 1H), 8.21 (s, 1H), 8.09-7.98 (m, 1H), 7.41 (s, 4H), 4.92 (s, 2H), 4.78 (s, 4H), 4.25-4.18 (m, 2H), 3.65 (s, 1H), 3.63-3.58 (m, 2H), 3.38-3.35 (m, 2H), 3.06 (s, 2H), 2.11-2.04 (m, 2H), 2.02 (s, 2H), 1.97-1.89 (m, 2H). |
| 123 | 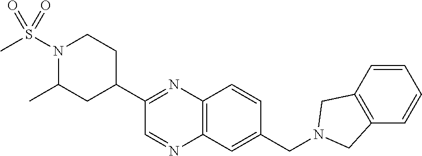 | MS m/z: 359.1 (M + 1)⁺. ¹H NMR (400 MHz, CD₃OD) δ 8.98 (s, 1H), 8.32 (d, J = 1.6 Hz, 1H), 8.21 (s, 1H), 8.00 (dd, J = 2.0, 8.4 Hz, 1H), 7.41 (s, 4H), 4.91 (s, 2H), 4.78 (s, 4H), 4.01-3.83 (m, 1H), 3.60 (br s, 1H), 3.39 (br s, 1H), 3.22 (br s, 1H), 2.97 (s, 3H), 2.24-2.08 (m, 3H), 2.05-1.96 (m, 1H), 1.37 (d, J = 6.4 Hz, 3H). |
| 124 | 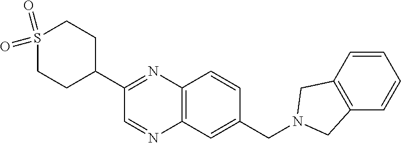 [SMILES: O=S1(=O)CCC(c2cnc3cc(CN4Cc5ccccc5C4)ccc3n2)CC1] | MS m/z: 394.2 (M + 1)⁺. ¹H NMR (400 MHz, CD₃OD) δ 8.99 (s, 1H), 8.40-8.31 (m, 1H), 8.26- 8.17 (m, 1H), 8.01 (dd, J = 1.6, 8.4 Hz, 1H), 7.41 (s, 4H), 4.93 (s, 2H), 4.80 (s, 4H), 3.55-3.47 (m, 1H), 3.43-3.35 (m, 2H), 3.28-3.21 (m, 2H), 2.63- 2.51 (m, 2H), 2.49-2.39 (m, 2H). |
| 125 | 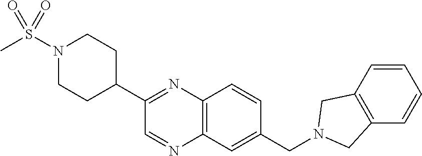 | MS m/z: 426.2 (M + 1)⁺. ¹H NMR (400 MHz, CD₃OD) δ 8.99 (s, 1H), 8.31 (s, 1H), 8.23 (d, J = 8.0 Hz, 1H), 7.96 (d, J = 8.4 Hz, 1H), 6.79 (s, 1H), 6.18 (d, J = 2.4 Hz, 1H), 6.04 (d, J = 1.2 Hz, 1H), 4.79 (br s, 2H), 4.50 (br s, 2H), 4.38-4.24 (m, 2H), 3.92 (d, J = 12.0 Hz, 4H), 3.25-3.19 (m, 1H), 3.03-2.95 (m, 2H), 2.95-2.88 (m, 3H), 2.20- 2.14 (m, 2H), 2.12-2.04 (m, 2H). |
| 126 | 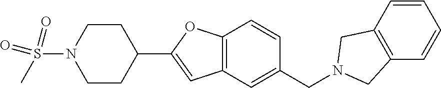 [SMILES: CS(=O)(=O)N1CCC(c2cc3cc(CN4Cc5ccccc5C4)ccc3o2)CC1] | MS m/z: 411.2 (M + 1)⁺. ¹H NMR (400 MHz, CD₃OD) δ 7.78 (s, 1H), 7.60 (d, J = 8.4 Hz, 1H), 7.46 (dd, J = 1.6, 8.4 Hz, 1H), 7.42 (s, 4H), 6.66 (s, 1H), 4.71-4.70 (m, 6H), 3.84-3.82 (m, 2H), 3.09-2.92 (m, 3H), 2.89 (s, 3H), 2.26-2.22 (m, 2H), 1.88-1.84 (m, 2H). |
| 127 | 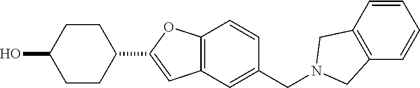 | MS m/z: 349.4 (M + 1)⁺. ¹H NMR (400 MHz, CD₃OD) δ 7.72 (d, J = 1.2 Hz, 1H), 7.57 (d, J = 8.3 Hz, 1H), 7.45 (dd, J = 1.6, 8.4 Hz, 1H), 7.31-7.13 (m, 4H), 4.04 (s, 2H), 3.94 (s, 4H), 3.77-3.59 (m, 1H), 3.07-2.90 (m, 1H), 2.37-2.22 (m, 2H), 2.19- 2.04 (m, 2H), 1.86-1.70 (m, 2H), 1.62-1.38 (m, 2H). |
| 128 | 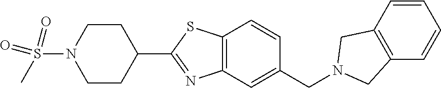 [SMILES: CS(=O)(=O)N1CCC(c2nc3cc(CN4Cc5ccccc5C4)ccc3s2)CC1] | MS m/z: 428.1 (M + 1)⁺. ¹H NMR (400 MHz, CD₃OD) δ 8.21-8.13 (m, 2H), 7.65-7.61 (m, 1H), 7.40 (s, 4H), 4.80 (s, 2H), 4.73 (s, 4H), 3.90-3.83 (m, 2H), 3.41-3.34 (m, 1H), 3.04-2.95 (m, 2H), 2.89 (s, 3H), 2.36-2.28 (m, 2H), 2.08-1.95 (m, 2H). |
| 129 | 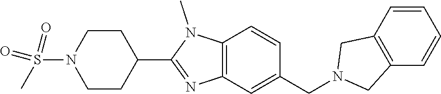 [SMILES: Cn1c(C2CCN(S(C)(=O)=O)CC2)nc2cc(CN3Cc4ccccc4C3)ccc21] | MS m/z: 425.2 (M + 1)⁺. ¹H NMR (400 MHz, CD₃OD) δ 7.69-7.59 (m, 1H), 7.58-7.48 (m, 1H), 7.43-7.31 (m, 1H), 7.27-7.18 (m, 4H), 4.10-4.02 (m, 2H), 3.96-3.93 (m, 4H), 3.90-3.88 (m, 2H), 3.86 (s, 3H), 3.29-3.20 (m, 1H), 3.04-2.96 (m, 2H), 2.92 (s, 3H), 2.17-2.02 (m, 4 H). |
| 130 | 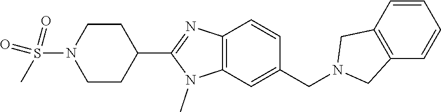 [SMILES: Cn1c(C2CCN(S(C)(=O)=O)CC2)nc2ccc(CN3Cc4ccccc4C3)cc21] | MS m/z: 425.3 (M + 1)⁺. ¹H NMR (400 MHz, CD₃OD) δ 7.67-7.58 (m, 1H), 7.57-7.47 (m, 1H), 7.40-7.30 (m, 1H), 7.24-7.16 (m, 4H), 4.09-4.02 (m, 2H), 3.95-3.91 (m, 4H), 3.88-3.86 (m, 2H), 3.82 (s, 3H), 3.24-3.21 (m, 1H), 2.98-2.90 (m, 2H), 2.90 (s, 3H), 2.15-2.01 (m, 4H). |
| 131 | 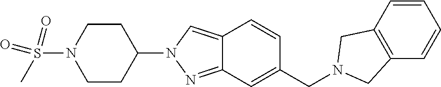 [SMILES: CS(=O)(=O)N1CCC(n2cc3ccc(CN4Cc5ccccc5C4)cc3n2)CC1] | MS m/z: 411.1 (M + 1)⁺. ¹H NMR (400 MHz, CD₃OD) δ 8.80-8.73 (m, 1H), 8.17-8.12 (m, 1H), 7.87-7.80 (m, 1H), 7.72-7.66 (m, 1H), 7.40 (s, 4H), 4.89-4.82 (m, 1H), 4.76 (s, 2H), 4.73 (s, 4H), 4.01-3.89 (m, 2H), 3.12-3.05 (m, 2H), 2.94 (s, 3H), 2.42-2.36 (m, 2H), 2.35-2.24 (m, 2H). |

### Example A: H295R Steroid Biosynthesis Inhibition Assay

Steroid Biosynthesis Inhibition by test compounds in NCI-H295R cells (ATCC) was studied by measuring the cellular production of pregnenolone (P) catalyzed by CYP11A1 and the downstream steroid, testosterone (T). Cells were cultured in Dulbecco's modified eagle's medium F12 (DMEM/F12) with phenol red (Gibco, Cat. No. 11330032) supplemented with 2% UltroserG (Pall, Cat. No. 15950-017, USA), 1% ITS-Premix and 1% penicillin and streptomycin (Hyclone, Cat. No. SV30010, USA). Prior to the assay, cells were thawed and passed 4 times. The maximal passage number used for the assay was 10. For this assay, DMEM/F12 without phenol red supplemented with 10% charcoal-stripped fetal bovine serum (VivaCell, Cat. No. C3830-0500, USA) were used. In a 96-well plate, 60,000 cells/well were plated (90 μL/well) and incubated for 24 h. Test compounds, diluted in a serial dilution ranging from 1000 nM to 0.15 nM in the assay medium, were added to the plate. After 48 h of incubation, solution from each well were collected and transferred to individual 1.5 mL centrifuge tubes, and then 5 ng/mL deuterated testosterone (D3-testosterone) and 50 ng/mL deuterated pregnenolone (D4-pregnenolone) in 200 μL acetonitrile were added. After vigorously mixing for 5 min, the mixture was centrifuged at 12,700 rpm for 15 min. An aliquot of 250 μL supernatant was collected and dried under N₂ stream. After addition of 150 μL Methanol:water (1:1, v/v) to the dried product, 50 μL sample of this solution was injected into the LC-MS/MS system for analysis.

The half-maximal inhibitory concentration (IC₅₀) for steroid biosynthesis inhibition was determined by curve-fitting with the 4-variable inhibitory dose response equation using GraphPad Prism software.

### LC-MS/MS Analysis of Steroid Hormones

Testosterone and pregnenolone were analyzed using Shimadzu LC30AD HPLC system (Shimadzu, Kyoto, Japan) coupled with AB SCIEX Qtrap 5500 triple quadrupole mass spectrometer (AB Sciex, Framingham, MA, USA) via ESI interface in the positive ionization mode. An ACQUITY UPLC BEH Shield RP18 column (1.7 μm, 2.1×50 mm, Milford, MA, USA) maintained at 40° C. was used to separate analytes and IS form the sample matrix.

The mobile phase was a mixture of 10 mM ammonium acetate in water containing 0.2% formic acid (A) and methanol containing 0.2% formic acid (B) at a flow rate of 0.6 mL/min. The gradient program was set as follows: from 0 min to 0.5 min, 60% B; from 0.5 min to 3.5 min, 90% B; from 3.5 min to 4.5 min, 90% B; from 4.51 min to 5.0 min, 60% B. The injection volume was 50 mL.

The MS/MS parameters were set as follows: ion spray voltage, 5500 V; curtain gas, 35 psi; Gas 1, 55 psi; Gas 2: 55 psi; temperature, 55° C. The optimized multiple reaction monitoring (MRM) fragmentation transitions and MS parameters of each analyte are summarized in Table 1.

TABLE 1
MS parameters for D3-testosterone and D4-pregnenolone

| | Precursor Ion | Product | Collision | Declustering |
|---|---|---|---|---|
| | [M + H]⁺ | Ion | Energy | Potential |
| Analyte | (m/z) | (m/z) | (eV) | (V) |
| Testosterone | 289.2 | 253.2 | 25 | 120 |
| | | 271.2 | 24 | 120 |
| D3-testosterone | 292.2 | 274.2 | 25 | 120 |
| Pregnenolone | 317.0 | 299.2 | 15 | 110 |
| | | 281.2 | 19 | 110 |
| | | 159.2 | 33 | 110 |
| D4-pregnenolone | 321.2 | 303.2 | 16 | 110 |

The compounds of the invention were screened in the above-mentioned assay and the IC₅₀ values of the compounds are set forth in Table 2 below, wherein “A” refers to IC₅₀≤40 nM, “B” refers to IC₅₀ value in the range of 41 to 100 nM, “C” refers to IC₅₀≥101 nM.

TABLE 2
Steroid Biosynthesis Inhibition Analysis

| Compound | Inh. of T | Inh. of P |
|---|---|---|
| No. | IC₅₀ (nM) | IC₅₀ (nM) |
| 1 | B | A |
| 2 | A | A |
| 3 | B | A |
| 4 | B | A |
| 5 | C | C |
| 6 | B | A |
| 7 | A | A |
| 8 | A | A |
| 9 | A | A |
| 10 | C | B |
| 11 | B | A |
| 12 | A | A |
| 13 | A | A |
| 14 | A | A |
| 15 | A | A |
| 16 | C | B |
| 17 | A | A |
| 18 | A | A |
| 19 | A | A |
| 20 | A | A |
| 21 | C | C |
| 22 | A | A |
| 23 | A | A |
| 24 | A | A |
| 25 | A | A |
| 26 | C | B |
| 27 | A | A |
| 28 | C | A |
| 29 | A | A |
| 30 | A | A |
| 31 | A | A |
| 32 | A | B |
| 33 | B | B |
| 34 | A | A |
| 35 | B | A |
| 36 | C | B |
| 37 | A | B |
| 38 | A | A |
| 39 | A | A |
| 40 | A | A |
| 41 | A | A |
| 42 | A | A |
| 43 | B | A |
| 44 | A | A |
| 45 | A | A |
| 46 | A | A |
| 47 | A | A |
| 48 | B | A |
| 49 | A | A |
| 50 | C | C |
| 51 | A | A |
| 52 | A | A |
| 53 | B | B |
| 54 | B | A |
| 55 | A | A |
| 56 | A | A |
| 57 | A | A |
| 58 | A | A |
| 59 | A | A |
| 60 | A | A |
| 61 | C | C |
| 62 | C | B |
| 63 | A | A |
| 64 | B | A |
| 65 | A | A |
| 66 | A | A |
| 67 | A | A |
| 68 | A | A |
| 69 | A | A |
| 60 | A | A |
| 71 | A | A |
| 72 | A | A |
| 73 | A | A |
| 74 | C | C |
| 75 | A | A |
| 76 | C | B |
| 77 | A | A |
| 78 | A | A |
| 79 | A | A |
| 80 | A | A |
| 81 | B | A |
| 82 | B | A |
| 83 | A | A |
| 84 | C | C |
| 85 | A | A |
| 86 | B | A |
| 87 | A | A |
| 88 | A | A |
| 89 | A | A |
| 90 | A | A |
| 91 | A | A |
| 92 | A | A |
| 93 | A | A |
| 94 | A | A |
| 95 | A | A |
| 96 | A | A |
| 97 | C | B |
| 98 | A | A |
| 99 | B | B |
| 100 | A | A |
| 101 | C | C |
| 102 | B | B |
| 103 | C | B |
| 104 | C | B |
| 105 | C | B |
| 106 | C | A |
| 107 | C | B |
| 108 | C | B |
| 109 | B | B |
| 110 | A | A |
| 111 | A | A |
| 112 | B | B |
| 113 | B | B |
| 114 | A | A |
| 115 | A | A |
| 116 | C | B |
| 117 | C | B |
| 118 | C | C |
| 119 | B | A |
| 120 | B | A |
| 121 | B | A |
| 122 | C | C |
| 123 | A | A |
| 124 | C | C |
| 125 | C | C |
| 126 | A | A |
| 127 | A | A |
| 128 | B | A |
| 129 | C | A |
| 130 | C | A |
| 131 | A | A |

1. A compound of Formula (I), 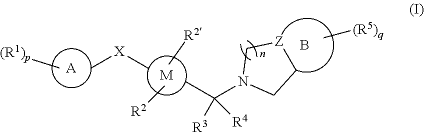
wherein
ring A is monocyclic, bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si) oxygen (O), sulfur (S) or nitrogen (N), and the ring-forming sulfur is optionally substituted with two oxo (═O) groups;
ring M is monocyclic or fused bicyclic ring, containing 0-4 ring-forming heteroatoms independently selected from N, O or S; wherein a ring-forming carbon atom is optionally substituted with oxo (═O) to form a carbonyl group;
ring B is 5- or 6-membered aryl or heteroaryl, and is optionally substituted with one to three R⁵;
X is —CR⁶R⁷—O— or a bond;
n is 1 or 2;
Z is Cor N;
each R¹ independently is H, C₁₋₇ alkyl, C₂₋₇ alkenyl, C₂₋₇ alkynyl, amino, alkylamino, dialkylamino, alkoxy, alkoxyalkyl, hydroxyalkyl, halogen, haloalkyl, —CN, cyanoalkyl, —OR¹¹, —SO₂R¹¹, —SO₂NR¹²R^{12′}, —NR¹²SO₂R¹¹, —C(═O)—R¹¹, —COOR¹¹, —COONR¹²R^{12′}, —C(═O)—NR¹²R^{12′}, —P(═O)R¹²R^{12′}, cycloalkyl, heterocyclyl, aryl or heteroaryl, wherein cycloalkyl, heterocyclyl, aryl or heteroaryl is optionally substituted with one to three R¹⁰;
each of R² and R^{2′} is independently H, halogen, haloalkyl, alkoxy, alkoxyalkyl, hydroxyl, hydroxyalkyl, —CN, cyanoalkyl, C₁₋₇ alkyl, cycloalkyl or heterocyclyl, wherein cycloalkyl or heterocyclyl is optionally substituted with one or more R¹⁰;
each of R³ and R⁴ independently is H, alkyl, cycloalkyl or heterocyclyl, and is optionally substituted with one or more substituents independently selected from halogen, haloalkyl, —OH, hydroxyalkyl, alkoxy or —CN;
or, R³ and R⁴, together with the atom to which they are both bonded, optionally form a 3-7 membered monocyclic, bicyclic or spirocyclic cycloalkyl or heterocyclyl, wherein cycloalkyl or heterocyclyl is optionally substituted with one to three R¹⁰;
each R⁵ independently is H, alkyl, halogen, haloalkyl, —OH, hydroxyalkyl, alkoxy, haloalkoxy, alkoxyalkyl, —CN, cyanoalkyl, amino, alkylamino, dialkylamino, aryl, heteroaryl, cycloalkyl, or heterocyclyl, wherein the aryl, heteroaryl, cycloalkyl or heterocyclyl is independently and optionally substituted with one or more R¹⁰;
each of R⁶ and R⁷ independently is H, alkyl, cycloalkyl or heterocyclyl, and is optionally substituted with one or more R¹⁰;
or, R⁶ and R⁷, together with the atom to which they are both bonded, optionally form a 3- to 7-membered monocyclic, bicyclic or spirocyclic cycloalkyl or heterocyclyl; wherein bicyclic or spirocyclic cycloalkyl or heterocyclyl is optionally substituted with one to three R¹⁰;
each R¹⁰ independently is H, alkyl, halogen, haloalkyl, —OH, hydroxyalkyl, -alkoxy, -alkoxyalkyl, CN or cyanoalkyl;
each of R¹¹, R¹², and R^{12′}, independently, is H, alkyl, haloalkyl, aryl, heteroaryl, cycloalkyl, or heterocyclyl, and is optionally substituted with one or more R¹⁰;
or, R¹² and R^{12′}, together with the atom to which they are both bonded, optionally form a cycloalkyl or heterocyclyl, wherein cycloalkyl or heterocyclyl is optionally substituted with one to three R¹⁰;
the heterocyclyl in each occurrence with the provision not in ring A contains one to four ring-forming heteroatoms each of which is independently oxygen (O), sulfur (S) or nitrogen (N);
the heterocyclyl in each occurrence is a monocyclic, bicyclic, bridged bicyclic, fused bicyclic, spirocyclic or polycyclic ring; and is unsaturated or partially saturated;
the heteroaryl in each occurrence contains one to four ring-forming heteroatoms each of which is independently oxygen (O), sulfur (S) or nitrogen (N);
any H is optionally replaced with deuterium (D);
p or q is independently 0, 1, 2, or 3;
or a pharmaceutically acceptable salt, tautomer, or stereoisomer thereof.

2. The compound of claim 1, wherein the compound is of Formula (II) or (III) 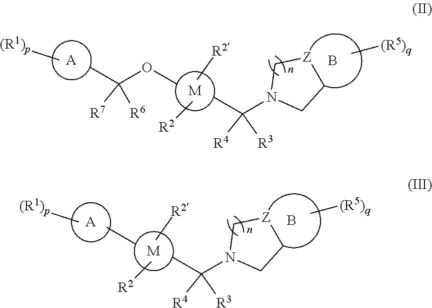
wherein
ring A is monocyclic, bicyclic or spirocyclic cycloalkyl, cycloalkenyl or heterocyclyl, aryl or heteroaryl, and is optionally substituted with one to three R¹; wherein the heteroclyl contains one or more ring-forming heteroatoms independently selected from boron (B), Silicon (Si) oxygen (O), sulfur (S) or nitrogen (N), and the ring-forming sulfur is optionally substituted with two oxo (═O) groups;
ring M is monocyclic or fused bicyclic ring, containing 0-4 ring-forming heteroatoms independently selected from N, O or S; wherein a ring-forming carbon atom is optionally substituted with oxo (═O) to form a carbonyl group;
ring B is 5 or 6 membered aryl or heteroaryl, and is optionally substituted with one to three R⁵,
or a pharmaceutically acceptable salt, tautomer, or stereoisomer thereof.

3. The compound of claim 1, wherein

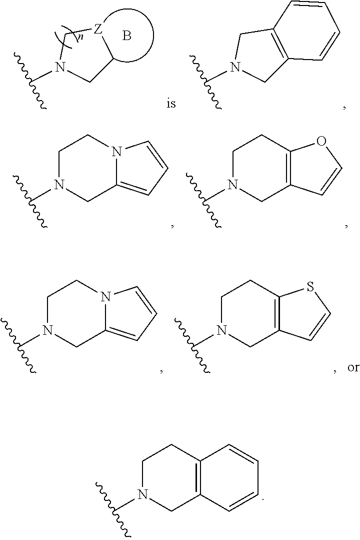

4. The compound of claim 3, wherein

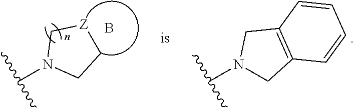

5. The compound of claim 1, wherein the compound is of Formula (IV) or (V):

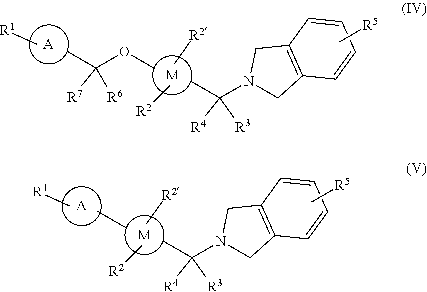

6. The compound of claim 1, wherein ring M is 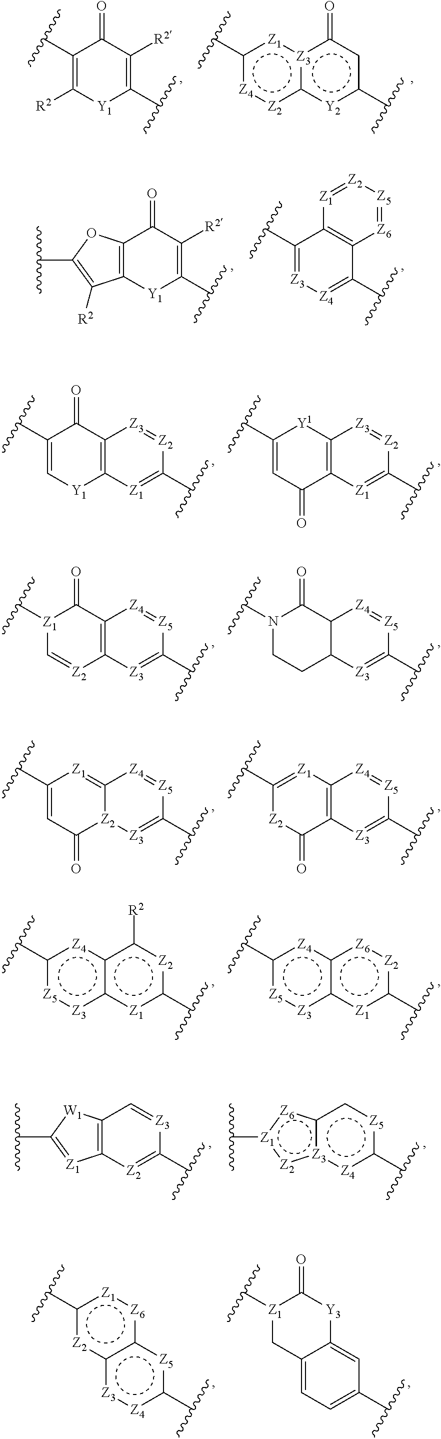 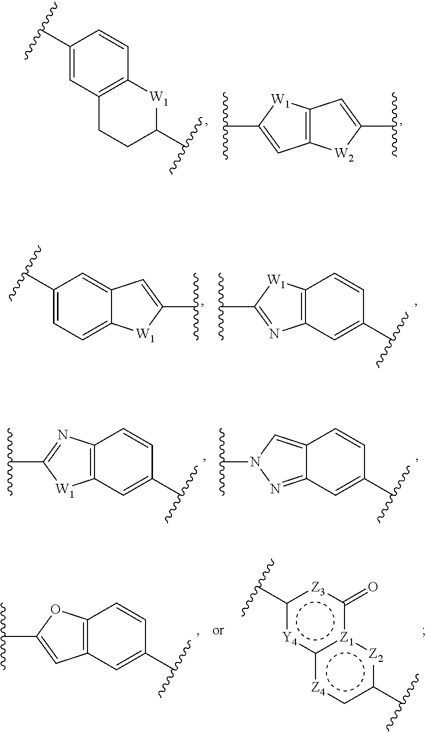
and ring M is optionally substituted with R² and R^{2′};
Y₁ is NR, O or —CRR′—;
Y₂ is N, O or ═CR—;
Y₃ is O, NR, or —CRR′—;
Y₄ is N, or CR;
each of W₁ and W₂ is independently O, S, NR, or —CRR′—;
each of Z₁, Z₂, Z₃, Z₄, Z₅ and Z₆ is independently N or CR;
R or R′ is H, alkyl, or cycloalkyl;
the dashed circle ( [symbol] ) within a ring indicates the ring contains 1, 2, or 3 double bonds as long as the valence allows,
or a pharmaceutically acceptable salt, tautomer, or stereoisomer thereof.

7. The compound of claim 6, wherein ring M is 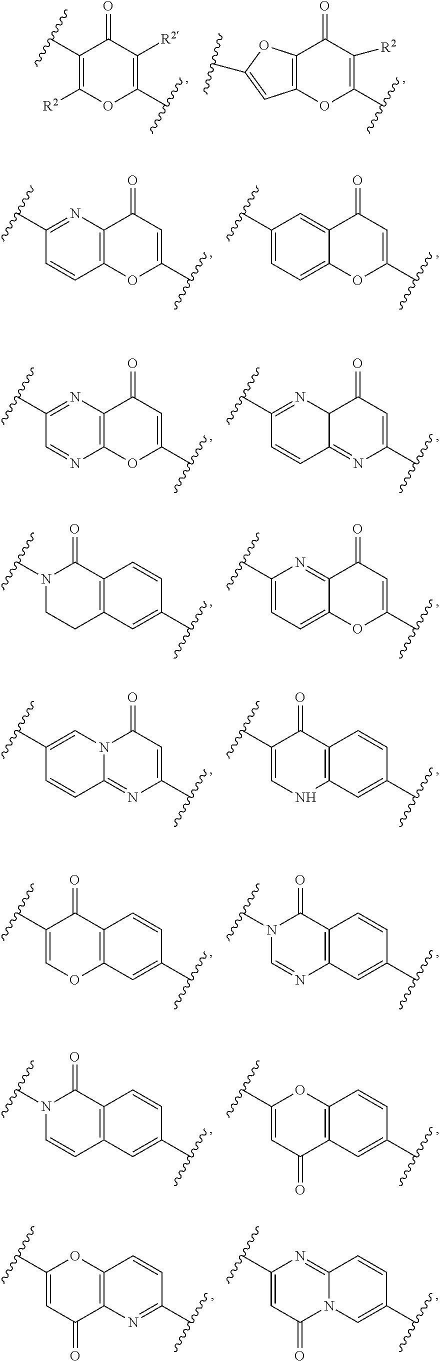 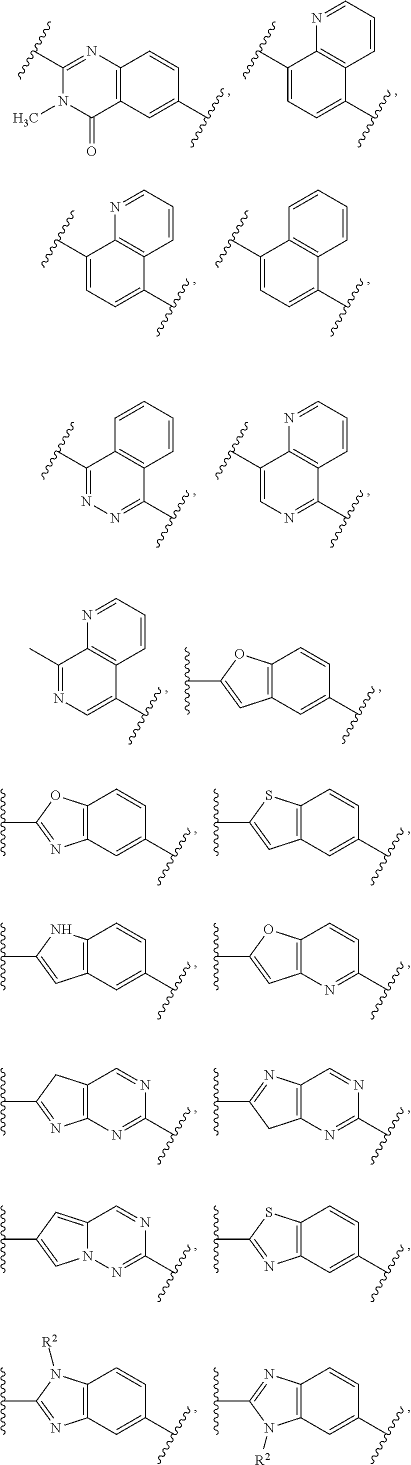 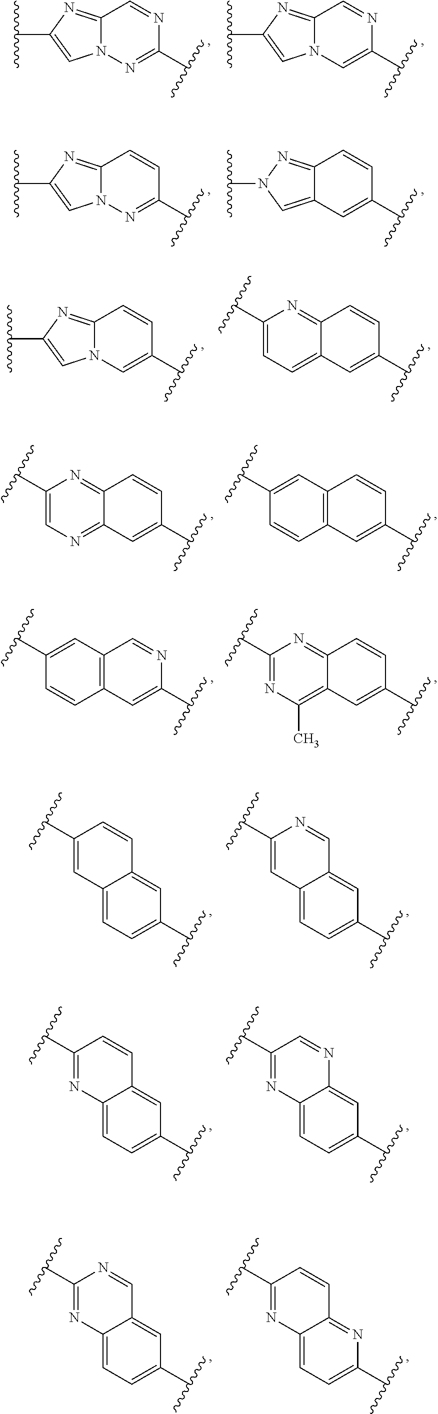 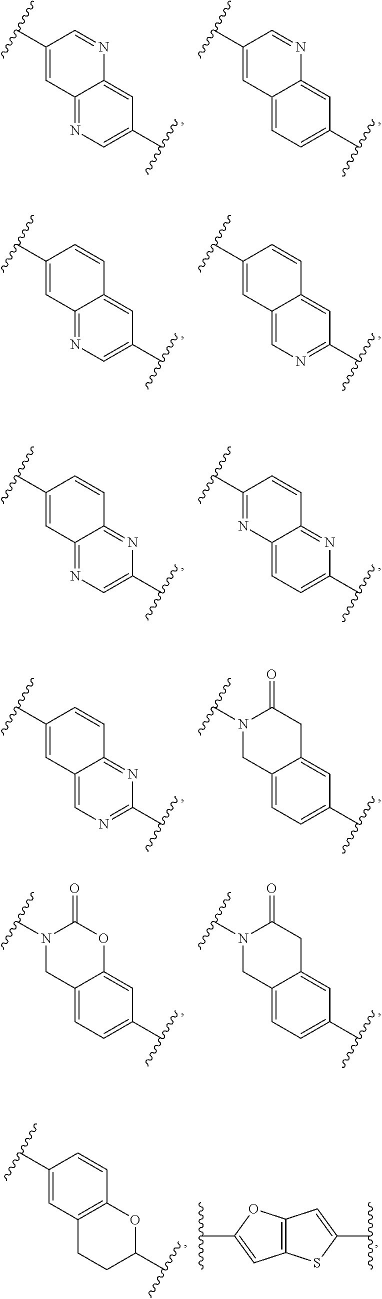 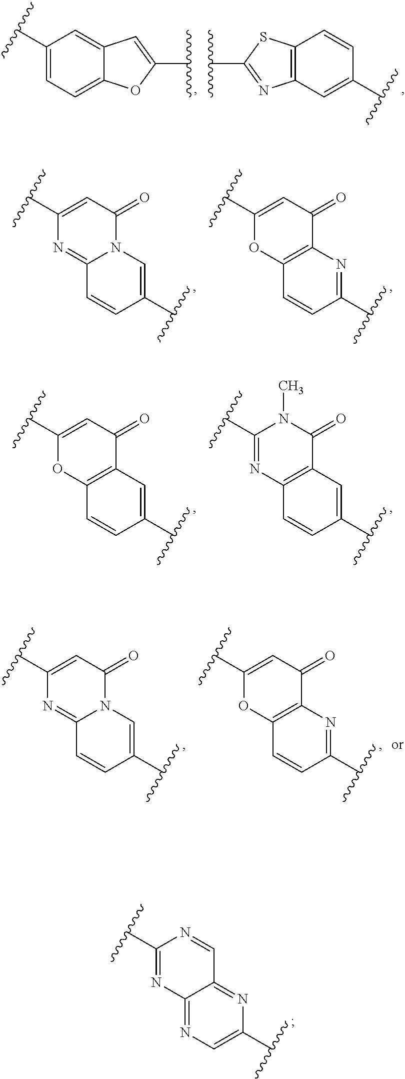
and ring M is optionally substituted with R² and R^{2′}.

8. The compound of claim 1, wherein halogen is —F or —Cl.

9. The compound of claim 1, wherein ring A is 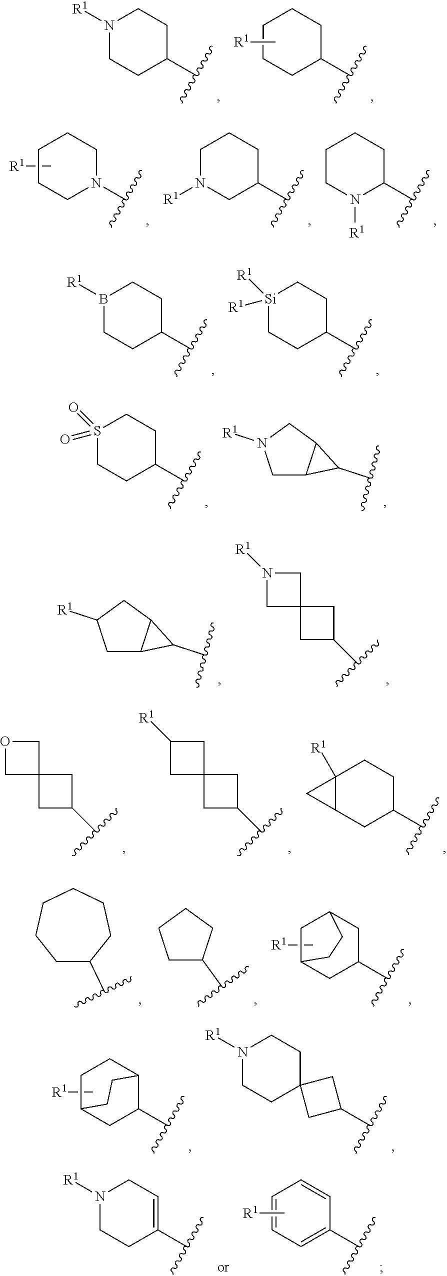
and ring A is optionally substituted with one to three R¹.

10. The compound of claim 9, wherein each R¹ is independently —OH, C₁₋₇ alkyl, —CN, —SO₂R¹¹, —COOR¹¹, —C(═O)R¹¹, —NR¹²SO₂R¹¹, —C(═O)—NR¹²R^{12′}, —SO₂NR¹²R^{12′}, —P(═O)R¹²R^{12′}, wherein each R¹¹ is independently H, cycloalkyl, heterocyclyl, haloalkyl, alkyl, aryl, or heteroaryl, wherein the aryl, heteroaryl, cycloalkyl and heterocyclyl are optionally substituted with one more alkyl, halo, or haloalkyl; R¹² and R^{12′} are each independently is H, cycloalkyl, haloalkyl, or alkyl,

11. The compound of claim 1, wherein each of R³ and R⁴, independently, is H or alkyl, and any H is optionally replaced by D; or, R³ and R⁴, together with the atom to which they are both bonded, form a 3-7 membered cycloalkyl.

12. The compound of claim 1, wherein each of R⁶ and R⁷, independently, is H or alkyl.

13. The compound of claim 1, wherein the compound is selected from a group consisting of

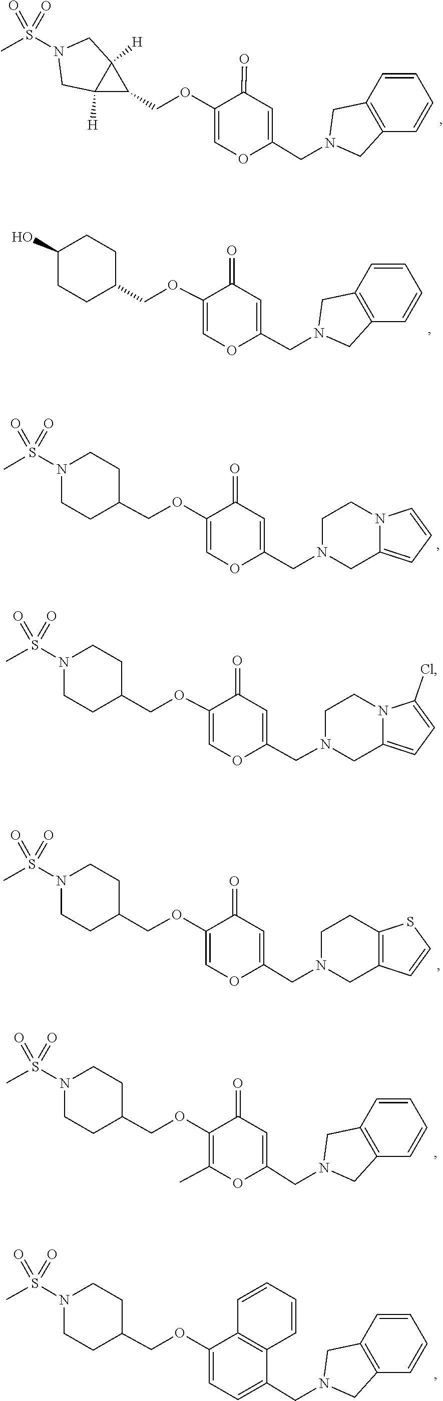

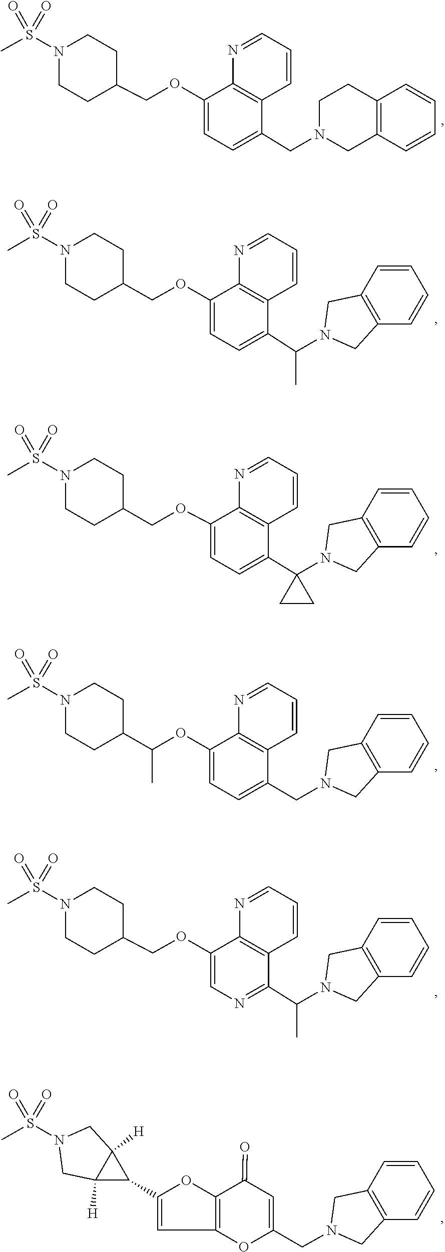 [SMILES: CS(=O)(=O)N1CCC(COc2ccc(CN3CCc4ccccc4C3)c3cccnc23)CC1 ; CC(c1ccc(OCC2CCN(S(C)(=O)=O)CC2)c2ncccc12)N1Cc2ccccc2C1 ; CS(=O)(=O)N1CCC(COc2ccc(C3(N4Cc5ccccc5C4)CC3)c3cccnc23)CC1 ; CC(Oc1ccc(CN2Cc3ccccc3C2)c2cccnc12)C1CCN(S(C)(=O)=O)CC1 ; CC(c1ncc(OCC2CCN(S(C)(=O)=O)CC2)c2ncccc12)N1Cc2ccccc2C1 ; CS(=O)(=O)N1C[C@@H]2[C@H](C1)[C@H]2c1cc2oc(CN3Cc4ccccc4C3)cc(=O)c2o1]

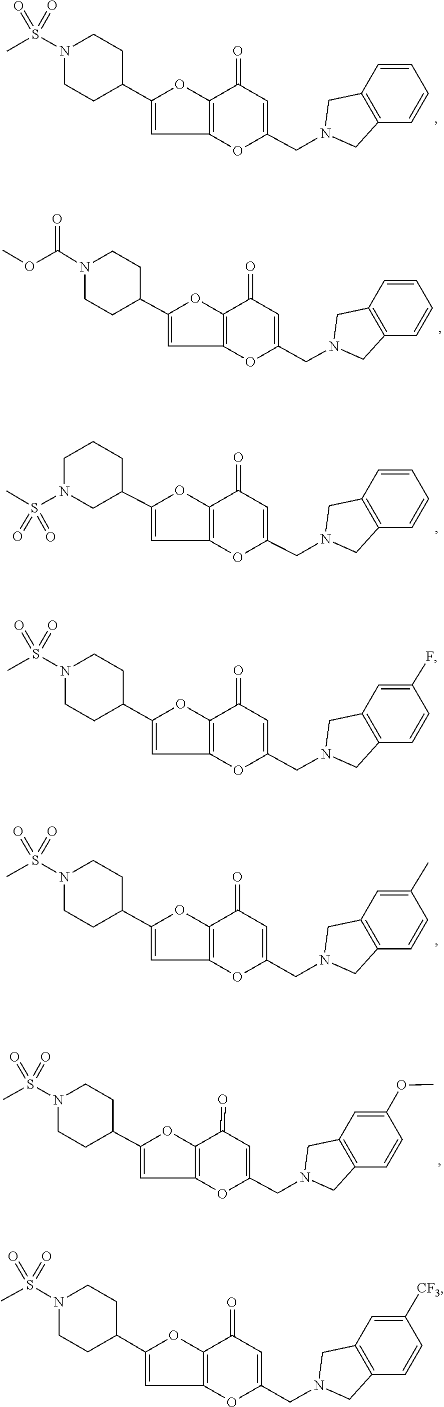

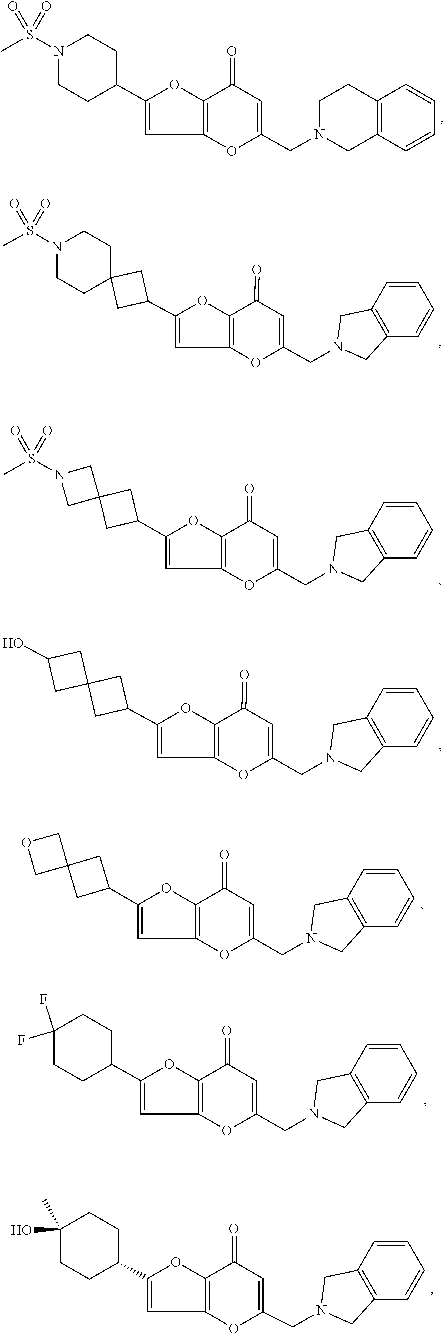

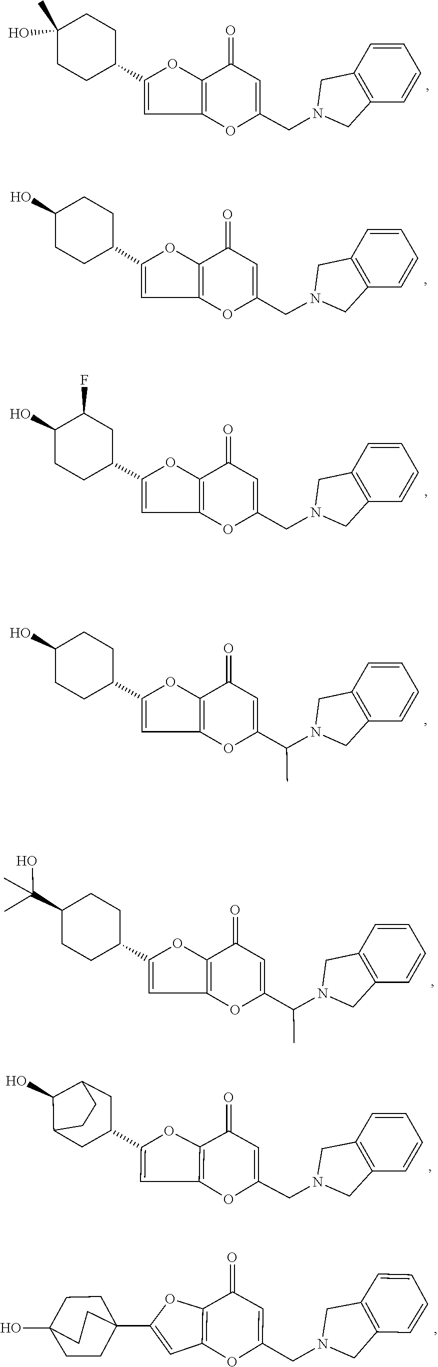

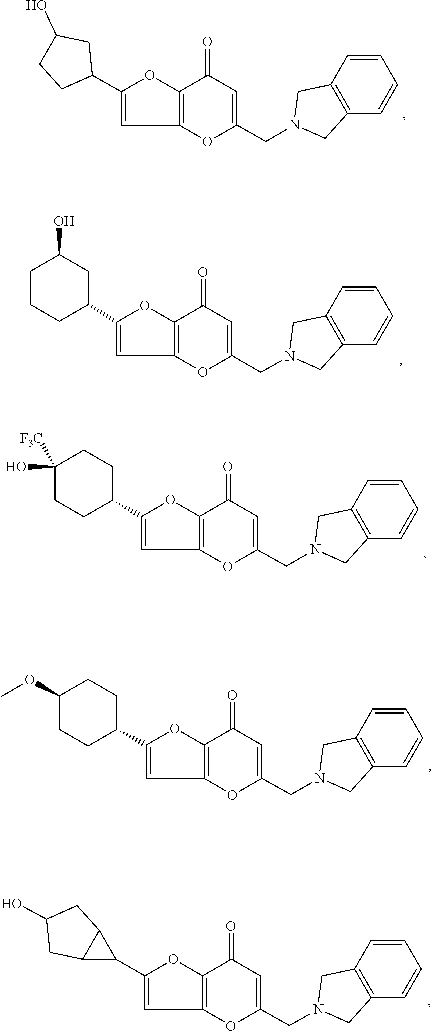 [SMILES: O=c1cc(CN2Cc3ccccc3C2)oc2cc(C3CCC(O)C3)oc12 ; O=c1cc(CN2Cc3ccccc3C2)oc2cc([C@@H]3CCC[C@@H](O)C3)oc12 ; O=c1cc(CN2Cc3ccccc3C2)oc2cc([C@H]3CC[C@@](O)(C(F)(F)F)CC3)oc12 ; CO[C@H]1CC[C@H](c2cc3oc(CN4Cc5ccccc5C4)cc(=O)c3o2)CC1 ; O=c1cc(CN2Cc3ccccc3C2)oc2cc(C3C4CC(O)CC43)oc12]

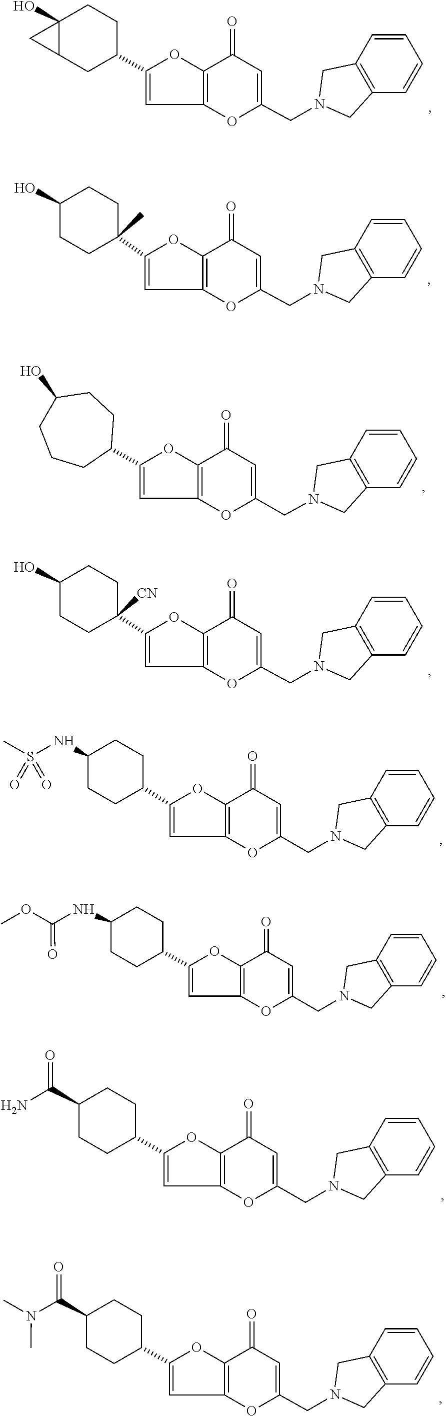

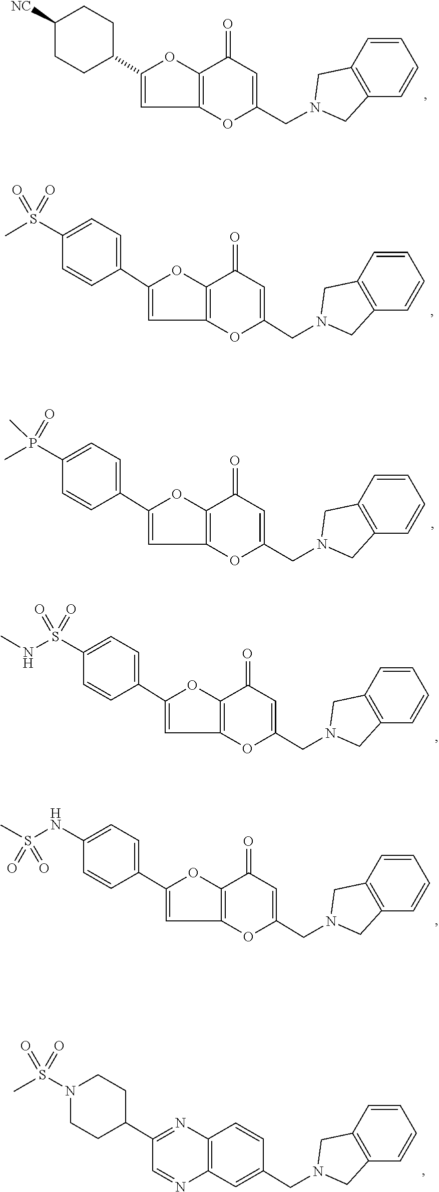 [SMILES: N#C[C@H]1CC[C@H](c2cc3oc(CN4Cc5ccccc5C4)cc(=O)c3o2)CC1 ; CS(=O)(=O)c1ccc(-c2cc3oc(CN4Cc5ccccc5C4)cc(=O)c3o2)cc1 ; CP(C)(=O)c1ccc(-c2cc3oc(CN4Cc5ccccc5C4)cc(=O)c3o2)cc1 ; CNS(=O)(=O)c1ccc(-c2cc3oc(CN4Cc5ccccc5C4)cc(=O)c3o2)cc1 ; CS(=O)(=O)Nc1ccc(-c2cc3oc(CN4Cc5ccccc5C4)cc(=O)c3o2)cc1 ; CS(=O)(=O)N1CCC(c2cnc3cc(CN4Cc5ccccc5C4)ccc3n2)CC1]

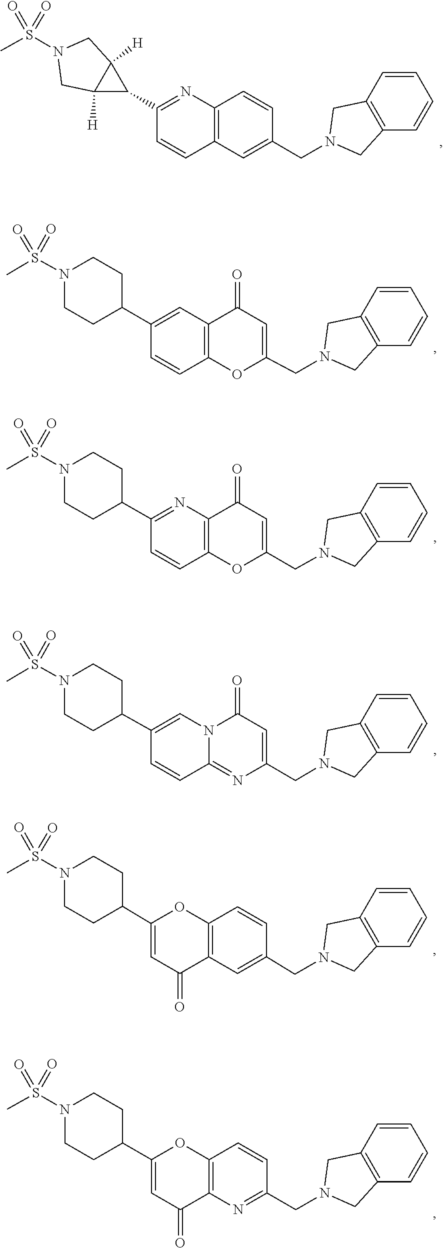 [SMILES: CS(=O)(=O)N1C[C@@H]2[C@H](C1)[C@H]2c1ccc2cc(CN3Cc4ccccc4C3)ccc2n1 ; CS(=O)(=O)N1CCC(c2ccc3oc(CN4Cc5ccccc5C4)cc(=O)c3c2)CC1 ; CS(=O)(=O)N1CCC(c2ccc3oc(CN4Cc5ccccc5C4)cc(=O)c3n2)CC1 ; CS(=O)(=O)N1CCC(c2ccc3nc(CN4Cc5ccccc5C4)cc(=O)n3c2)CC1 ; CS(=O)(=O)N1CCC(c2cc(=O)c3cc(CN4Cc5ccccc5C4)ccc3o2)CC1 ; CS(=O)(=O)N1CCC(c2cc(=O)c3nc(CN4Cc5ccccc5C4)ccc3o2)CC1]

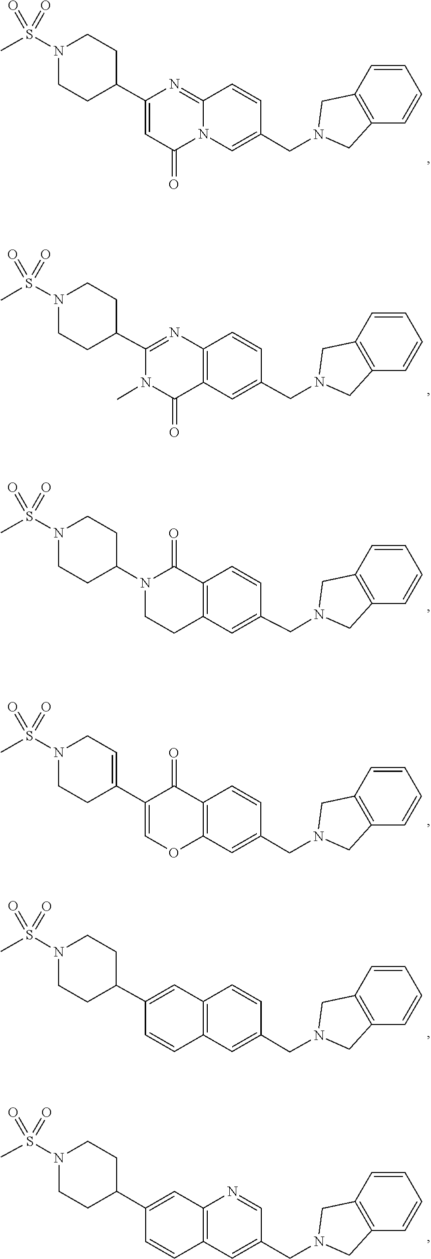 [SMILES: CS(=O)(=O)N1CCC(c2cc(=O)n3cc(CN4Cc5ccccc5C4)ccc3n2)CC1 ; Cn1c(C2CCN(S(C)(=O)=O)CC2)nc2ccc(CN3Cc4ccccc4C3)cc2c1=O ; CS(=O)(=O)N1CCC(N2CCc3cc(CN4Cc5ccccc5C4)ccc3C2=O)CC1 ; CS(=O)(=O)N1CC=C(c2coc3cc(CN4Cc5ccccc5C4)ccc3c2=O)CC1 ; CS(=O)(=O)N1CCC(c2ccc3cc(CN4Cc5ccccc5C4)ccc3c2)CC1 ; CS(=O)(=O)N1CCC(c2ccc3cc(CN4Cc5ccccc5C4)cnc3c2)CC1]

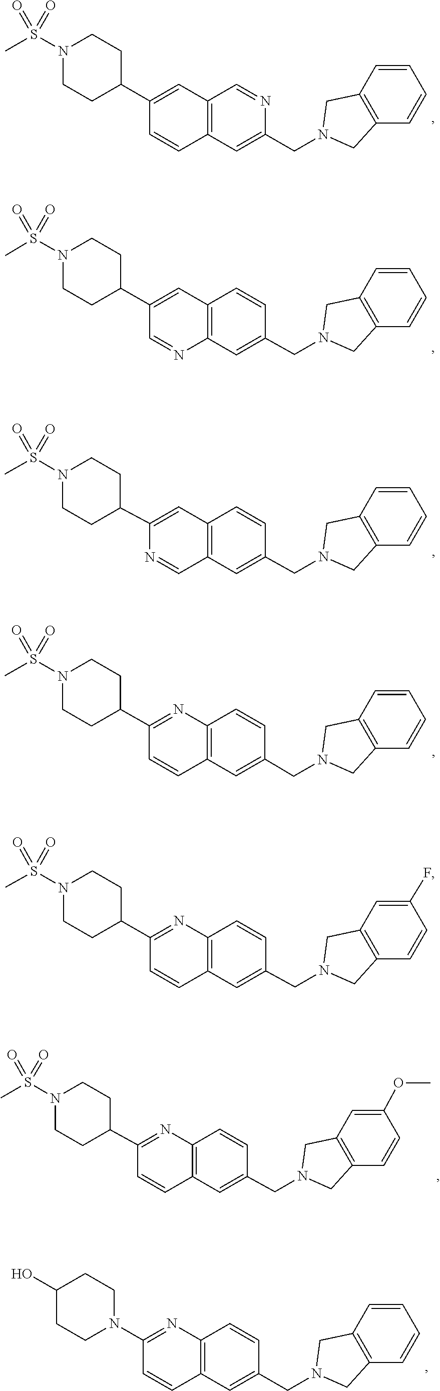

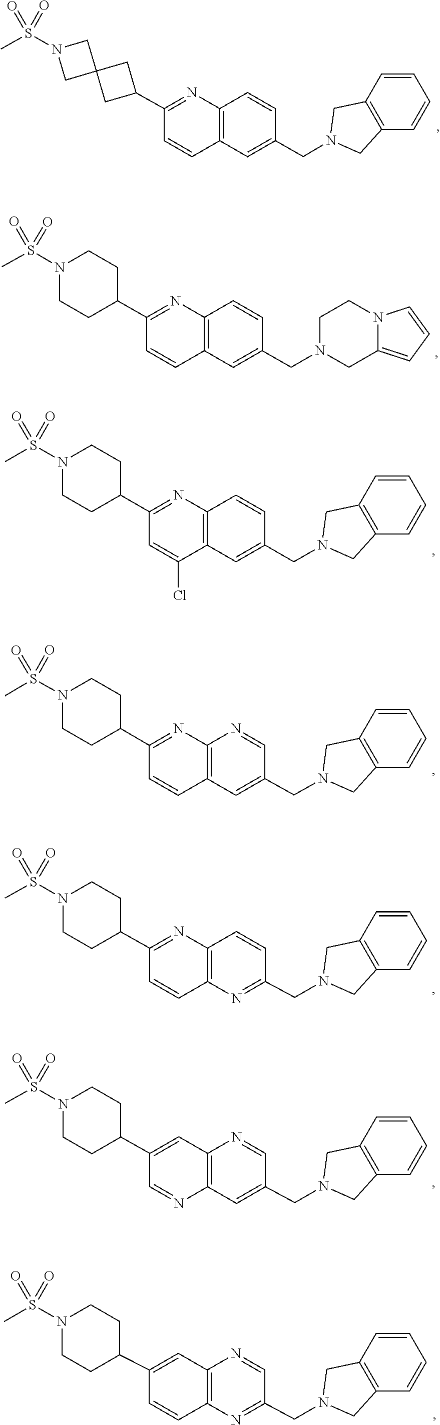

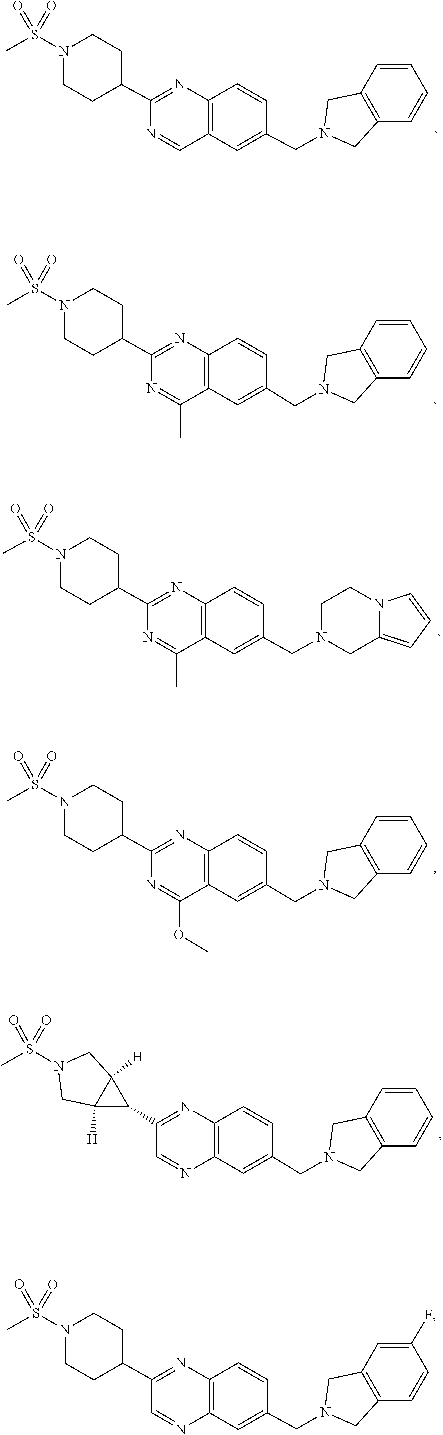 [SMILES: CS(=O)(=O)N1CCC(c2ncc3cc(CN4Cc5ccccc5C4)ccc3n2)CC1 ; Cc1nc(C2CCN(S(C)(=O)=O)CC2)nc2ccc(CN3Cc4ccccc4C3)cc12 ; Cc1nc(C2CCN(S(C)(=O)=O)CC2)nc2ccc(CN3CCn4cccc4C3)cc12 ; COc1nc(C2CCN(S(C)(=O)=O)CC2)nc2ccc(CN3Cc4ccccc4C3)cc12 ; CS(=O)(=O)N1C[C@@H]2[C@H](C1)[C@H]2c1cnc2cc(CN3Cc4ccccc4C3)ccc2n1 ; CS(=O)(=O)N1CCC(c2cnc3cc(CN4Cc5ccc(F)cc5C4)ccc3n2)CC1]

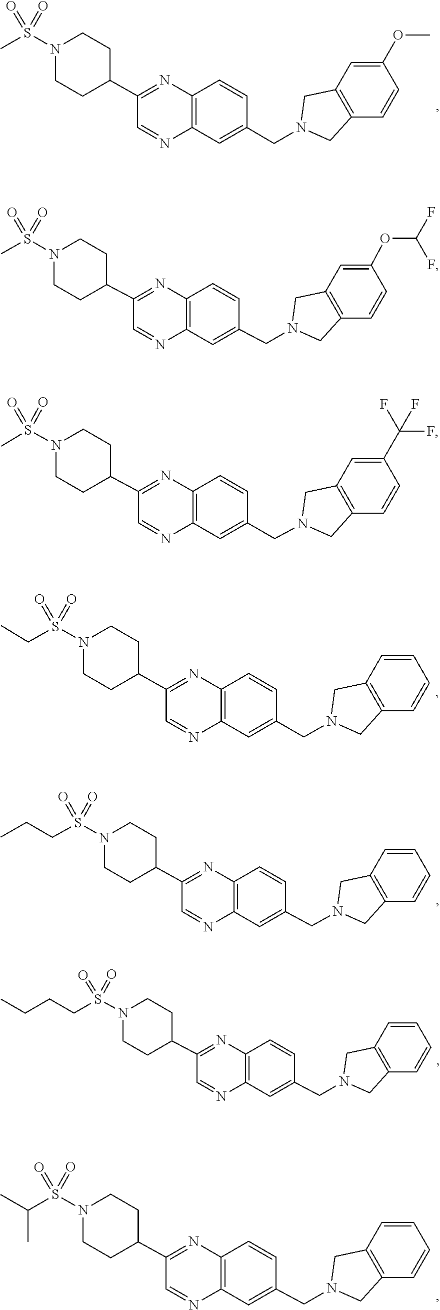

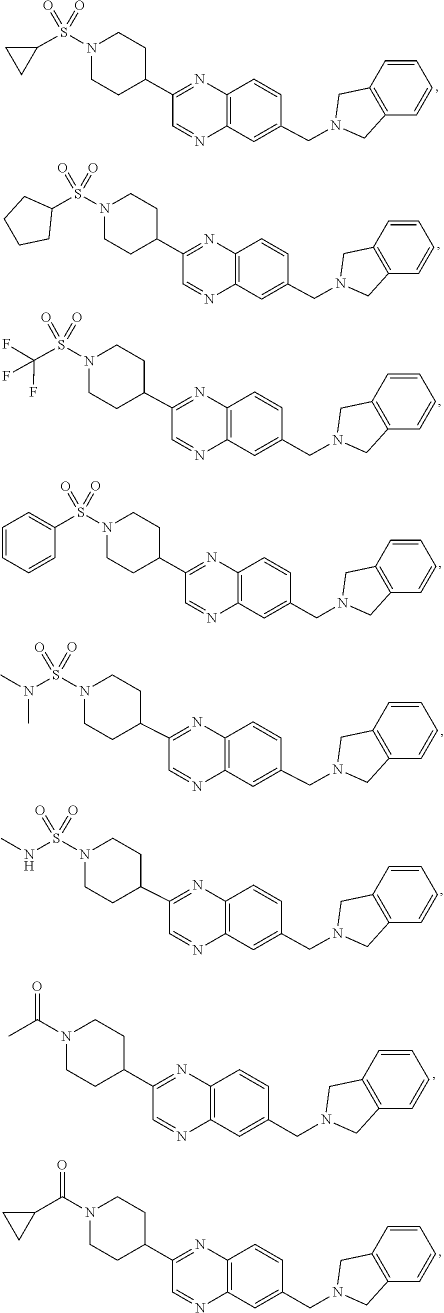

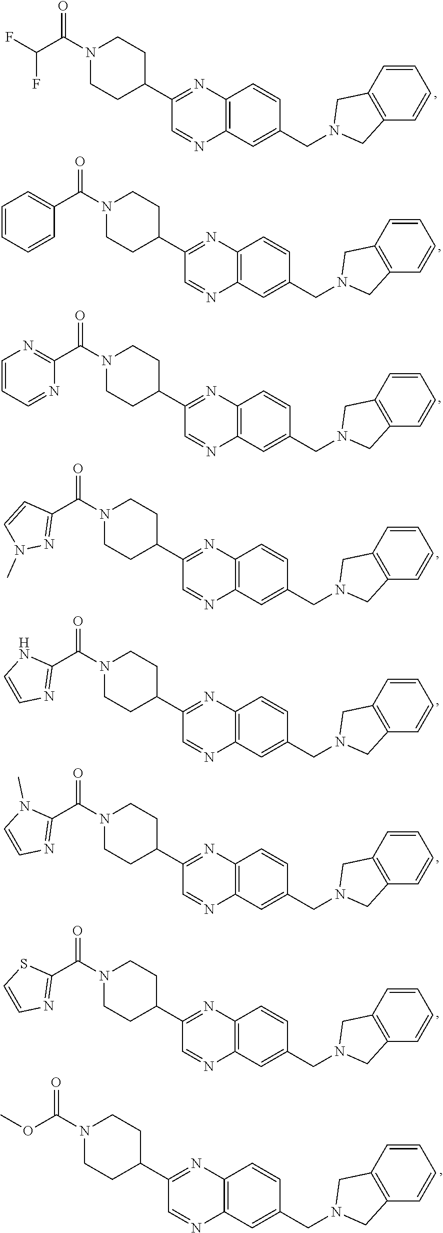

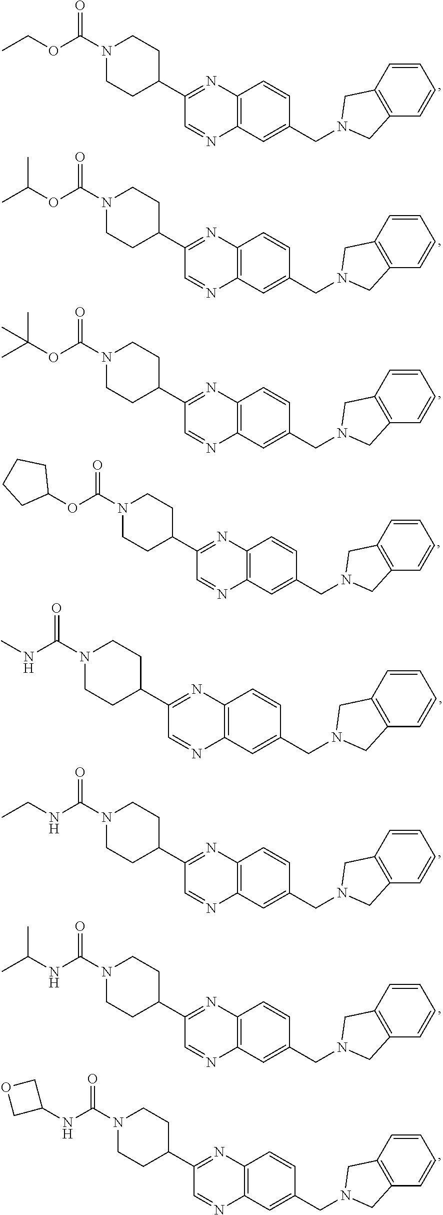

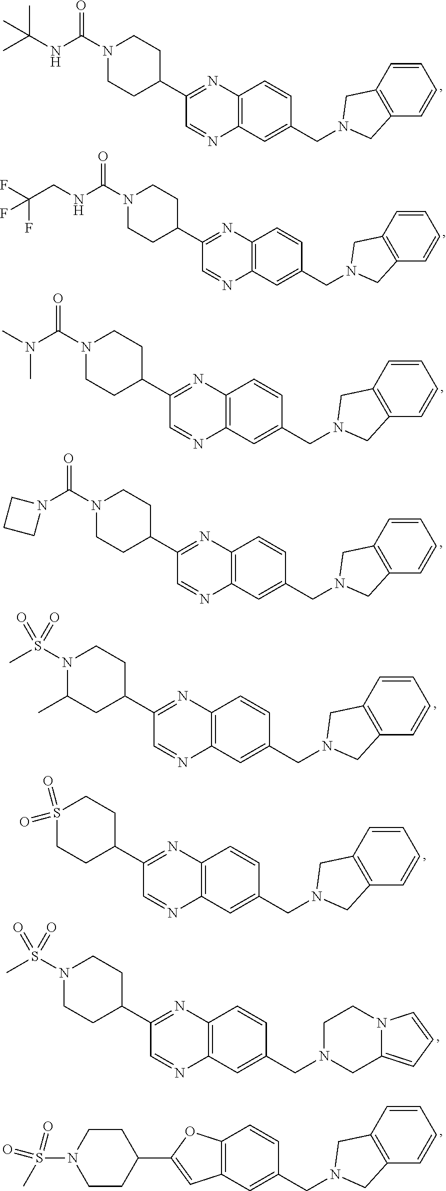

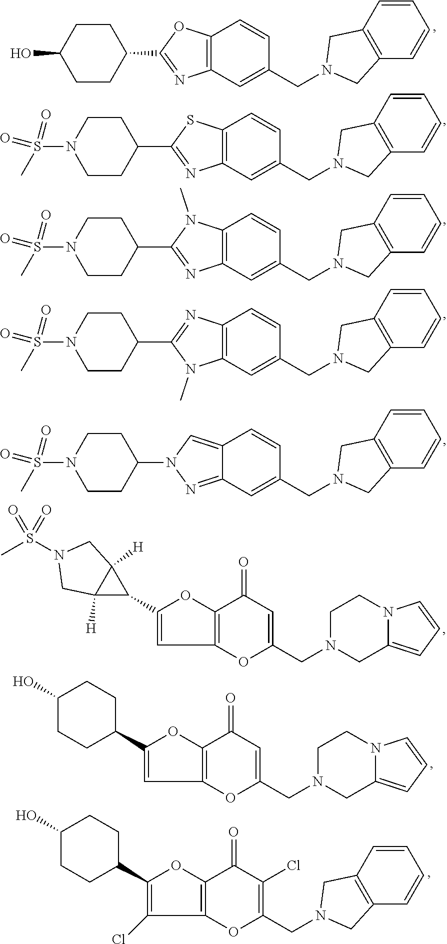

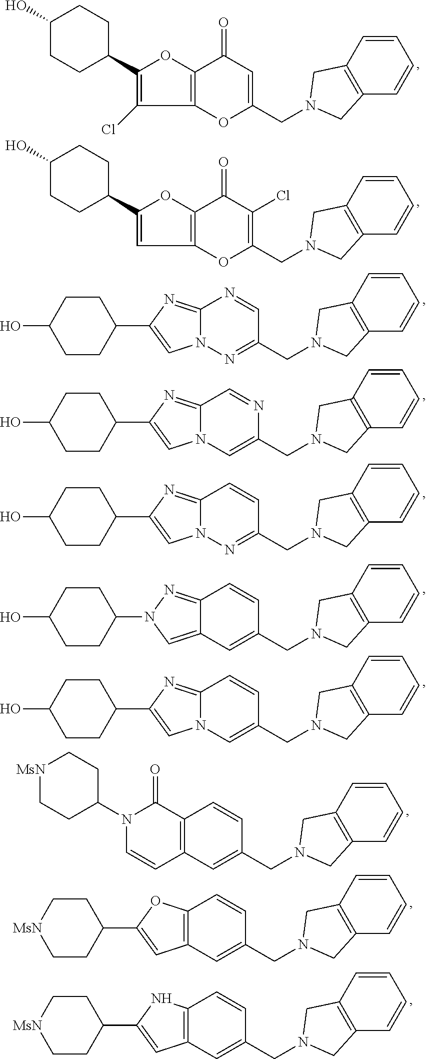

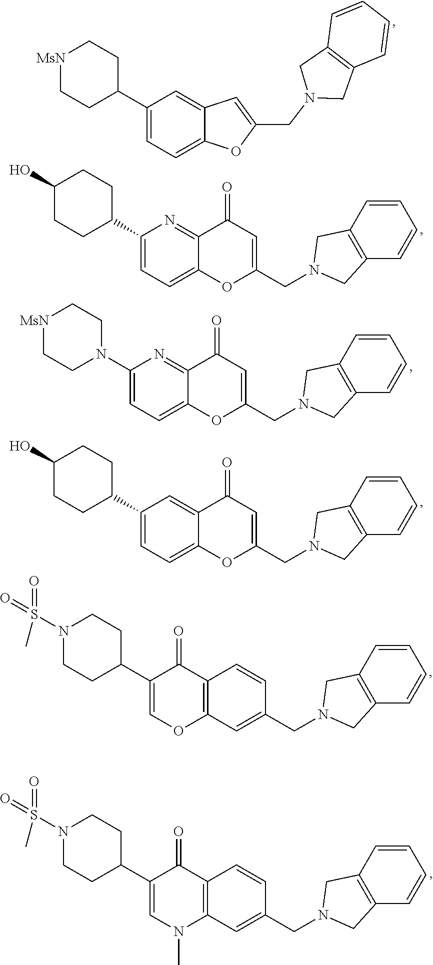

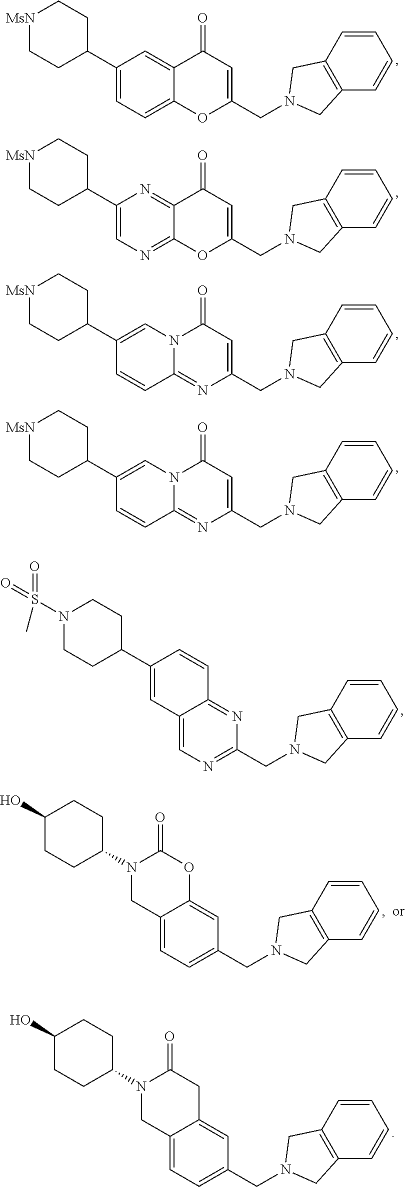

14. The compound of claim 1, wherein the compound is a cytochrome P450 11A1 (CYP11A1) inhibitor.

15. A pharmaceutical composition, comprising a therapeutically effective amount of a compound of claim 1, and a pharmaceutically acceptable carrier or excipient.

16. A method for treating a disease or condition mediated by steroid hormone receptor, comprising administration to a subject in need thereof a therapeutically effective amount of a compound of claim 1.

17. The method of claim 16, wherein the steroid hormone receptor is androgen receptor (AR).

18. The method of claim 16, wherein the condition or disease mediated by steroid hormone receptor is cancer.

19. The method of claim 18, wherein the cancer is prostate cancer or breast cancer.

20. The method of claim 19, wherein the prostate cancer is castration-resistant prostate cancer (CRPC).

21. (canceled)
